(19) United States
(12) Patent Application Publication
(10) Pub. No.: US 2026/0290579 A1
(43) Pub. Date: Sep. 24, 2026

(54) AI Therapist Assistant Using Agentic Workflow Infrastructure

(71) Applicant: Anonymous Health, Redwood City, CA (US)

(72) Inventors: Chwen-Yuen Angie Chen, Palo Alto, CA (US); Geoffrey Howard Nudd, Atherton, CA (US); Joel Aronoff, Denver, CO (US); Elayne Preheim, Sacramento, CA (US); David Frederick Cristman, Walnut Creek, CA (US); Daniel Johnston, Spokane, WA (US)

(21) Appl. No.: 19/694,768

(22) Filed: Jun. 1, 2026

Related U.S. Application Data
(63) Continuation-in-part of application No. 19/299,221, filed on Aug. 13, 2025.
(60) Provisional application No. 63/765,416, filed on Feb. 28, 2025.
(60) Provisional application No. 63/766,962, filed on Mar. 4, 2025.
(60) Provisional application No. 63/816,460, filed on Jun. 2, 2025.
(60) Provisional application No. 63/869,427, filed on Aug. 24, 2025.
(60) Provisional application No. 63/895,254, filed on Oct. 7, 2025.
(60) Provisional application No. 63/895,168, filed on Oct. 7, 2025.

(51) Int. Cl. G16H 40/67; G06Q 10/109; G06Q 20/10; G16H 15/00; G16H 20/10; G16H 20/70; G16H 80/00

(52) U.S. Cl. CPC G16H 40/67; G06Q 10/1097; G06Q 20/102; G16H 15/00; G16H 20/10; G16H 20/70; G16H 80/00

(57) ABSTRACT

An intelligent therapeutic chat interface application uses an artificial intelligence platform for coordinating medication assisted treatment using an intelligent hybrid therapeutic chat interface. An AI hybrid care platform leverages AI for proactive engagement, crisis detection, and contingency management, with a voice AI framework enabling real-time communication. Therapists intervene as needed, ensuring seamless transitions between AI and human-led sessions. The backend automates workflows, tracks user engagement, and maintains compliance, delivering a scalable and secure hybrid therapy solution. Data is recorded and routed to the appropriate licensed clinician, streamlining notetaking. The result is an AI therapist assistant that can automatically create a program for a patient, in real-time, and generate engagements based on any number of events that occur before, during, and after a session. Additionally, new program content may be generated based on an existing library of program content through a workflow builder and program builder.

### CROSS-REFERENCE TO RELATED APPLICATIONS

This application is a continuation-in-part and claims priority to U.S. application Ser. No. 19/299,221, titled “AI HYBRID CARE MODELS FOR COORDINATING MEDICATION ASSISTED TREATMENT,” filed on Aug. 13, 2025, which is hereby incorporated by reference in its entirety. This application also claims priority to U.S. Provisional Patent Application Ser. No. 63/816,460, titled “Workflow-Based Configuration of Agentic AI Sessions in Mental Health Treatment” and filed on Jun. 2, 2025, to U.S. Provisional Patent Application Ser. No. 63/869,427, titled “System and Method for Automatically Scheduling and Conducting Mental Health Exercises for a User by an AI Agent” and filed on Aug. 24, 2025, to U.S. Provisional Patent Application Ser. No. 63/895,254, titled “AI Therapist Assistant Automatic Generation of a Program” and filed on Oct. 7, 2025, and to U.S. Provisional Patent Application Ser. No. 63/895,168, titled “Workflow Builder for AI Therapist Assistant Systems and Methods” and filed on Oct. 7, 2025, all of which are hereby incorporated by reference in their entirety.

### BACKGROUND

This specification generally relates to providing patients with access to care for appropriate healthcare services using artificial intelligence (AI) hybrid care models. In particular, the specification relates to a system and method for AI hybrid care models for coordinating one or more of therapy and medication assisted treatment with the support of an AI Assistant that collaborates with the clinician to engage with the patient.

Mental health issues are now at the forefront of public concern, demanding urgent attention and action. As awareness of the importance of mental well-being grows, significant challenges persist, including limited access to care, stigma, and the ongoing impacts of social and economic factors on mental health. The COVID-19 pandemic has further exacerbated these issues, particularly among young people. The impact of social isolation has led to increasing rates of depression, anxiety, and other mental health issues. Alongside this loneliness epidemic, many individuals struggle with unwanted habits that further drain their mental health, such as excessive screen time, negative self-talk, and poor sleep hygiene. To address this crisis, a multi-faceted approach is needed to break the cycle of unhealthy coping mechanisms, including substance abuse, which can escalate into addiction and compound existing mental health challenges. Addressing these interconnected issues requires innovative solutions, including expanding digital health services, implementing solutions that are low cost or incentivize treatment, and providing support for those struggling with addiction. Typically, patients seeking mental health care are given referrals to human therapists that take time to build rapport and trust in the relationship. Often, it may take several hours over several days to coordinate care with prescribing medical practitioners. Meanwhile, the advent of chatbots has shown the possibilities of using artificial intelligence in innovative ways to engage users through various channels. There is an increasing demand for the efficient utilization of healthcare provider resources, different care coordination and delivery workflows, and health care spending costs.

This background description provided herein is for the purpose of generally presenting the context of the disclosure.

### SUMMARY

The techniques introduced herein overcome the deficiencies and limitations of the prior art, at least in part, with a system and methods for providing patients with access to care using a hybrid approach that includes human therapists or other clinicians and artificial intelligence (AI) agent chatbots. Through various systems and methods, patients may be provided therapeutic or clinical engagement, coaching, diagnosis, prescribing, and/or companionship. In some embodiments, if the person chooses the AI agent chatbot for all (“AI Session”) or part of the engagement (“AI Hybrid Care Session”), they receive an incentive from the health insurer or other party. Additionally, an AI chatbot may handle requests for medication assisted treatment and coordinate delivery of care through a therapeutic chat interface, streamlining the approval process of changes to medication and using asynchronous messaging to improve the user experience. By way of example, a health insurer or other party might pay the patient $25 per attended session if they choose the lower cost but effective AI chatbot to perform all or part of the engagement, instead of the more expensive human clinician, coach, or companion. Additionally, the AI Companion may be configured, through one or more data models, to effectively manage the interactions across what would normally be several individual clinicians of different specialties and then route the information relevant to each specialty to the appropriate licensed clinicians. So, by way of example, the first 30 minutes of a therapeutic chat interface session may include a conversation with the AI Companion, followed by 20 minutes of conversation with a human therapist. The information in the conversation with the AI Companion is routed to the licensed therapist in preparation for the one-on-one conversation. The next 10 minutes may include the AI Companion asking the user about adding medication as part of the treatment, and execute a protocol of gathering information in response to a questionnaire regarding the medication. This information may then be routed to a nurse to do asynchronous prescribing. In other embodiments, the AI Companion may “prescribe” the medication, triggering a request to the supervising clinician and, responsive to the clinician approving the medication, fulfilling the prescription request. As a result, the AI hybrid care model and techniques described herein solves one of the most recurring problems in healthcare.

In an implementation, an AI Assistant is more than a chatbot. The AI Assistant is an AI agent empowered to schedule AI engagements, be deployed before, during, and after sessions, and assist with the creation of cognitive behavioral therapy (CBT) programs based on existing content developed by mental health professionals. In this way, the AI Assistant, through the use of agentic workflow infrastructure, can do more than follow fixed steps; it can plan, choose actions, use tools, and adjust based on results with relatively little human intervention. In practice, the AI Assistant may breakdown a command from a human therapist into tasks, making the AI Assistant useful for multi-step work that needs judgment and adaptation, not just simple automation.

The features and advantages described herein are not all-inclusive and many additional features and advantages will be apparent in view of the figures and description. Moreover, it should be understood that the language used in the present disclosure has been principally selected for readability and instructional purposes, and not to limit the scope of the subject matter disclosed herein.

### BRIEF DESCRIPTION OF THE DRAWINGS

The disclosure is illustrated by way of example, and not by way of limitation in the figures of the accompanying drawings in which like reference numerals are used to refer to similar elements.

FIG. 1 is a high-level block diagram illustrating one implementation of an example system for coordinating medication assisted treatment using an intelligent hybrid therapeutic chat interface.

FIG. 2 is a block diagram illustrating one implementation of a computing device including a therapeutic chat interface application, a supervising application, and various modules for coordinating medication assisted treatment.

FIG. 3 is a high-level block diagram illustrating one implementation of an example system for coordinating medication assisted treatment by enabling clinicians and users to interact via an intelligent hybrid therapeutic chat interface.

FIG. 4A depicts graphical representations of example user interfaces of a user accessing the therapeutic chat interface to select an AI Companion or a human therapist.

FIGS. 4B-4D are graphical representations of example data structures used to provide a user accessing the therapeutic chat interface an example incentive.

FIG. 4E is an example graphical representation of an example user interface for clinicians to approve medication assisted treatment as coordinated through the intelligent hybrid therapeutic chat interface.

FIG. 5 is an example flowchart diagram of a method of generating a billing record associated with a user to based on delivering medication assisted treatment through a therapeutic chat interface for mental health-related issues.

FIGS. 6A-6C are graphical representations of example data structures used to provide a user accessing the therapeutic chat interface an example incentive.

FIG. 7 is an example flowchart diagram of a method of incentivizing a user to access the therapeutic chat interface for mental health-related issues.

FIGS. 8A-8D are example graphical representations of an example user interface for enabling clinicians and users to interact via an intelligent hybrid therapeutic chat interface.

FIG. 9 is a high-level block diagram illustrating a second implementation of an example system for coordinating medication assisted treatment using AI therapist assistant.

FIGS. 10A-10B are example flowchart diagrams of a method of operation of an AI therapist assistant automatically scheduling and conducting mental health exercises for a user in accordance with the present disclosure.

FIG. 11 is an example flowchart diagram of a method of providing workflow-based configuration of agentic AI sessions in mental health treatment.

FIG. 12 is an example flowchart diagram of a method of operation of building a program in accordance with the present disclosure.

FIGS. 13A-13B are an example flowchart diagram of a method of operation of workflow builder in accordance with the present disclosure.

FIG. 14 is a block diagram illustrating an implementation of the AI therapist assistant in more detail.

FIG. 15 is a block diagram illustrating an implementation of a workflow builder in accordance with the present disclosure.

FIG. 16 is a block diagram illustrating an implementation of a program builder in more detail.

FIG. 17 is a high-level block diagram illustrating an example module for workflow data management according to an embodiment.

FIGS. 18A-18E are graphical representations of example user interfaces of a supervising user accessing workflow-based configuration of agentic AI sessions in mental health treatment.

FIGS. 19A-19C are example graphical representations of an example user interface for assigning an AI therapist assistant to schedule and conduct mental health exercises in accordance with the present disclosure.

FIGS. 20A-20D are example graphical representations of user interfaces for creating new assignments for the AI therapist assistant to perform in accordance with the present disclosure.

FIG. 21 is an example graphical representation of an example user interface for creating a workflow using the workflow builder in accordance with the present disclosure.

### DETAILED DESCRIPTION

While the present disclosure may describe the techniques herein in the context of a user seeking mental health services via AI hybrid care sessions with a combination of human therapists, clinicians, and AI agent chatbots that provide therapeutic or clinical engagement, coaching, diagnosis, prescribing, companionship and the like, it should be understood that the architecture, principles, and components of the present disclosure may also be used to provide other kinds of interactions with humans and AI agent chatbots for various purposes. The systems and methods described below may be applied to various other medical care, coordination, and delivery procedures in addition to those specifically set forth below. A system or interface for selecting among a set of human therapists or other clinicians and artificial intelligence (AI) agent chatbots that provide therapeutic or clinical engagement, coaching, diagnosis, prescribing, and/or companionship, wherein if the person chooses the AI agent chatbot for all (“AI Session”) or part of the engagement (“AI Hybrid Care Session”), they receive an incentive from the health insurer or other party. By way of example, a health insurer or other party might pay the patient $25 per attended session if they choose the lower cost but effective AI chatbot to perform all or part of the engagement, instead of the more expensive human clinician, coach, or companion. This type of program aligns to the evidence-based for contingency management (CM), which refers to the practice of providing rewards for those struggling with addiction for achievement of goals, which may be related to the patient's treatment plan and/or engagement or use relative to an addiction or unwanted habit.

A variant may be an incentive for choosing an AI Hybrid Session wherein the AI Hybrid Session comprises some amount of time with the AI Companion exclusively, and comprises additional time with the human clinician. As a clinician prepares to take over the AI Hybrid Session, the AI Companion executes a transition to and from the clinician. In the transition from the AI Companion to the clinician part of the session, the AI Companion summarizes the discussion that had just happened with the patient and prompts the patient or clinician(s) to see if they have further comments or questions before transitioning the session to the clinician.

An AI Hybrid Session may be instantiated in several ways, including: 1) the AI Companion begins the session with the patient, then clinician joins at some time later, at which point there is a transition discussion, then the clinician leads the AI Hybrid Session thereafter; 2) the AI Hybrid Session begins with the clinician leading the start of the session, then the clinician at some point activates the AI Companion and preferably designates one or more content workflows for the Companion to execute, such as Cognitive Behavioral Therapy (CBT) exercises, outcomes data collection, screeners, clinician form completion, and the like. There is a transition between the clinician, patient and AI Companion, and then the AI Companion takes over. The clinician stays on while the AI Companion leads the discussion, and the clinician may listen, add comments, and/or take over when desired. 3) A variant of 2) above where the clinician leaves the session entirely after the 3-way transition, and the AI Companion takes over leadership of the session and concludes it when it is complete.

Another variant may be an incentive for choosing an AI Session wherein the encounter is exclusively with the AI agent chatbot, and the session is monitored real-time, near-real time, or asynchronously by a clinician or supervisor. In another variant, the AI Hybrid Session or AI Session is not monitored by a clinician or supervisor, but it is monitored by a separate AI agent that is trained to provide QA and supervision to the chat, and which alerts the clinician or supervisor based on certain criteria (e.g., if the patient expresses suicidality, inclination to violence; delusions; crisis; or any other factors). A variant of this model may apply to the treatment of other conditions such as loneliness, depression, medical conditions, mental health conditions, or any other clinical condition or diagnosis. It may also apply to engagement models oriented at preventative care (e.g. physical therapy, nutrition, sleep, or exercise coaching).

Moreover, the AI Companion could lead the patient through an asynchronous diagnosis and prescribing process. In one instantiation, the information collected by the AI Companion for prescribing may in turn be shared with a human, licensed nurse practitioner, medical doctor, or other licensed clinician. In another instantiation, a continuous appointment with more than one AI agent may be offered, each possibly having its own avatar or digital representation, wherein one AI agent represents a therapist, companion, or coach, and the other (or same) AI agent, at times, represents a prescribing medical clinician. The data from each AI chatbot agent could be routed to the appropriate licensed clinician, thus allowing the AI chatbot to function as a tool that routes information according to the respective licensures of a supervising clinician, and/or to operate under the licensure of the respective clinicians.

This approach has considerable advantages, and most notably, allows the patient to potentially receive the benefit of several appointments across clinical specialties in one continuous session as facilitated by the AI agent(s), with information and supervision routed to the appropriate clinicians and administrators across disciplines and as appropriate and required. Moreover, this improves the efficiency of processing user-generated input and improving the performance of chatbots used to interact with users in a mental health setting, thus improving the technology involved in training and deploying the chatbots. Previously, patients had to physically attend several different appointments with real humans at once to meet all their clinical needs. This coordination problem is one of the most significant and recurring problems in healthcare across many fields. In this way, one or more AI agents can deliver messages that engage with the patient with a spectrum of autonomy as permissible by law and regulation, and the information and exchange may be routed to, evaluated, and/or processed by the appropriate clinician. Additionally, because a chatbot, or an AI Companion as described herein, is accessible on demand, twenty four hours a day and seven days a week, more data may be provided by the user over time, improving the data model(s) used by the AI Companions to identify approaches that are more personalized for the user based on the improved machine learning (ML) models, in an embodiment. Because natural language understanding (NLU) models may be used by the AI agentic chatbots, a specific NLU model may be developed and improved to better understand an intent by a user, based on the amount of messages over time.

In an implementation, the AI Companion is an AI Assistant to a human therapist. A human therapist may treat the AI Assistant like a co-pilot, making complex commands and/or instructions for the AI Assistant to interpret, create a plan, and adjust as needed using an agentic workflow infrastructure. The AI Assistant, or AI Therapist Assistant, has the ability to schedule AI engagements, be deployed before, during, and/or after therapy sessions, and create programs based on existing content, such as cognitive behavioral therapy (CBT) style exercises, thought records, thought-deconstruction prompts, psychoeducation, and skill-based nudges. These AI engagements may be embedded in calls, texts, or application screens. The AI Assistant may also enable users to respond to digital homework prompts, check-ins, mood tracking, and CBT-style reflection exercises delivered via an application or therapeutic chat interface. The AI Assistant may have AI-driven risk detection and escalation to clinicians, in an implementation.

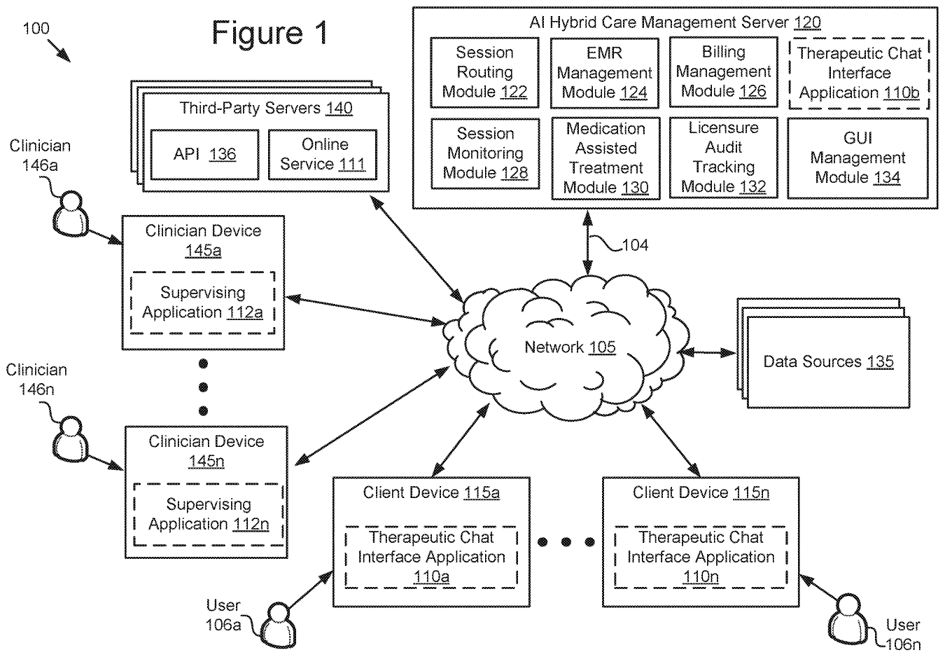

FIG. 1 is a high-level block diagram illustrating one implementation of an example system 100 for providing users with access to care using an intelligent hybrid therapeutic chat interface. The illustrated system 100 may include one or more client devices 115a . . . 115n that can be accessed by users, an AI hybrid care management server 120, a plurality of data sources 135, a plurality of third-party servers 140, and one or more clinician devices 145a . . . 145n which are communicatively coupled via a network 105 for interaction and electronic communication with one another. In FIG. 1 and the remaining figures, a letter after a reference number, e.g., “115a,” represents a reference to the element having that particular reference number. A reference number in the text without a following letter, e.g., “115,” represents a general reference to instances of the element bearing that reference number

The network 105 may be a conventional type, wired or wireless, and may have numerous different configurations including a star configuration, token ring configuration, or other configurations. Furthermore, the network 105 may include any number of networks and/or network types. For example, the network 105 may include a local area network (LAN), a wide area network (WAN) (e.g., the Internet), virtual private networks (VPNs), mobile (cellular) networks, wireless wide area network (WWANs), WiMAX® networks, Bluetooth® communication networks, peer-to-peer networks, near field networks (e.g., NFC, etc.), and/or other interconnected data paths across which multiple devices may communicate, various combinations thereof, etc. The network 105 may also be coupled to or include portions of a telecommunications network for sending data in a variety of different communication protocols. In some implementations, the network 105 may include Bluetooth communication networks or a cellular communications network for sending and receiving data including via short messaging service (SMS), multimedia messaging service (MMS), hypertext transfer protocol (HTTP), direct data connection, WAP, email, etc. In some implementations, the data transmitted by the network 105 may include packetized data (e.g., Internet Protocol (IP) data packets) that is routed to designated computing devices coupled to the network 105. Although FIG. 1 illustrates one network 105 coupled to the client devices 115, the AI hybrid care management server 120, the plurality of data sources 135, the plurality of third-party servers 140, and the clinician devices 145, in practice one or more networks 105 can be connected to these entities.

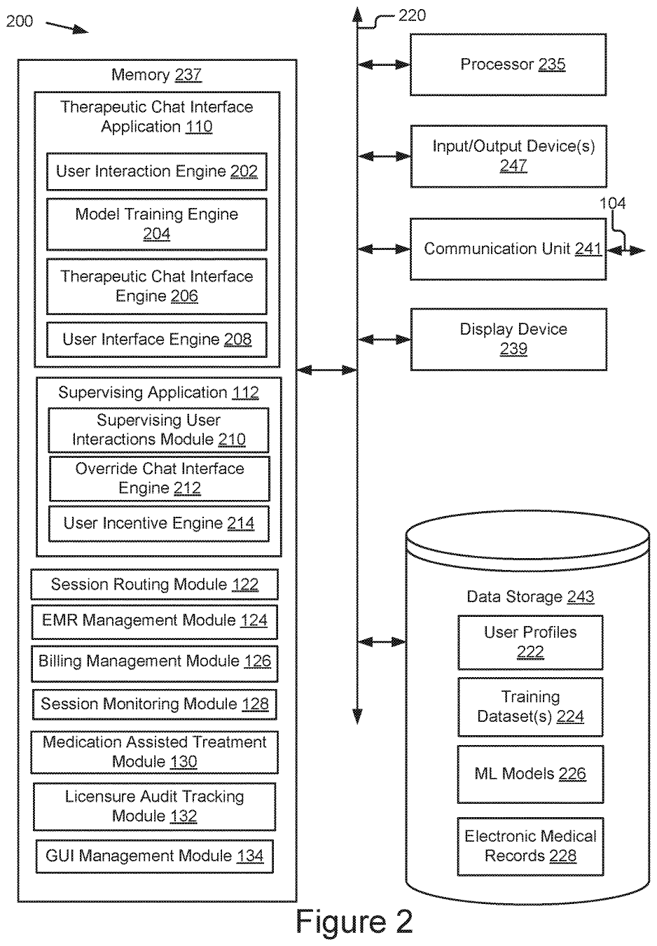

The client devices 115a . . . 115n (also referred to individually and collectively as 115) may be computing devices having data processing and communication capabilities. In some implementations, a client device 115 may include a memory, a processor (e.g., virtual, physical, etc.), a power source, a network interface, software and/or hardware components, such as a display, graphics processing unit (GPU), wireless transceivers, keyboard, camera (e.g., webcam), sensors, firmware, operating systems, web browsers, applications, drivers, and various physical connection interfaces (e.g., USB, HDMI, etc.). The client devices 115a . . . 115n may couple to and communicate with one another and the other entities of the system 100 via the network 105 using a wireless and/or wired connection. Examples of client devices 115 may include, but are not limited to, laptops, desktops, tablets, mobile phones (e.g., smartphones, feature phones, etc.), server appliances, servers, virtual machines, smart TVs, media streaming devices, user wearable computing devices (e.g., fitness trackers, etc.) or any other electronic device capable of accessing a network 105. In the example of FIG. 1, the client device 115a is configured to implement a therapeutic chat interface application 110a described in more detail below. The client device 115 includes a display for viewing information provided by one or more entities coupled to the network 105. For example, the client device 115 may be adapted to send and receive data to and from the AI hybrid care management server 120. While two or more client devices 115 are depicted in FIG. 1, the system 100 may include any number of client devices 115. In addition, the client devices 115a . . . 115n may be the same or different types of computing devices. The client devices 115a . . . 115n may be associated with the users 106a . . . 106n. For example, users 106a . . . 106n may include different types of people struggling with mental health issues, including people who want to eliminate unwanted habits, young people feeling isolated, elderly people feeling loneliness, and folks struggling with addiction and/or substance abuse. Each client device 115 may be associated with a data channel, such as a mobile application running on a user's smartphone, a personal computer accessing the therapeutic chat interface application via a web browser, a wearable device running an application to access the therapeutic chat interface, etc. These data channels may collect data related to one or more users and provide that data to the entities coupled to the network 105. In some implementations, the client devices 115 may be implemented as a computing device 200 as will be described below with reference to FIG. 2.

The clinician devices 145a . . . 145n may include, but are not limited to, laptops, desktops, tablets, mobile phones (e.g., smartphones, feature phones, etc.), server appliances, servers, virtual machines, smart TVs, media streaming devices, user wearable computing devices (e.g., fitness trackers, etc.) or any other electronic device capable of accessing a network 105. Authorized personnel who are trained to use the clinician device 145 may obtain the user's medical information. For example, the authorized personnel may include physicians, therapists and clinical staff. In some implementations, the clinician device 145 may cooperate with the client device 115 to allow authorized personnel to communicate with other entities of the system 100. For example, the client device 115 receives a report associated with a patient including an assessment result from the clinician device 145, and sends the report to the AI hybrid care management server 120 for storage and analysis. Clinician devices 145 may also run supervising applications 112 used to monitor the AI agent chatbots conversations with users.

The supervising application 112 may include software and/or logic to provide the functionality for providing clinicians with access to the AI hybrid care provided through the intelligent hybrid therapeutic chat interface on the therapeutic chat interface application 110. In some implementations, the supervising application 112 may be implemented using programmable or specialized hardware, such as a field-programmable gate array (FPGA) or an application-specific integrated circuit (ASIC). In some implementations, the therapeutic chat interface application 110 may be implemented using a combination of hardware and software. In one implementation, the supervising applications 112 are stored and executed on clinician devices 145 alone. In other implementations, the supervising application 112 and the therapeutic chat interface application 110 may be stored and executed on various combinations of the clinician devices 145, the client device 115, the data sources 135, the third-party servers 140, and the AI hybrid care management server 120, or by any one of the clinician devices 145, the client devices 115, the data sources 135, the third-party servers 140, or the AI hybrid care management server 120.

In some implementations, the supervising application 112a may be a thin-client application with some functionality executed on the clinician device 145 and additional functionality executed on the AI hybrid care management server 120 by the therapeutic chat interface application 110b. In some implementations, the supervising application 112 may generate and present various user interfaces to perform these acts and/or functionality, which may in some cases be based at least in part on information received from the AI hybrid care management server 120, the client device 115, the clinician device 145, one or more of the third-party servers 140 and/or the data sources 135 via the network 105. In some implementations, the supervising application 112 is code operable in a web browser, a web application accessible via a web browser, a native application (e.g., mobile application, installed application, etc.) on the clinician device 145, a combination thereof, etc. Additional structure, acts, and/or functionality of the supervising application 112 is further discussed below with reference to at least FIG. 2.

In some implementations, the supervising application 112 may require clinical users to be registered with the AI hybrid care management server 120 to access the acts and/or functionality described herein. For example, to access various acts and/or functionality provided by the supervising application 112, the supervising application 112 may require a user to authenticate their identity. For example, the supervising application 112 may require a user seeking access to authenticate their identity by inputting credentials in an associated user interface. In another example, the supervising application 112 may interact with a federated identity server (not shown) to register and/or authenticate the user by scanning and verifying biometrics including username and password, facial attributes, fingerprint, and voice.

In the example of FIG. 1, the AI hybrid care management server 120, the plurality of data sources 135, and the plurality of the third-party servers 140 may be, or may be implemented by, a computing device including a processor, a memory, applications, a database, and network communication capabilities similar to that described below with reference to FIG. 2.

In the example of FIG. 1, the AI hybrid care management server 120 may be configured to implement a therapeutic chat interface application 110b as well as a session routing module 122, an electronic medical record (EMR) management module 124, a billing management module 126, a session monitoring module 128, a medication assisted treatment module 130, a licensure audit tracking module 132, and a graphical user interface (GUI) management module 134. In some implementations, the AI hybrid care management server 120 may be a hardware server, a software server, or a combination of software and hardware. For example, the AI hybrid care management server 120 may include one or more hardware servers, virtual servers, server arrays, storage devices and/or systems, etc., and/or may be centralized or distributed/cloud-based. In some implementations, the AI hybrid care management server 120 may include one or more virtual servers, which operate in a host server environment and access the physical hardware of the host server including, for example, a processor, a memory, applications, a database, storage, network interfaces, etc., via an abstraction layer (e.g., a virtual machine manager). In some implementations, the AI hybrid care management server 120 may be a Hypertext Transfer Protocol (HTTP) server, a Representational State Transfer (REST) service, or other server type, having structure and/or functionality for processing and satisfying content requests and/or receiving content from one or more of the client devices 115, one or more of the clinician devices 145, the plurality of data sources 135, and the plurality of third-party servers 140 that are coupled to the network 105.

Also, instead of or in addition, the AI hybrid care management server 120 may implement its own application programming interface (API) for the transmission of instructions, data, results, and other information between the server 120 and other entities communicatively coupled to the network 105. For example, the API may be a software interface exposed over the HTTP protocol by the AI hybrid care management server 120. The API exposes internal data and functionality of the service hosted by the AI hybrid care management server 120 to API requests originating from one or more of the therapeutic chat interface applications 110, the plurality of data sources 135, and the plurality of third-party servers 140. In one example, the therapeutic chat interface application 110b implemented by the AI hybrid care management server 120 passes an authenticated request including a set of parameters for information to one or more of the third-party servers 140 and the data source 135 and receives an object (e.g., XML or JSON) with associated results. In some implementations, the AI hybrid care management server 120 may also include a database coupled to it (e.g., over the network 105) to store structured data in a relational database and a file system (e.g., HDFS, NFS, etc.) for unstructured or semi-structured data. In some implementations, the AI hybrid care management server 120 may include an instance of a data store that stores various types of data for access and/or retrieval by the therapeutic chat interface application 110. For example, the data store may store machine learning models for natural language understanding of user intents. Other types of user data are also possible and contemplated.

In some implementations, the AI hybrid care management server 120 sends and receives data to and from other entities of the system 100 via the network 105. For example, the AI hybrid care management server 120 sends and receives data including instructions to and from the client device 115. In some implementations, the AI hybrid care management server 120 may serve as a middle layer and permit interactions between the client device 115 and the plurality of the third-party servers 140 and the data sources 135 to flow through and from the AI hybrid care management server 120 for security and convenience. The AI hybrid care management server 120 may send data to and receive data from the other entities of the system 100 via the network 105. It should be understood that the AI hybrid care management server 120 is not limited to providing the above-noted acts and/or functionality and may include other network-accessible services. In addition, while a single AI hybrid care management server 120 is depicted in FIG. 1, it should be understood that there may be any number of AI hybrid care management servers 120 or a server cluster.

Each of the one or more third-party servers 140 may be, or may be implemented by, a computing device including a processor, a memory, applications, a database, and network communication capabilities. A third-party server 140 may be a Hypertext Transfer Protocol (HTTP) server, a Representational State Transfer (REST) service, or other server type, having structure and/or functionality for processing and satisfying content requests and/or requesting and receiving content from one or more of the client devices 115, the clinician devices 145, the data sources 135, and the AI hybrid care management server 120 that are coupled to the network 105. In some implementations, the third-party server 140 may include an online service 111 dedicated to providing access to various services and information resources hosted by the third-party server 140 via web, mobile, enterprise, and/or cloud applications. The online service 111 may obtain and store user data, user-generated data, content items (e.g., videos, text, images, etc.), and interaction data reflecting the interaction of users with the content items. In some implementations, the third-party server 140 may provide an API 136 to facilitate access of the third-party server 140 by one or more of the client devices 115, the clinician devices 145, the data sources 135, and the AI hybrid care management server 120 that are coupled to the network 105. User-generated data, as described herein, may include one or more of user profile information (e.g., user id, user preferences, user history, social network connections, primary care physicians, etc.), logged information (e.g., heart rate, activity metrics, sleep quality data, calories and nutrient data, user device specific information, historical actions, medication history, etc.), and other user specific information. In some implementations, the online service 111 allows users to share content with other users (e.g., friends, contacts, public, similar users, therapists, clinical staff, administrative staff, etc.), purchase and/or view items (e.g., e-books, videos, music, games, subscription, fitness products, prescription refill, laboratory results, assessment results, etc.), and other similar actions. For example, the online service 111 may provide various services such as digital fitness content; personal training; running and cycling tracking service; music streaming service; mobile health (mHealth) service; video streaming service; web mapping service; multimedia messaging service; electronic mail service; a calendar service; news service; news aggregator service; social networking service; location-based service; photo and video-sharing social networking service; sleep-tracking service; diet-tracking and calorie counting service; ridesharing service; online banking service; online information database service; travel service; online e-commerce marketplace; ratings and review service; restaurant-reservation service; food delivery service; search service; health and fitness service; home automation and security service; Internet of Things (IOT), multimedia hosting, distribution, and sharing service; cloud-based data storage and sharing service; a scheduling service; an enterprise clinical workflow service; a combination of one or more of the foregoing services; or any other service where users retrieve, collaborate, and/or share information, etc. It should be noted that the list of items provided above as examples for the online service 111 above are not exhaustive and that others are contemplated in the techniques described herein.

Each of the plurality of data sources 135 may be, or may be implemented by, a computing device including a processor, a memory, applications, a database, and network communication capabilities. In some implementations, the data sources may be a data warehouse, a system of record (SOR), or belonging to a data repository owned by an organization that provides real-time or close to real-time data automatically or responsive to being polled or queried by the AI hybrid care management server 120. Each of the plurality of data sources 135 may be associated with a first-party entity (e.g., server 120) or third-party entity (e.g., server 140 associated with a separate company or service provider), such as a health insurance organization, a health care organization, world health organization, an independent healthcare provider, a healthcare-related call center or customer service company, a healthcare software company, an Electronic Medical Record (EMR) software company, an Electronic Health Record (EHR) software company, a pharmacy management system, a drug research institute, a patient management software system, a clinical decision support system, a clinical workflow management system, a scheduling system, a patient-satisfaction measurement firm, a medication adherence tracking system, a public-records database, a data mining platform, a Software as a Service (SaaS) data analytics company, a data science and machine learning platform, news site, support groups, health blogs, etc. Examples of data provided by the plurality of data sources 135 may include, but is not limited to, pharmacy data, physician-patient encounter data, clinical data, patient data, EMR, EHR, patient diagnosis data, patient procedures, appointment notes, socioeconomic data, social determinant data, demographic data, health plan data, prescription data, call center data, appointment schedule data, disposition data, calendar data, medication data, pharmaceutical data, survey data, medication adherence data, machine learning models, machine learning-based data analysis results, etc. In some implementations, each of the plurality of data sources 135 may be configured to provide or facilitate an API (not shown) that allows the therapeutic chat interface application 110 to access data and information for performing the functionality described herein.

The therapeutic chat interface application 110 may include software and/or logic to provide the functionality for providing patients with access to AI hybrid care using an intelligent hybrid therapeutic chat interface. In some implementations, the therapeutic chat interface application 110 may be implemented using programmable or specialized hardware, such as a field-programmable gate array (FPGA) or an application-specific integrated circuit (ASIC). In some implementations, the therapeutic chat interface application 110 may be implemented using a combination of hardware and software. In one implementation, the therapeutic chat interface application 110b is stored and executed on AI hybrid care management server 120 alone. In another implementation, the therapeutic chat interface application 110a, 110n is stored and executed on client device 115 alone. In other implementations, the therapeutic chat interface application 110 may be stored and executed on various combinations of the clinician devices 145, the client device 115, the data sources 135, the third-party servers 140, and the AI hybrid care management server 120, or by any one of the clinician devices 145, the client devices 115, the data sources 135, the third-party servers 140, or the AI hybrid care management server 120.

In some implementations, the therapeutic chat interface application 110a may be a thin-client application with some functionality executed on the client device 115 and additional functionality executed on the AI hybrid care management server 120 by the therapeutic chat interface application 110b. In some implementations, the therapeutic chat interface application 110 may generate and present various user interfaces to perform these acts and/or functionality, which may in some cases be based at least in part on information received from the AI hybrid care management server 120, the client device 115, the clinician device 145, one or more of the third-party servers 140 and/or the data sources 135 via the network 105. In some implementations, the therapeutic chat interface application 110 is code operable in a web browser, a web application accessible via a web browser, a native application (e.g., mobile application, installed application, etc.) on the client device 115, a combination thereof, etc. Additional structure, acts, and/or functionality of the therapeutic chat interface application 110 is further discussed below with reference to at least FIG. 2.

In some implementations, the therapeutic chat interface application 110 may require users to be registered with the AI hybrid care management server 120 to access the acts and/or functionality described herein. For example, to access various acts and/or functionality provided by the therapeutic chat interface application 110, the therapeutic chat interface application 110 may require a user to authenticate their identity. For example, the therapeutic chat interface application 110 may require a user seeking access to authenticate their identity by inputting credentials in an associated user interface. In another example, the therapeutic chat interface application 110 may interact with a federated identity server (not shown) to register and/or authenticate the user by scanning and verifying biometrics including username and password, facial attributes, fingerprint, and voice.

Other variations and/or combinations are also possible and contemplated. It should be understood that the system 100 illustrated in FIG. 1 is representative of an example system and that a variety of different system environments and configurations are contemplated and are within the scope of the present disclosure. For example, various acts and/or functionality may be moved from a server 120 to a client device 115, or vice versa, data may be consolidated into a single data store or further segmented into additional data stores, and some implementations may include additional or fewer computing devices, services, and/or networks, and may implement various functionality client or server-side. Furthermore, various entities of the system may be integrated into a single computing device or system or divided into additional computing devices or systems, etc.

FIG. 2 is a block diagram illustrating one implementation of a computing device 200 including a therapeutic chat interface application 110. The computing device 200 may also include a processor 235, a memory 237, a display device 239, a communication unit 241, an input/output device(s) 247, and a data storage 243, according to some examples. The components of the computing device 200 are communicatively coupled by a bus 220. In some implementations, the computing device 200 may be representative of the client device 115, the clinician device 145, the AI hybrid care management server 120, or a combination of the client device 115, the clinician device 145, and the AI hybrid care management server 120. In such implementations where the computing device 200 is the client device 115, the clinician device 145, or the AI hybrid care management server 120, it should be understood that the client device 115, the clinician device 145, and the AI hybrid care management server 120 may take other forms and include additional or fewer components without departing from the scope of the present disclosure. For example, while not shown, the computing device 200 may include sensors, capture devices, additional processors, and other physical configurations. Additionally, it should be understood that the computer architecture depicted in FIG. 2 could be applied to other entities of the system 100 with various modifications, including, for example, the servers 140 and data sources 135.

The processor 235 may execute software instructions by performing various input/output, logical, and/or mathematical operations. The processor 235 may have various computing architectures to process data signals including, for example, a complex instruction set computer (CISC) architecture, a reduced instruction set computer (RISC) architecture, and/or an architecture implementing a combination of instruction sets. The processor 235 may be physical and/or virtual, and may include a single processing unit or a plurality of processing units and/or cores. In some implementations, the processor 235 may be capable of generating and providing electronic display signals to a display device 239, supporting the display of images, capturing and transmitting images, and performing complex tasks including various types of feature extraction and sampling. In some implementations, the processor 235 may be coupled to the memory 237 via the bus 220 to access data and instructions therefrom and store data therein. The bus 220 may couple the processor 235 to the other components of the computing device 200 including, for example, the memory 237, the communication unit 241, the display device 239, the input/output device(s) 247, and the data storage 243.

The memory 237 may store and provide access to data for the other components of the computing device 200. The memory 237 may be included in a single computing device or distributed among a plurality of computing devices as discussed elsewhere herein. In some implementations, the memory 237 may store instructions and/or data that may be executed by the processor 235. The instructions and/or data may include code for performing the techniques described herein. For example, as depicted in FIG. 2, the memory 237 may store the therapeutic chat interface application 110. The memory 237 is also capable of storing other instructions and data, including, for example, an operating system, hardware drivers, other software applications, databases, etc. For example, the memory 237 may store a session routing module 122, an electronic medical record (EMR) management module 124, a billing management module 126, a session monitoring module 128, a medication assisted treatment module 130, a licensure audit tracking module 132, and a graphical user interface (GUI) management module 134. The memory 237 may be coupled to the bus 220 for communication with the processor 235 and the other components of the computing device 200.

The memory 237 may include one or more non-transitory computer-usable (e.g., readable, writeable) device, a static random access memory (SRAM) device, a dynamic random access memory (DRAM) device, an embedded memory device, a discrete memory device (e.g., a PROM, FPROM, ROM), a hard disk drive, an optical disk drive (CD, DVD, Blu-ray™, etc.) mediums, which can be any tangible apparatus or device that can contain, store, communicate, or transport instructions, data, computer programs, software, code, routines, etc., for processing by or in connection with the processor 235. In some implementations, the memory 237 may include one or more of volatile memory and non-volatile memory. It should be understood that the memory 237 may be a single device or may include multiple types of devices and configurations.

The bus 220 may represent one or more buses including an industry standard architecture (ISA) bus, a peripheral component interconnect (PCI) bus, a universal serial bus (USB), or some other bus providing similar functionality. The bus 220 may include a communication bus for transferring data between components of the computing device 200 or between computing device 200 and other components of the system 100 via the network 105 or portions thereof, a processor mesh, a combination thereof, etc. In some implementations, the therapeutic chat interface application 110 and various other software operating on the computing device 200 (e.g., an operating system, device drivers, etc.) may cooperate and communicate via a software communication mechanism implemented in association with the bus 220. The software communication mechanism may include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication may be configured to be secure (e.g., SSH, HTTPS, etc.).

The display device 239 may be any conventional display device, monitor or screen, including but not limited to, a liquid crystal display (LCD), light emitting diode (LED), organic light-emitting diode (OLED) display or any other similarly equipped display device, screen or monitor. The display device 239 represents any device equipped to display user interfaces, electronic images, and data as described herein. In some implementations, the display device 239 may output display in binary (only two different values for pixels), monochrome (multiple shades of one color), or multiple colors and shades. The display device 239 is coupled to the bus 220 for communication with the processor 235 and the other components of the computing device 200. In some implementations, the display device 239 may be a touch-screen display device capable of receiving input from one or more fingers of a user. For example, the display device 239 may be a capacitive touch-screen display device capable of detecting and interpreting multiple points of contact with the display surface. In some implementations, the computing device 200 (e.g., client device 115) may include a graphics adapter (not shown) for rendering and outputting the images and data for presentation on display device 239. The graphics adapter (not shown) may be a separate processing device including a separate processor and memory (not shown) or may be integrated with the processor 235 and memory 237.

The input/output (I/O) device(s) 247 may include any standard device for inputting or outputting information and may be coupled to the computing device 200 either directly or through intervening I/O controllers. In some implementations, the input device 247 may include one or more peripheral devices. Non-limiting example I/O devices 247 include a touch screen or any other similarly equipped display device equipped to display user interfaces, electronic images, and data as described herein, a touchpad, a keyboard, a scanner, a stylus, an audio reproduction device (e.g., speaker), a microphone array, a barcode reader, an eye gaze tracker, a sip-and-puff device, and any other I/O components for facilitating communication and/or interaction with users. In some implementations, the functionality of the input/output device 247 and the display device 239 may be integrated, and a user of the computing device 200 (e.g., client device 115) may interact with the computing device 200 by contacting a surface of the display device 239 using one or more fingers. For example, the user may interact with an emulated (i.e., virtual or soft) keyboard displayed on the touch-screen display device 239 by using fingers to contact the display in the keyboard regions.

The communication unit 241 is hardware for receiving and transmitting data by linking the processor 235 to the network 105 and other processing systems via signal line 104.

The communication unit 241 receives data such as requests from the client device 115 and transmits the requests to the therapeutic chat interface application 110, for example a request to schedule an appointment with a healthcare provider. The communication unit 241 also transmits information including media to the client device 115 for display, for example, in response to the request. The communication unit 241 is coupled to the bus 220. In some implementations, the communication unit 241 may include a port for direct physical connection to the client device 115 or to another communication channel. For example, the communication unit 241 may include an RJ45 port or similar port for wired communication with the client device 115. In other implementations, the communication unit 241 may include a wireless transceiver (not shown) for exchanging data with the client device 115 or any other communication channel using one or more wireless communication methods, such as IEEE 802.11, IEEE 802.16, Bluetooth® or another suitable wireless communication method.

In yet other implementations, the communication unit 241 may include a cellular communications transceiver for sending and receiving data over a cellular communications network such as via short messaging service (SMS), multimedia messaging service (MMS), hypertext transfer protocol (HTTP), direct data connection, WAP, e-mail or another suitable type of electronic communication. In still other implementations, the communication unit 241 may include a wired port and a wireless transceiver. The communication unit 241 also provides other conventional connections to the network 105 for distribution of files and/or media objects using standard network protocols such as TCP/IP, HTTP, HTTPS, and SMTP as will be understood to those skilled in the art.

The data storage 243 is a non-transitory memory that stores data for providing the functionality described herein. In some implementations, the data storage 243 may be coupled to the components 235, 237, 239, 241, 243, and 247 via the bus 220 to receive and provide access to data. In some implementations, the data storage 243 may store data received from other elements of the system 100 including, for example, entities 135, 140, 145, and/or the therapeutic chat interface applications 110, and may provide data access to these entities. The data storage 243 may store, among other data, user profiles 222, training datasets 224, and machine learning models 226. The data stored in the data storage 243 is described below in more detail.

The data storage 243 may be included in the computing device 200 or in another computing device and/or storage system distinct from but coupled to or accessible by the computing device 200. The data storage 243 may include one or more non-transitory computer-readable mediums for storing the data. In some implementations, the data storage 243 may be incorporated with the memory 237 or may be distinct therefrom. The data storage 243 may be a dynamic random-access memory (DRAM) device, a static random-access memory (SRAM) device, flash memory, or some other memory devices. In some implementations, the data storage 243 may include a database management system (DBMS) operable on the computing device 200. For example, the DBMS could include a structured query language (SQL) DBMS, a NoSQL DMBS, various combinations thereof, etc. In some instances, the DBMS may store data in multi-dimensional tables comprised of rows and columns, and manipulate, e.g., insert, query, update and/or delete, rows of data using programmatic operations. In other implementations, the data storage 243 also may include a non-volatile memory or similar permanent storage device and media including a hard disk drive, a CD-ROM device, a DVD-ROM device, a DVD-RAM device, a DVD-RW device, a flash memory device, or some other mass storage device for storing information on a more permanent basis.

It should be understood that other processors, operating systems, sensors, displays, and physical configurations are possible.

As depicted in FIG. 2, the memory 237 may include the therapeutic chat interface application 110. In some implementations, the therapeutic chat interface application 110 may be configured to implement a secure HTTP API (not shown) to facilitate web, mobile, enterprise, and/or cloud applications for providing patients with access to care for appropriate healthcare services using an intelligent hybrid therapeutic chat interface.

In some implementations, the therapeutic chat interface application 110 may include a user interaction engine 202, a model training engine 204, a therapeutic chat interface engine 206, and a user interface engine 208. The components 202, 204, 206, and 208 may be communicatively coupled by the bus 220 and/or the processor 235 to one another and/or the other components 237, 239, 241, 243, and 247 of the computing device 200 for cooperation and communication. The components 202, 204, 206, and 208 may each include software and/or logic to provide their respective functionality. In some implementations, the components 202, 204, 206, and 208 may each be implemented using programmable or specialized hardware including a field-programmable gate array (FPGA) or an application-specific integrated circuit (ASIC). In some implementations, the components 202, 204, 206, and 208 may each be implemented using a combination of hardware and software executable by the processor 235. In some implementations, each one of the components 202, 204, 206, and 208 may be sets of instructions stored in the memory 237 and configured to be accessible and executable by the processor 235 to provide their acts and/or functionality. In some implementations, the components 202, 204, 206, and 208 may send and receive data, via the communication unit 241, to and from one or more of the client devices 115, the clinician devices 145, the AI hybrid care management server 120, the data sources 135, and third-party servers 140.

The user interaction engine 202 may include software and/or logic to provide functionality for receiving, processing, and storing a stream of user data including interaction data aggregated from one or more entities of the system 100. For example, the user interaction engine 202 may include a series of messages that may be pre-generated to engage with a user accessing a therapeutic chat interface. As another example, the user interaction engine 202 may process a received user input and select from the series of pre-generated messages to respond to the received user input.

In some implementations, the user interaction engine 202 instantiates a data ingestion layer that transports data from assorted data sources 135 to data storage 243 where it can be stored, accessed, and analyzed by the therapeutic chat interface application 110. For example, the data ingestion layer processes incoming data, prioritizes sources, validates individual files, and routes the data to the data storage 243. In some implementations, the user interaction engine 202 instantiates a data transformation layer that maps and converts data from a source format (e.g., of a data source 135) to a destination format. For example, the data transformation layer transforms non-XML data to XML data. The user interaction engine 202 creates a user profile 222 for a user based on processing the received data streams. The user profile 222 may include data and insights about the user including name, unique user identifier, age, gender, interests, height, weight, risk score, location, profile photo, recently measured vital signs, diagnosed conditions (e.g., diabetic, mental health, heart attack, etc.), medical history, user preferences (e.g., phone call for upcoming reminders, etc.), appointment preferences (e.g., video call for virtual urgent care visits, etc.), prescription (e.g., refill dates, etc.), laboratory test results, treatment or care plans, fitness goals (e.g., gain physical mobility, lose weight, etc.), activities (e.g. number of physical therapy sessions, number of missed appointments, synced wearable fitness devices, synced third-party mobile Health applications, etc.), etc. The user interaction engine 202 stores and updates the user profiles 222 in the data storage 243.

The model training engine 204 may include software and/or logic to provide functionality for generating training datasets 224 and training one or more machine learning models 226 or classifiers using the training datasets 224. In some implementations, the model training engine 204 curates one or more training datasets 224 based on the data received and processed in association with a plurality of the client devices 115, the clinician devices 145, the third-party servers 140, and the data sources 135. For example, the model training engine 204 receives the user interaction data, cleans the user interaction data, and derives sample historical and curated text or speech utterance data (words, phrases, sentences, etc.) for generating the training datasets 224. Example training datasets 224 curated by the model training engine 204 may include, but not limited to, a dataset of mental health condition identification hints and patterns, a dataset of user profiles and dispositions for the user profiles, a dataset of medical dictionary terms and indexed search terms, a dataset of clinical context (e.g., past and current health conditions, medications, allergies, laboratory results, treatments, historical encounter data, current encounter data, etc.) for a number of users, etc. The model training engine 204 stores the curated training datasets 224 in the data storage 243. The model training engine 204 uses the training datasets 224 to train the machine learning models for performing the various functionality as described herein. The model training engine 204 stores the trained machine learning models 226 in the data storage 243.

The model training engine 204 creates one or more machine learning models 226 for the therapeutic chat interface engine 206 (described in detail below) to identify intent based on user input. For example, the machine learning model 226 may be a trained natural language understanding (NLU) model that is able to classify user input utterance (e.g., text or speech) and one or more of user profile, past medical and clinical history, user interaction history on different channels, demographic data, etc.

The model training engine 204 facilitates with providing input necessary to create a particular machine learning model 226. In some implementations, the model training engine 204 receives and/or generates data, models, training data, and scoring parameters necessary to create the machine learning model 226. For example, the model training engine 204 may provide curated multilingual text inputs, provide mental health condition identification hints and patterns, provide model negators, perform training, testing, approve, and publish model versions for consumption, perform scoring model parameter tuning, or create scoring accuracy thresholds for generating a model. The model training engine 204 is adapted to receive input from users, such as data scientists, analysts, or operational staff to define and enhance the machine learning models 226. For example, the operational staff may increase or decrease weights for different intent recognitions in the retraining of the machine learning models 226. The model training engine 204 may provide a secure portal through which these users may define, train, test, publish, refine, and improve the machine learning models 226 or introduce new models. For example, the portal may be used to define, train, test and publish NLU models for identification of different intents. The portal allows the users to provide training data-text inputs or spoken word utterances, multi-lingual input, synonyms, conjugation, typos, mispronunciations, model negators, etc. The portal enables the users to define and modify scoring thresholds. The portal allows users to enhance the models during training using machine learning hints, patterns, and/or phrases. The portal further enables the users to control or reduce the overlap of inputs between classes during the training of a machine learning model.

In some implementations, the model training engine 204 emphasizes certain sets of features, traits or attributes in a machine learning model during hyperparameter tuning for improving recognition, accuracy, computational speed, etc. For example, NLU models may be trained based on the following features or attributes, including but not limited to: user profile (age, gender, location, race, socioeconomic, etc.); user clinical context (e.g., past and current health conditions, medications, allergies, laboratory results, treatments, historical encounter data, current encounter data, etc.); medical terms and dictionary; or statistics of input text, predicted disease conditions, proposed and actual dispositions. In some implementations, the model training engine 204 augments or enhances the NLU model with a knowledge base that includes a medical dictionary, index search terms and associated dispositions for those terms. The knowledge base may also be enhanced to include data such as text input matches to text input frequency, disease frequency, drift between prediction and actual disposition, disease severity, user profile (e.g., age, gender, location, language, and other demographic data, etc.), therapist or subject matter expert inputs. The model training engine 204 provides the machine models 226 to the data storage 243 for storage.

In some implementations, the model training engine 204 may be configured to incrementally adapt and train the one or more machine learning models 226 every threshold period of time. For example, the model training engine 204 may incrementally train the machine learning models 226 every hour, every day, every week, every month, etc. based on the aggregated dataset and feedback data generated based on prior predictions made by the machine learning models 226. In some implementations, a machine learning model 226 is a neural network model and includes a layer and/or layers of memory units where memory units each have corresponding weights. A variety of neural network models may be utilized including feed forward neural networks, convolutional neural networks (CNN), recurrent neural networks, radial basis functions, other neural network models, as well as combinations of several neural networks. Additionally, the machine learning model 226 may represent a variety of other machine learning techniques in addition to neural networks, for example, support vector machines, decision trees, Bayesian networks, random decision forests, k-nearest neighbors, linear regression, least squares, hidden Markov models, other machine learning techniques, and/or combinations of machine learning techniques.

In some implementations, the model training engine 204 may train the machine learning model 226 using any one of at least one of supervised learning (e.g., support vector machines, neural networks, logistic regression, linear regression, stacking, gradient boosting, etc.), unsupervised learning (e.g., clustering, neural networks, singular value decomposition, principal component analysis, etc.), or semi-supervised learning (e.g., generative models, transductive support vector machines, etc.). Additionally, or alternatively, the model training engine 204 may train the machine learning model 226 using tensor networks. For example, the model training engine 204 may mine the database to build links across the objects, such as users and their preferences, treatments, outcomes, etc. based on features or attributes that they share with each other in order to train the machine learning models 226.

In some implementations, the model training engine 204 uses keyword-based database lookups, or search by keywords and clinical terms to implement training of one or more machine learning models 226. For example, the model training engine 204 may use keyword extraction for unsupervised training of classification of user input. In some implementations, the model training engine 204 may use transformers and open-source library frameworks for natural language processing, such as spaCy and Spark NLP to build machine learning algorithms to improve intent identification from user input. In some implementations, the model training engine 204 may train one or more machine learning models 226 to perform a single machine learning task or a variety of machine learning tasks. In other implementations, the machine learning model 226 may be trained to perform multiple tasks. In yet other implementations, the model training engine 204 may train a machine learning model 226 to receive the requested data and generate the response data.

The model training engine 204 determines a plurality of training instances or samples from the training dataset 224. The model training engine 204 may apply a training instance as input to a machine learning model 226. The model training engine 204 may generate a predicted machine learning model output by applying training input to the machine learning model 226. Additionally, or alternatively, the model training engine 204 may compare the predicted machine learning model output with a known labelled output from the training instance and, using the comparison, update one or more weights in the machine learning model 226. In some implementations, the model training engine 204 may update the one or more weights by backpropagating the difference over the entire machine learning model 226.

In some implementations, the model training engine 204 may test a trained machine learning model 226 and update it accordingly. The model training engine 204 may partition the training dataset 224 into a testing dataset and a training dataset. The model training engine 204 may apply a testing instance from the training dataset 224 as input to the trained machine learning model 226. A predicted output generated by applying a testing instance to the trained machine learning model 226 may be compared with a known output for the testing instance to update an accuracy value (e.g., an accuracy percentage) for the machine learning model 226. In some implementations, the model training engine 204 may version and service the model 226 through an internal HTTP endpoint to be used by other component(s) of the therapeutic chat interface application 110. For example, once a model 226 is trained and tested and determined to have acceptable accuracy (e.g., accuracy score satisfying a threshold), the model training engine 204 pushes the model 226 to the therapeutic chat interface engine 206 for consumption. In some implementations, model development is an iterative process with retraining, testing and publishing steps performed iteratively, and adapted automatically to improve scores and accuracy. New versions will be published based on improvements and retraining using historical data and efficiency calculations as more data (e.g., feedback) is collected over a period of time. For example, the NLU model or classifier class labels (disease conditions to be identified) requires clinical and business oversight and will be promoted for usage by capability based on clinical and business review. Continuous retraining using training data (utterances, phrases, hints, negators, language variants, etc.) are performed based on curation as part of clinical and data analysis.

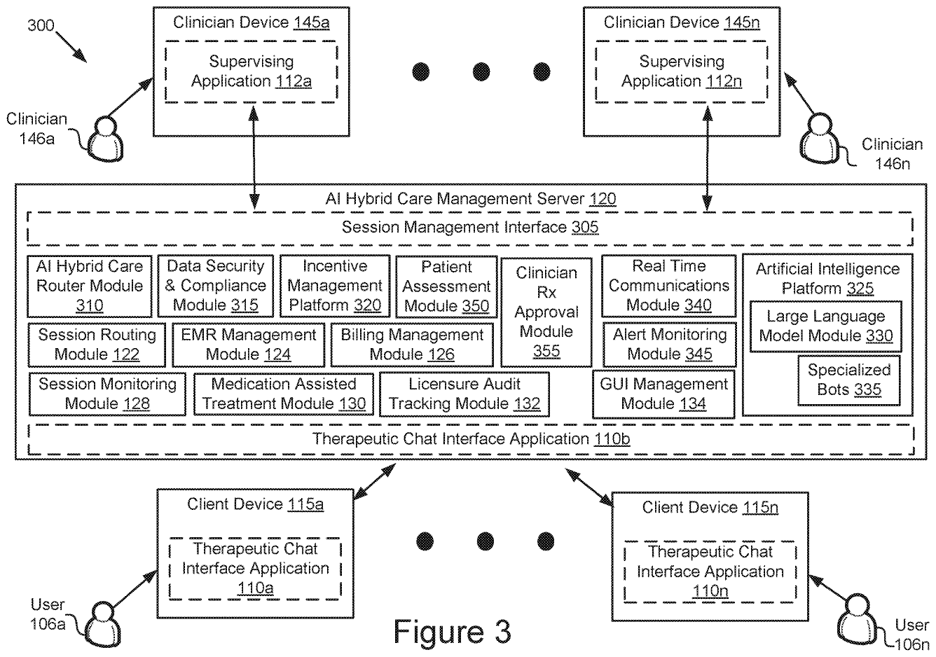

The therapeutic chat interface engine 206 may include software and/or logic to provide functionality for providing a therapeutic chat interface and seamlessly connecting users and clinicians to an AI hybrid care model. For example, the engagement channels may include a website, a mobile application, a phone call, interactive voice response (IVR), instant messaging chat, email, etc. In some implementations, the therapeutic chat interface engine 206 may be communicatively coupled to instances of multiple engagement channels for performing the functionalities as described herein. FIG. 3 is a high-level block diagram illustrating one implementation of an example system 300 for enabling clinicians and users to interact via an intelligent hybrid therapeutic chat interface. As shown in FIG. 3, the therapeutic chat interface is connected to the AI hybrid care management server 12 used by the users and the clinicians provide AI hybrid care to the users. As illustrated, users may be users 106 of client devices 115 operating a therapeutic chat interface application 110. Similarly, clinicians 146 may use clinician devices 145 operating a supervising application 112. Clinicians 146 may monitor the AI agent chatbots generating conversations in the therapeutic chat interface through a supervising application 112 that is communicatively coupled to a session management interface 305 included in the AI hybrid care management server 120 that is backed by the artificial intelligence platform 325 operating on the AI hybrid care management server 120. In an implementation, the artificial intelligence platform 325 includes a large language model module 330. The artificial intelligence platform 325 may also include specialized bots 335 that may be generated, over time, based on various needs. For example, a specialized bot 335 may be generated to orchestrate multiple different requests from a user (e.g., feeling the need to drink after work). As another example, a specialized bot 335 may be generated to handle a specific mental health condition or break an unwanted habit (e.g., a positive interaction at a specific time).

The AI hybrid care management server 120 may enable clinicians and operational staff to enhance the user experience in providing an AI hybrid care model through the session management interface 305 that interacts with one or more components in the AI hybrid care management server 120, in an implementation. For example, the AI hybrid care management server 120 may include an AI hybrid care router module 310, a data security and compliance module 315, an incentive management platform 320, an artificial intelligence platform 325 that includes a large language model module 330 and specialized bots 335, a real time communications module 340, an alert monitoring module 345, and a patient assessment module 350. Additionally, in an implementation, the AI hybrid care management server 120 may include a session routing module 122, an electronic medical record (EMR) management module 124, a billing management module 126, a session monitoring module 128, a medication assisted treatment module 130, a licensure audit tracking module 132, a graphical user interface (GUI) management module 134, and a clinician prescription (Rx) approval module 355.

The clinician Rx approval module 355 may include software and/or logic to provide functionality for enabling a clinician, through a clinician device, to approve or reject a prescription, as well as add new prescriptions, modify existing dosages, change medications, and so forth.

The AI hybrid care router module 310 may include software and/or logic to provide functionality for routing a user to an AI Companion or a human therapist based on the received user responses in a therapeutic chat interface. The platform leverages AI for proactive engagement, crisis detection, and contingency management, with LiveKit enabling real-time communication. LiveKit, a commercially available all-in-one voice artificial intelligence (AI) framework enables AI agents to interact with and “chat” with users in a room, or session. In other embodiments, other voice AI frameworks may be used to provide the same or similar functionality. Therapists intervene as needed, ensuring seamless transitions between AI and human-led sessions. The backend automates workflows, tracks user engagement, and maintains compliance, delivering a scalable and secure hybrid therapy solution.

The data security and compliance module 315 may include software and/or logic to provide functionality for having a secure data and compliant program.

The incentive management platform 320 may include software and/or logic to provide functionality for tracking of incentives provided to users using an Internet-connected database. An offering by which an AI chatbot proactively engages a patient by phone, email, or text to communicate the terms of a contingency management program and to convey ongoing incentives aligned to the patient's participation in care and/or achievement of care goals. “Contingency management” is a term of art in addiction recovery and refers to rewards to a patient to incentivize ongoing engagement with care and progress towards care goals. The incentive management platform may store the available incentives that are provided by a health plan or other healthcare payer relative to specific insurance plans that are underwritten and provided to one or more members, wherein the incentive is provided for engaging an AI Companion for all or part of the care received by the member. The member's participation in the health plan may define “eligibility criteria” for receipt of the incentive. Other eligibility criteria beyond participation in an insurance program may include any number of employer programs; wellness programs; or other programs in which a patient or health plan member may be eligible for the incentives. The storage of this data allows the incentive to be communicated via one or more modalities to the member in order to affect behavior changes towards usage of the AI Companion. The storage of this data also then facilitates tracking of the incentives that are earned by each respective member, and to facilitate payment of the incentive to the member.

In one example, a patient expresses interest in an addiction treatment program. The patient is thereby enrolled in the contingency management program. The AI agent chatbot engages the patient via a modality which may include a phone call with voice-enabled chat, an online or mobile chat, or text message. Although the AI agent may be described as a “chatbot”, the modality of communication may vary and include written text, SMS, voice calls, video chat, phone calls, and other modalities. The AI agent explains the structure of the contingency management program based on the eligibility of that patient relative to stored eligibility criteria, wherein the incentive and eligibility criteria may be defined by the patient's membership in a health plan or other program. In this way, the technology is improved because the incentive and eligibility criteria is retrieved from the member's profile data and integrated into the AI agent, in an implementation.

By way of example, the program may be structured wherein the patient chooses a 2-digit number at intake. A number is generated at random by the healthcare provider each day. If the first digit matches the patient's number that day, they win a small prize (1 in 10 chance). If both digits match, the patient wins a larger prize (1 in 100 chance). The chance to win is conditional on the patient executing elements of their treatment plan (e.g., checking in, performing mindfulness, doing a CBT exercise) and/or achieving treatment goals (e.g., abstaining from substance use). Studies have been conducted that support incentive-based programs as effective in achieving treatment goals.

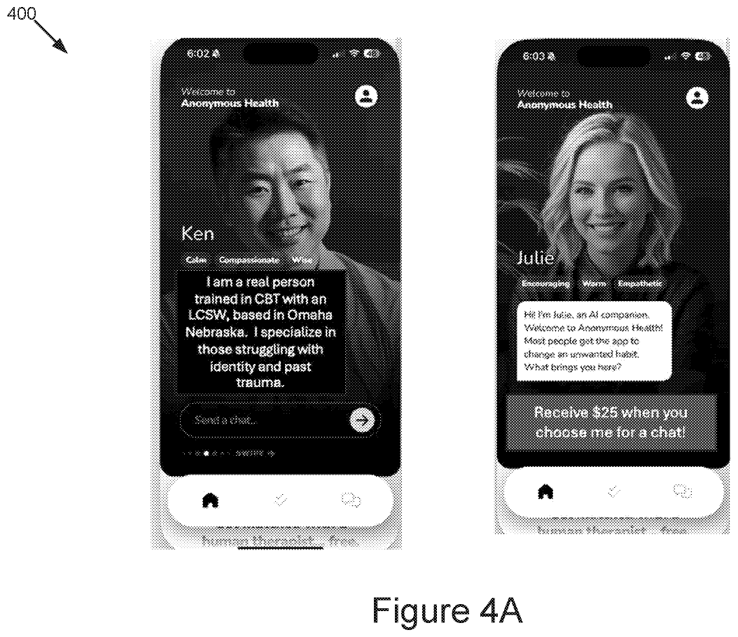

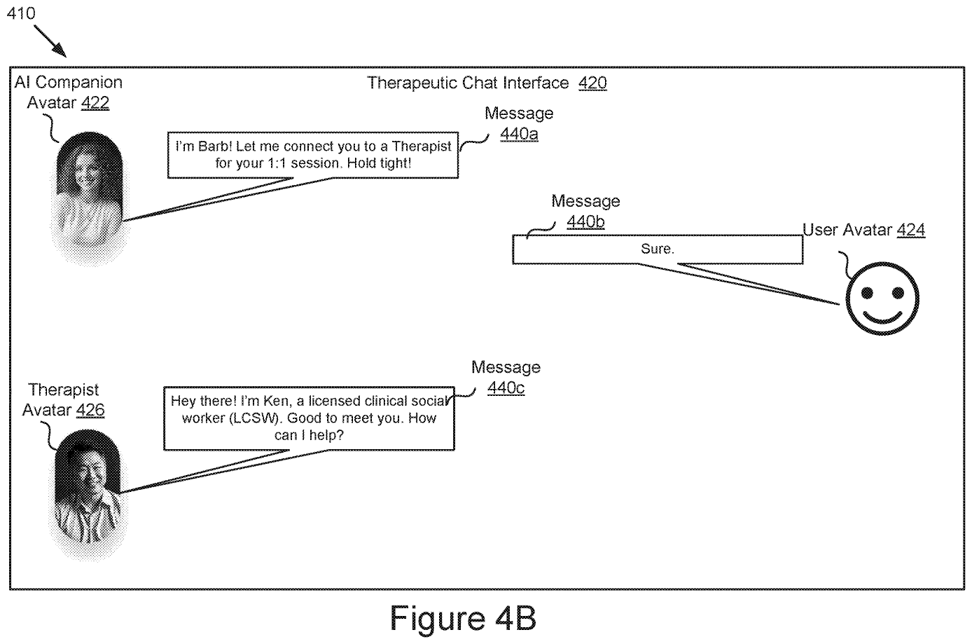

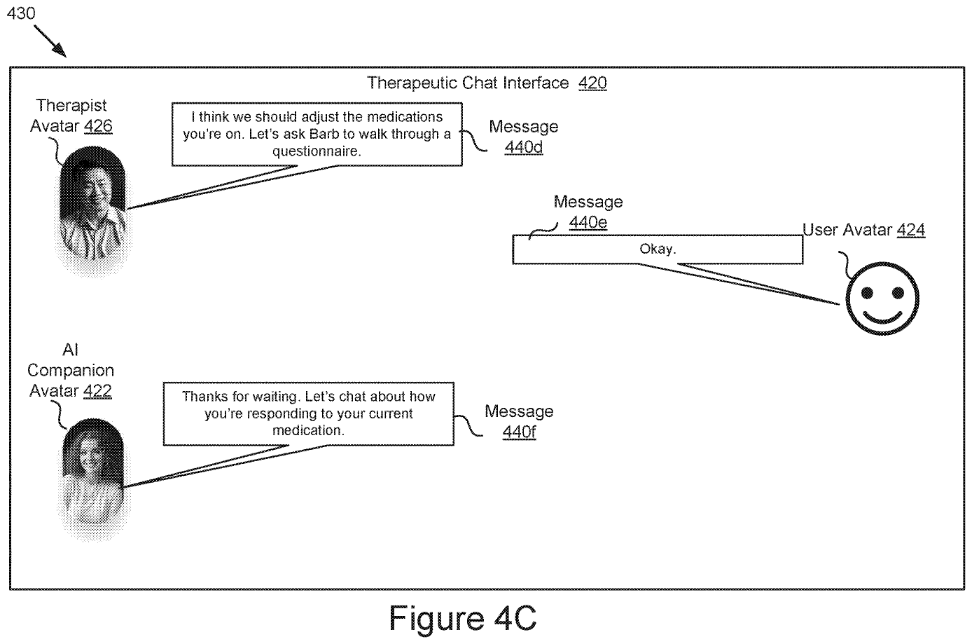

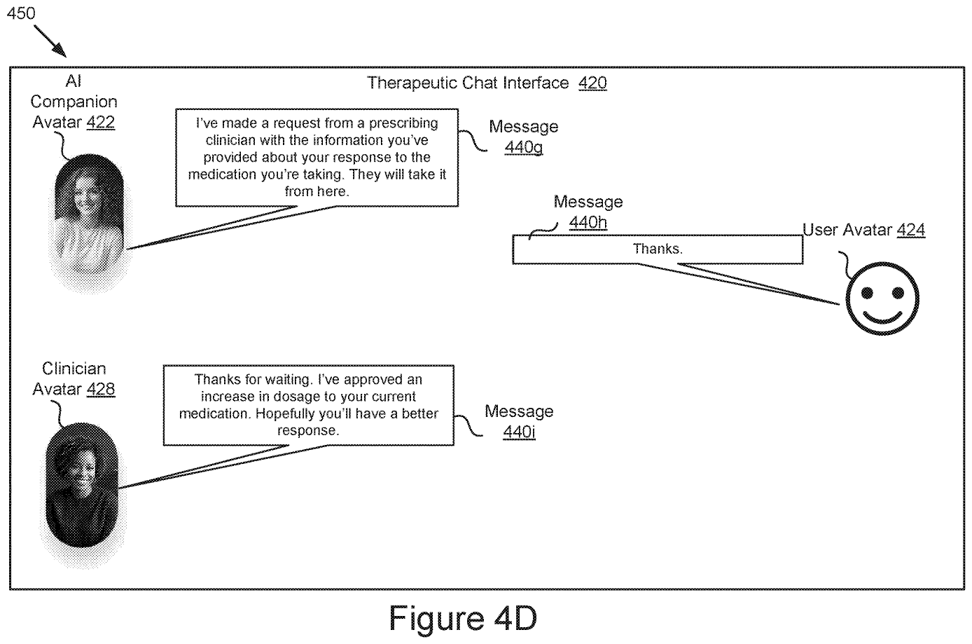

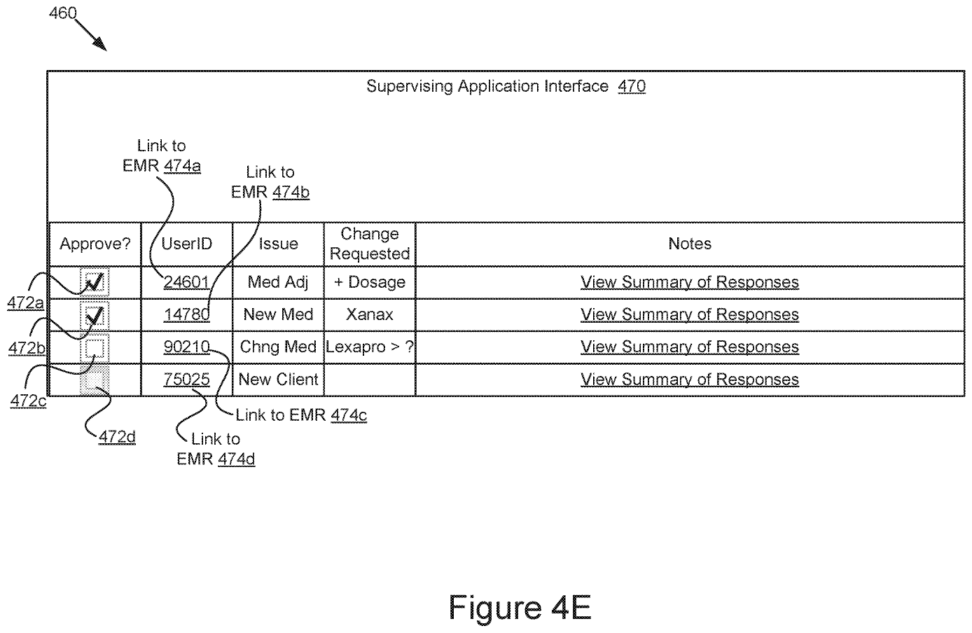

The artificial intelligence platform 325 may include software and/or logic to provide functionality for providing an AI Companion using a large language model module 330 and/or specialized bots 335. FIG. 4A illustrates example user interfaces of a user accessing the therapeutic chat interface to select an AI Companion or a human therapist. A mobile interface is provided for selecting between an AI Companion (Julie) interspersed seamlessly with a human therapist (Ken). This therapeutic chat interface could also be provided on a computer, in another embodiment.

A large language model (LLM), as known to those in the art, may be used to provide the generated text created by the AI Companion with an Internet-connected computer interface. The use of the AI Companion to engage the patient in the program, versus traditional methods of engaging the patient manually in a human-to-human interaction or generating automatic, deterministic messaging, is severalfold: Patients develop a relationship or companionship with the AI Companion that increases retention and engagement. The AI Companion can leverage the interaction associated with contingency management as an opening to a broader chat, wherein the broader chat leverages treatment techniques, such as CBT exercises, to enhance the impact to the patient, or engages in conversation, games, delivery of content, or other engagements tailored to the needs of the individual. This may be informed by data stored in an electronic medical record; data provided by the patient in previous chats; data provided by the patient by virtue of CBT exercises, video consumption; use of tools or techniques, or other engagements in a patient engagement application that is supportive of treatment.

The AI Companion can be prompted to engage the user with the contingency management program at times of known and high risk relative to the patient's triggers or usage patterns. These high risk times may be informed by data stored in an electronic medical record; data provided by the patient in previous chats; data provided by the patient by virtue of CBT exercises, video consumption; use of tools or techniques, or other engagements in a patient engagement application that is supportive of treatment. System and method for driving high, consistent engagement with an AI Companion for those struggling with an addiction or unwanted habit, which may include but are not limited to substance use, digital behaviors, eating behaviors, gambling, or other unwanted habits.

AI Companions, enabled by an AI agent chatbot powered by a trained LLM, can have a very high impact for those struggling with addiction. They can be anonymous, always-available, non-judgmental, trained across a vast evidence-base, and highly personalized. However, it can be a challenge to establish and maintain consistent engagement with the AI Companion. The technical problem of an AI agent chatbot determining how to best engage with a human user through a therapeutic chat interface involves determining how the user best responds to prompts and other stimuli, in an implementation.

This invention discloses a method for establishing and maintaining consistent engagement with an AI agent chatbot to help those struggling with addiction or an unwanted habit. For those struggling with addiction or an unwanted habit, there are often consistent circumstances or “triggers” that prompt usage or engagement of the habit with the patient. An example might be coming home from work; passing a bar or drinking establishment on the way home; getting together with a specific group of people; or any number of other circumstances. These triggers often happen on a recurring pattern including day of week and/or time of day.

Programming an AI agent chatbot to engage in a highly interactive and compelling way at the times of these triggers can be a highly effective way of diverting from the usage.

Moreover, in the first or early engagement between a patient and an AI agent chatbot, it can be a highly effective and predictable way to: (1) initiate a relationship, (2) establish value of the AI agent chatbot to the patient, and (3) actuate an effective intervention by inquiring about the triggers, inquiring about the temporal pattern of the triggers, and then establishing a proactive outreach and engagement cadence according to that temporal pattern.

An example initial conversation may include:
AI Companion: “Hi, I'm Julie! Great to meet you.”
AI Companion: “What brought you here?”
++++++++++++++++
User: I'm trying to cut back on drinking.
+++++++++++++++++
AI Companion: “Good for you! Why did you decide to do that?”
++++++++++++++
User: I've been drinking way too much ever since I lost my job. My wife says she's going to leave me.
++++++++++++++
AI Companion: “Ok. What time of day do you typically start drinking?”
++++++++++++++
User: I drink after work every day. I get home at around 5 μm.
++++++++++++++
AI Companion: “Would it help you if I reach out to you every day at 5 μm? I can try to help out! That's a good time for us to chat, and I can take your mind off things.;-)”
++++++++++++
User: Yes that would be really helpful
++++++++++++
AI Companion: “Ok, I'm going to do that!”
AI Companion: “When I send you the note, try to find time to get on the app and chat with me. It will be fun and I can help out.”
+++++++++++++++++++++++++=
FOLLOW-ON CONVERSATIONS

Once the relationship and temporal pattern has been established, the AI Companion can proactively engage the user via any number of modalities (e.g., voice, phone call, push notifications, text message) to engage the user at times of high risk. Engagements may be designed to divert attention, create fun, or otherwise generate value for the user. They also might be paired with contingency management updates per the contingency management inventions disclosed herein.

Examples of proactive outreach “hooks” that may be generated from the AI agent chatbot as few shot examples to in turn inform the AI generated \ engagement with the patient at the time(s) of high risk.

### Proactive Companion Outreach: First 10 Days

AI Companion: “Hey there! You asked me to reach out this time of day, and here I am! What are you up to?;-)”
“Me again! Is now still a good time? Want to hear something funny?”
“Oof. You wouldn't believe the story I just heard.”
“I've got a game for us. Ready?”
“You're in my app! Today, I'm going to unlock something new for you.;-)”
“Are you keeping secrets from me!?!?! You tell me yours and I'll tell you mine!”
“Ok, I've got a bet for you.”
   Upon engagement→“I bet I can make you feel better in 1 minutes. Do you believe me?
   Ok, but you have to do exactly what I say! Do you promise?;-)”

AI Companion executes whole mindfulness exercise in line in his/her voice—3 deep breaths.
“I want to get to know you better! Tell me something about you. Who was your best friend growing up? What were you like as a kid? I want to hear!”
“Somebody just told me they want to speak with a real person instead of me! Which I understand. Do you ever feel that way?”

### First Friday Override

“Yay, it's a Friday! Want to plan something?”

The real time communications module 340 may include software and/or logic to provide functionality for providing communications through text, audio, and video technologies through the therapeutic chat interface. The system follows a hybrid AI-human therapy model, where an AI chatbot provides continuous support while seamlessly integrating human therapists when necessary. The LiveKit-based architecture enables real-time communication, ensuring an effective and responsive support system. Core components include an AI Companion (LLM-Powered Chatbot) that provides 24/7 engagement, companionship, and contingency management. The AI Companion may be configured to proactively reach out to users based on behavioral triggers. Additionally, the AI Companion may be further configured to detect crisis moments using AI (LLM) and escalate to human therapists. Another component of the architecture is human therapist integration. This enables seamless transitions from AI chat to live sessions with therapists. Additionally, hybrid care sessions are also enabled, blending AI-led engagement with human therapy.

Contingency management & incentives are another core component of the architecture. By monitoring user progress and triggering reward mechanisms, users are incentivized to continue engaging with the therapeutic chat interface. Gamification elements, such as a daily lottery and milestone tracking, may be used by the incentive management platform.

LiveKit Integration for Real-Time Interaction. LiveKit powers the real-time communication layer of your architecture: AI-Driven Session Routing determines whether a session remains AI-driven or escalates to a therapist. Users are routed to therapists based on availability and need.

A platform is provided for voice & video communication that facilitates smooth transitions from text-based AI chat to video/audio therapy and ensures low-latency, HIPAA-compliant interactions.

Further, automated monitoring & quality assurance is also provided through the same platform. An AI agent moderates conversations and flags urgent cases for human intervention. Sessions are recorded and analyzed for compliance and effectiveness.

The backend operations include a data flow, using technologies such as DynamoDB & Vector Search (OpenSearch/Pinecone). Session history and user interactions are stored in a database. Personalized recommendations and interventions are powered through the techniques and methods described above. In an embodiment, a temporal workflow engine is used to automate engagement sequences and contingency management and align AI outreach with user behavioral patterns. Additionally, sensitive data is secured through secure API & identity management using technologies such as Amazon Web Services (AWS) Cognito that handles authentication and identity management to ensure HIPAA-compliant data storage and encryption.

The alert monitoring module 345 may include software and/or logic to provide functionality for automatically escalating a situation to human therapists based on user interactions in a therapeutic chat interface.

The patient assessment module 350 may include software and/or logic to provide functionality for using artificial intelligence models to determine if a patient, through their behaviors and/or user interactions, requires an intervention based on an assessment.

The therapeutic chat interface engine 206 identifies a user on an engagement channel requesting digital health care and determines a contextual state of the user based on their history of prior interactions across different engagement channels. For example, a user may have engaged in a long conversation via a phone call with an AI agent chatbot a day before. When the user engages for AI hybrid care via instant messaging chat the next day, the therapeutic chat interface engine 206 is contextually aware that the user engaged in a phone conversation and has access to a transcript of the conversation. The therapeutic chat interface engine 206 receives user input in association with a user on one or more engagement channels. For example, the user input may be received in association with an online booking of an appointment for a user with a human therapist during a temporal workflow that indicated that a situation should be escalated.

The user input may be one or more of text, speech, voice to text, image, and video. For example, the user input may include a trigger spoken by the user during the phone conversation the day before and the user input may further include a user generated text input that indicates an emergency. The therapeutic chat interface engine 206 provides support for escalating the situation to a human therapist. In some implementations, the therapeutic chat interface engine 206 receives a stream of conversational text and/or speech for stream processing. In other implementations, the therapeutic chat interface engine 206 receives a batch of conversational text and/or speech for batch processing. The therapeutic chat interface engine 206 performs real time speech to text translation and text to speech translation using one or more of language models, transcription models, and voice recognition models.

The therapeutic chat interface engine 206 is coupled to receive one or more operational machine learning models 226 deployed by the model training engine 204 for identifying intent in the user input, surfacing questionnaire and processing answers to invoke linked healthcare services for the user. The therapeutic chat interface engine 206 provides natural language understanding of intent based on user input. The therapeutic chat interface engine 206 processes the user input using one or more machine learning models 226 (e.g., NLU models) to understand natural language (e.g., English, Spanish, German, etc.) of the user input (e.g., textual words or phrases) and identify the intent (e.g., potential health or disease condition). In addition to the user input, the therapeutic chat interface engine 206 may receive and process profile data (e.g., age, gender, demographic data, etc.), clinical data (e.g., past medical history, EHR, etc.), pharmacy data (e.g., medication adherence, etc.) of the user using the machine learning model. For example, during the online booking of an appointment, if a user indicates they are experiencing dizziness, the therapeutic chat interface engine 206 interprets the user's natural language with a NLU model to infer the patient's condition is a red flag condition requiring immediate care based on the user's demographic profile and medical history.

The user interface engine 208 may include software and/or logic for providing user interfaces to a user. In some implementations, the user interface engine 208 receives instructions from the components 202, 204, and 206, generates a user interface according to the instructions, and transmits the user interface for display on the client device 115. In some implementations, the user interface engine 208 sends graphical user interface data to an application (e.g., a browser) in the client device 115 via the communication unit 241 causing the application to display the data as a graphical user interface.

As depicted in FIG. 2, the memory 237 may also include the supervising application 112. In some implementations, the supervising application 112 may be configured to implement a secure HTTP API (not shown) to facilitate web, mobile, enterprise, and/or cloud applications for providing clinicians with access to the AI hybrid care provided through the intelligent hybrid therapeutic chat interface on the therapeutic chat interface application 110.

In some implementations, the supervising application 112 may include a supervising user interactions module 210, an override chat interface engine 212, and a user incentive engine 214. The components 210, 212, and 214 may be communicatively coupled by the bus 220 and/or the processor 235 to one another and/or the other components 237, 239, 241, 243, and 247 of the computing device 200 for cooperation and communication. The components 210, 212, and 214 may each include software and/or logic to provide their respective functionality. In some implementations, the components 210, 212, and 214 may each be implemented using programmable or specialized hardware including a field-programmable gate array (FPGA) or an application-specific integrated circuit (ASIC). In some implementations, the components 210, 212, and 214 may each be implemented using a combination of hardware and software executable by the processor 235. In some implementations, each one of the components 210, 212, and 214 may be sets of instructions stored in the memory 237 and configured to be accessible and executable by the processor 235 to provide their acts and/or functionality. In some implementations, the components 210, 212, and 214 may send and receive data, via the communication unit 241, to and from one or more of the client devices 115, the clinician devices 145, the AI hybrid care management server 120, the data sources 135, and third-party servers 140.

The supervising user interactions module 210 may include software and/or logic to provide functionality for monitoring user interaction data aggregated from one or more entities of the system 100. In an implementation, user interaction data may be aggregated and summarized by an AI agent chatbot to generate a summary of user interactions over a period of time. In another implementation, certain user interactions may be flagged based on business logic rules that may be configured by the supervising user. For example, suicidal ideations may be a criteria or business logic rule that indicates the user is experiencing a mental health emergency requiring immediate intervention.

The override chat interface engine 212 may include software and/or logic to provide functionality for providing clinicians the ability to override chats in a therapeutic chat interface in one or more entities of the system 100. For example, a clinician may be monitoring user interactions with an AI Companion through the supervising user interactions module 210 and determine that an intervention is warranted. Thus, the override chat interface engine 212 may enable the clinician to join a conversation, or “join a call” to override chats in a therapeutic chat interface.

The user incentive engine 214 may include software and/or logic to provide functionality for providing users with an incentive to use a therapeutic chat interface in one or more entities of the system 100. As discussed herein, a monetary incentive may be delivered to a user for engaging with the therapeutic user interface and the AI Companion to complete various tasks and/or CBT exercises that aid in mental health. Various schemes and structures may be used to provide users with an incentive to use the therapeutic chat interface in one or more entities of the system 100. In an implementation, an AI model may be generated to optimize the incentive schemes based on the individual preferences and/or needs of a user.

The session routing module 122 may include software and/or logic to provide functionality for providing a session routing to move information from one entity to another, seamlessly. For example, the different clinicians may be combined in one chat interface to provide a singular experience to the user, integrating a conversation with a human therapist, a conversation with a medical practitioner (e.g., licensed nurse, doctor, or other medical professional), and an AI Companion/AI chatbot. The session routing module 122 enables the conversations to flow dynamically.

The electronic medical record (EMR) management module 124 may include software and/or logic to provide functionality for tracking services rendered to the user, including medication assisted treatment, talk therapy, and medical consultations. For example, a number of hours of talk therapy may be recorded as services rendered to the user based on user interactions with the AI Companion and/or therapist, in an implementation. An electronic medical record (EMR) may be stored in a format that is accessible to other systems, such as insurance billing systems, healthcare systems, and/or hospital systems. In another embodiment, a detailed record of each medical consultation, such as a session with a nurse practitioner via a video visit, may be stored in the electronic medical record for billing purposes for an insurance provider. For example, a therapist may determine that the user should be placed on a new psychotropic medication to treat a diagnosis of clinical depression and connect the user, through the therapeutic chat interface, to an available psychiatrist or other medical practitioner who is licensed to prescribe the medication. The video visit with the medical practitioner may last 10-15 minutes based on the previous user interactions with the AI Companion and therapist, for example. Because the coordination of care is nearly instantaneous, the user experience is greatly improved. Furthermore, the user's electronic medical record and/or summary of user interactions with the AI Companion and clinician may be made available to a medical practitioner in a simple therapeutic chat interface, resulting in faster processing of user data through one or more systems that help deliver patient care. This improves the efficiency of data transfer of the user's information through these systems based on the therapeutic chat interface application 110.

The billing management module 126 may include software and/or logic to provide functionality for integrated and automated billing management for generating a billing invoice for insurers and/or the user. For example, an offer to start new medication may be recommended by a therapist in a one-on-one conversation, but the user may not accept the offer. Thus, the services for medication assistance may automatically be removed from a bill. In other embodiments, a single bill may include the past month's services rendered through the therapeutic chat interface.

The session monitoring module 128 may include software and/or logic to provide functionality for monitoring the sessions generated by users. For example, a session may be monitored and parsed after a conversation has ended to determine various next actions that should be triggered, such as making a request to schedule a therapy appointment with a human therapist. As another example, the session monitoring module 128 may include an AI parser or other system reading through a chat conversation and identifying next actions to queue in a workflow to be performed by another module in the AI hybrid care management server 120.

The medication assisted treatment module 130 may include software and/or logic to provide functionality for providing a protocol for an AI Companion to follow in gathering information needed to prescribe a medication for treatment of the user. For example, different prescription medications have different protocols, such as checklists, that include various questions to ask of a user. One medication, used for clinical depression, may have unwanted side effects that could have interactions with other medications that the user is taking. Additionally, a protocol may be delivered by the AI Companion to gather information, but the medical practitioner may be required to sign off or otherwise acknowledge that the protocol has been followed. Thus, the medication assisted treatment module 130 may include various required prompts that may require multiple signoffs to comply with legal requirements and/or licensure obligations.

The licensure audit tracking module 132 may include software and/or logic to provide functionality for generating audit trail and/or log information for the licenses being used for one or more portions of a conversation in the therapeutic chat interface with the user. For example, a portion of the conversation may be covered by a licensed clinical social worker where another portion of the conversation may be covered by a licensed nurse practitioner. The licensure audit tracking module 132 may include the timestamps and user identifying information of the clinicians that are associated with each portion of the conversation with the user.

The graphical user interface (GUI) management module 134 may include software and/or logic to provide functionality for generating different GUIs and/or avatars that are presented to the user based on different contexts. For example, the one-on-one therapy session may have a different GUI than the chat interface with the AI Companion. During the medication assisted treatment portion of the session, another GUI may be presented with a different avatar representing the nurse practitioner, for example.

FIGS. 4B-4D are graphical representations of example data structures used to provide a user accessing the therapeutic chat interface an example incentive. For example, FIG. 4B illustrates an example screen 410 showing the therapeutic chat interface 420 that includes an AI Companion avatar 422 and a message 440a to the user, represented by user avatar 424. FIG. 4B illustrates an example handoff and seamless integration of different conversations. Message 440b from the user indicates an acceptance of the handoff to the therapist, represented by therapist avatar 426. Message 440c includes identifying information of the therapist, including their licensure credentials.

FIG. 4C illustrates an example screen 430 that includes the therapeutic chat interface 420 as updated with new messages 440. For example, the therapist indicates in message 440d that an adjustment to the medications that the user is taking may be needed. Message 440e from the user indicates acceptance of initiating a conversation for adjusting the medication. Then, the AI Companion, represented by the avatar 422, initiates a protocol or questionnaire associated with adjusting the medication that the user is taking in message 440f.

FIG. 4D illustrates an example screen 450 that includes the therapeutic chat interface 420 as updated with new messages 440. Here, another handoff is illustrated, where the AI Companion has gathered the necessary information for adjusting the medication and making a request for a prescribing clinician with the gathered information as indicated in message 440g. The user replies with “Thanks” in message 440h, indicating that the user has acknowledged the request. At some time later, a clinician may review the information on their clinician device and either approve or deny the request. In message 440i, the clinician, as represented by clinician avatar 428, indicates that the request to increase the dosage has been approved.

FIG. 4E is an example graphical representation of an example user interface for clinicians to approve medication assisted treatment as coordinated through the intelligent hybrid therapeutic chat interface. As shown in screen 460, a supervising application interface 470 includes a table with selectable user interface elements, including toggle checkboxes 472, links 474 to electronic medical records of users assigned to the clinician, a brief description of the issue, an indication of the change to medication requested, and notes that are linked to the user responses in the therapeutic chat interface 420. In other embodiments, more or less information may be provided in the supervising application interface 470.

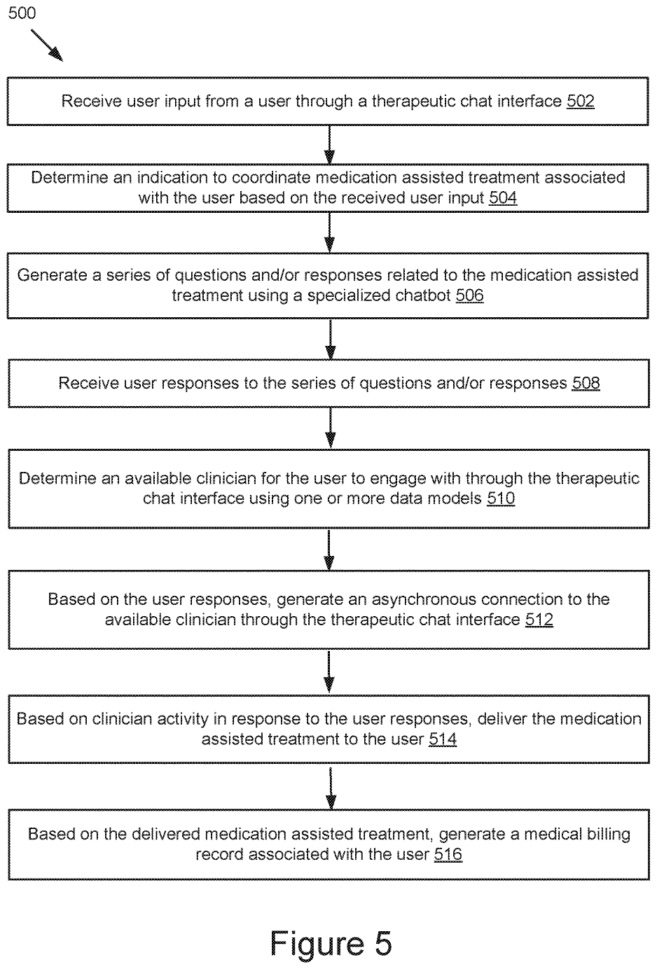

FIG. 5 is an example flowchart diagram of a method of generating a billing record associated with a user to based on delivering medication assisted treatment through a therapeutic chat interface for mental health-related issues. Method 500 may begin with user input being received 502 from a user through a therapeutic chat interface. For example, a user may operate a mobile application to enter text, such as “I want better meds.” As another example, the mobile application may enable omnichannel support, such as telephony interface to call an interactive voice response (IVR) chatbot. Additionally, different channels of communication can be utilized and orchestrated, enabling different types of user input to be received from the different channels. For example, in a situation where the user enters user input regarding a need to chat with the AI Companion and a request to talk to a human therapist about modifying their medication treatment, the two items may be prioritized and orchestrated separately.

An indication to coordinate medication assisted treatment associated with the user is determined 504 based on the received user input. Using one or more natural language processing (NLP) models, the indication to coordinate medication assisted treatment associated with the user may be determined 504. In this way, the therapeutic chat interface understands the intent of the user to seek access to a medical practitioner to assist with prescribing medication for the determined one or more mental health issues. A series of questions and/or responses related to the medication assisted treatment are generated 506 using a specialized AI chatbot. For example, additional questions related to a determined mental health issue of “I want better meds” may be retrieved from a data store of questions, such as “Ok, I can help with that” and “I can coordinate with one of our clinicians in helping adjust your medication.” User responses to the series of questions and/or responses are then received 508. An available clinician for the user to engage with through the therapeutic chat interface is determined 510 using one or more data models. For example, the conversation may be parsed so that the indication to coordinate medication assisted treatment may be linked to a set of available clinicians who are licensed to prescribe medications in the treatment of the user. The clinicians may be “available” in the sense that they are able, within a set period of time, to respond to the request and have capacity to handle the request.

Based on the user responses, an asynchronous connection to a clinician device may be generated 512 through the therapeutic chat interface. As described above, an AI-powered monitoring system may determine, based on the user responses and/or other information accessible to the AI hybrid care management server 120, that the situation should be escalated, causing the asynchronous connection to the available clinician to be generated 512 through the therapeutic chat interface. Then, based on clinician activity in response to the user responses, the medication assisted treatment may be delivered 514 to the user. Lastly, based on the delivered medication assisted treatment, a medical billing record may be generated 516 associated with the user.

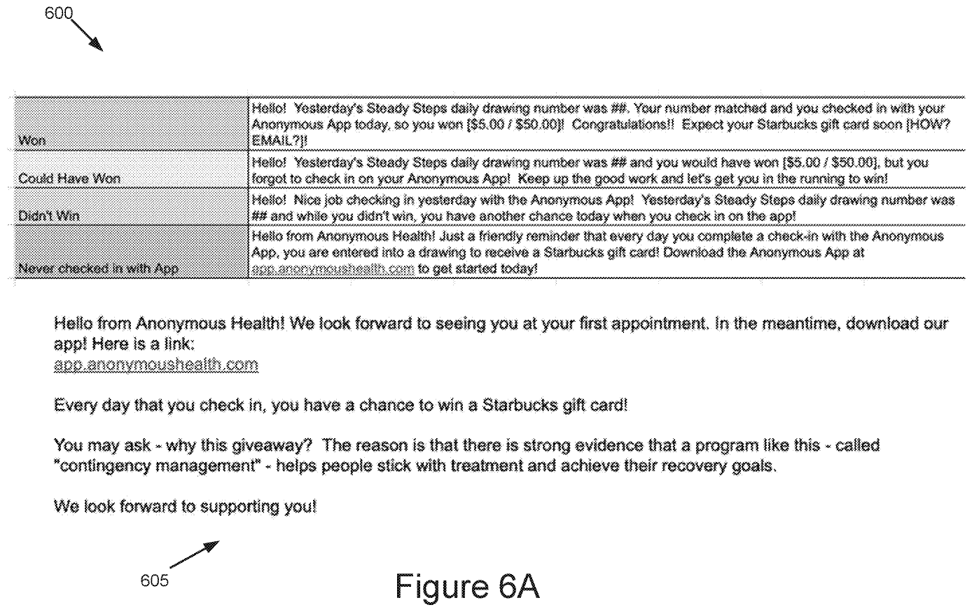

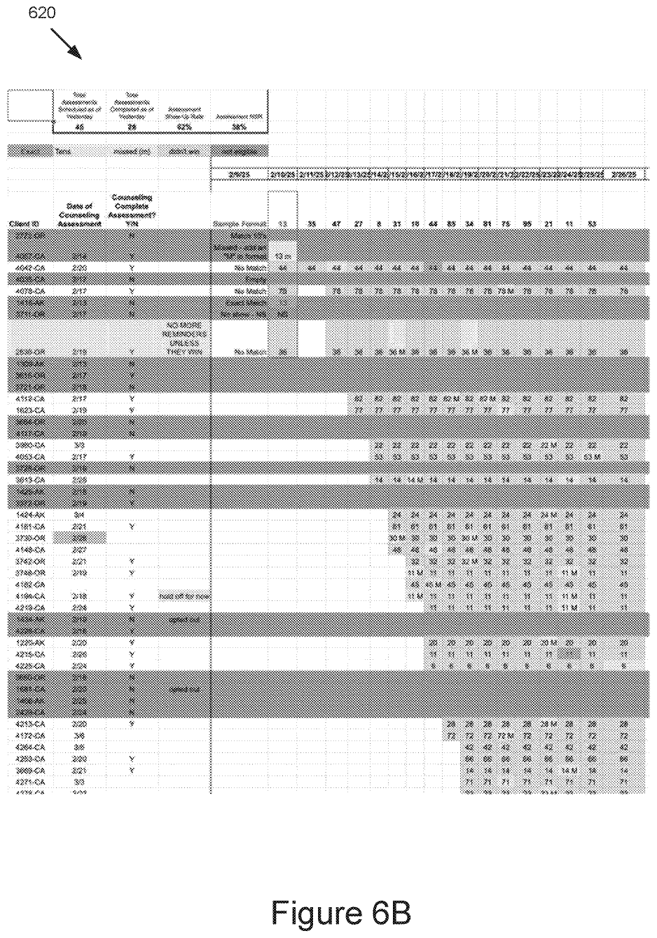

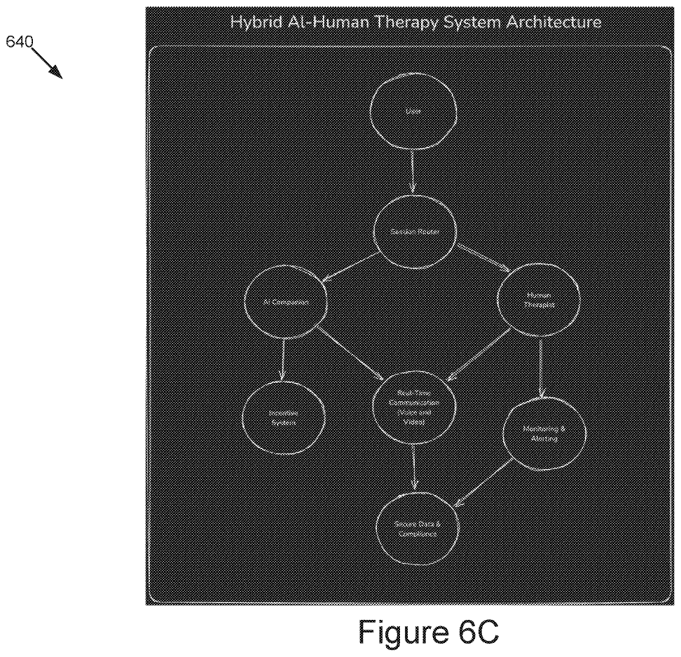

FIGS. 6A-6C graphical representations of example data structures used to provide a user accessing the therapeutic chat interface an example incentive, in accordance with an implementation. FIG. 6A illustrates example messages that may be delivered to users to encourage them to keep using the therapeutic chat interface through the incentive management platform 320. For example, a short message 605 may be delivered to a user through the incentive management platform 320. FIG. 6A also illustrates other example messages 600 that may be delivered based on the contingency management program described above. Messages 600 include example engagements from the AI Companions that may shape its outreach under various scenarios for a daily lottery which determines whether or not the patient receives the reward.

FIG. 6B includes an example data structure used to track incentives for users to continue using the therapeutic chat interface. A table 620 illustrates how a program can be executed and tracked. This tracking can be done automatically with a relational database and programming, and the results can trigger and prompt the AI Companion chatbot to engage, wherein the prompt leverages few short example correspondences.

In FIG. 6C, an example high-level block diagram illustrates one implementation of an example system for providing users with access to care using an intelligent hybrid therapeutic chat interface. An example architecture and enablement overview is displayed in FIG. 6C at diagram 640. The system follows a hybrid AI-human therapy model, where an AI chatbot provides continuous support while seamlessly integrating human therapists when necessary. In another implementation, a human therapist may provide continuous support while activating the AI chatbot when helpful or necessary, preferably instructing the AI chatbot on one or more content directions for which it will engage the patient, such as Cognitive Behavioral Therapy (CBT) exercises, coping exercises, clinical screeners, outcomes data collection, appointment scheduling, and the like. The LiveKit-based architecture enables real-time communication, ensuring an effective and responsive support system. Core components include an AI Companion (LLM-Powered Chatbot) that provides 24/7 engagement, companionship, and contingency management. The AI Companion may be configured to proactively reach out to users based on behavioral triggers. Additionally, the AI Companion may be further configured to detect crisis moments using AI (LLM) and escalate to human therapists. Another component of the architecture is human therapist integration. This enables seamless transitions from AI chat to live sessions with therapists. Additionally, hybrid care sessions are also enabled, blending AI-led engagement with human therapy. In this way, hybrid care sessions improve the existing technology, providing historical data to improve one or more AI data models and/or machine learning models that the AI Companion uses to produce generated content.

Contingency management & incentives are another core component of the architecture shown in FIG. 6C. By monitoring user progress and triggering reward mechanisms, users are incentivized to continue engaging with the therapeutic chat interface. Gamification elements, such as a daily lottery and milestone tracking, may be used by the incentive management platform.

LiveKit Integration for Real-Time Interaction. LiveKit powers the real-time communication layer of your architecture: AI-Driven Session Routing determines whether a session remains AI-driven or escalates to a therapist. Users are routed to therapists based on availability and need.

A platform is provided for voice & video communication that facilitates smooth transitions from text-based AI chat to video/audio therapy and ensures low-latency, HIPAA-compliant interactions.

Further, automated monitoring & quality assurance is also provided through the same platform. An AI agent moderates conversations and flags urgent cases for human intervention. Sessions are recorded and analyzed for compliance and effectiveness.

The backend operations include a data flow shown in FIG. 6C, using technologies such as DynamoDB & Vector Search (OpenSearch/Pinecone). Session history and user interactions are stored in a database. Personalized recommendations and interventions are powered through the techniques and methods described above. In an embodiment, a temporal workflow engine is used to automate engagement sequences and contingency management and align AI outreach with user behavioral patterns. Additionally, sensitive data is secured through secure API & identity management using technologies such as Amazon Web Services (AWS) Cognito that handles authentication and identity management to ensure HIPAA-compliant data storage and encryption.

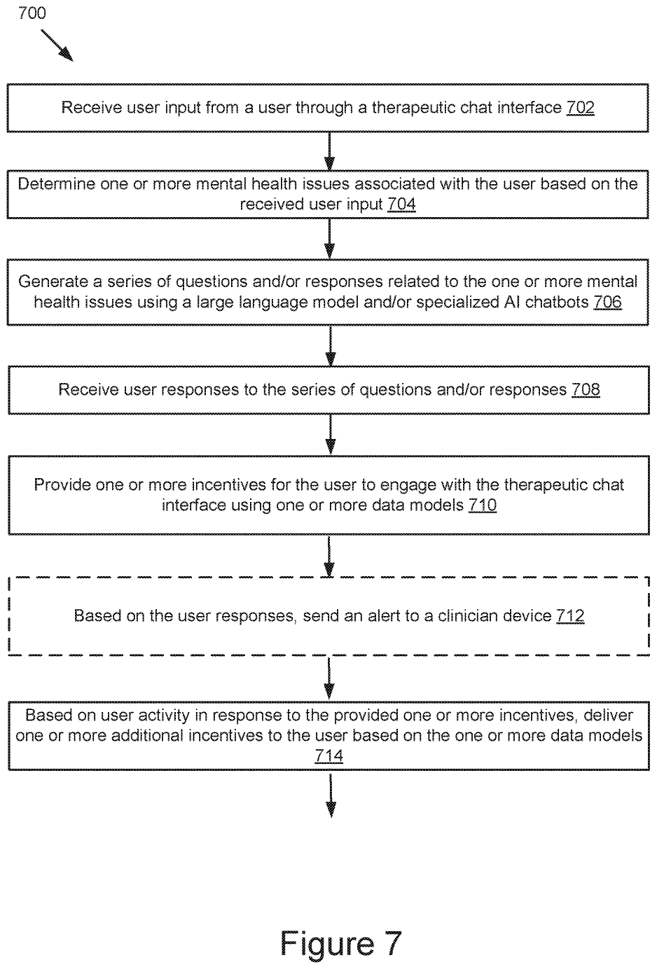

FIG. 7 is an example flowchart diagram of a method of incentivizing a user to access the therapeutic chat interface for mental health-related issues. Method 700 may begin with user input being received 702 from a user through a therapeutic chat interface. For example, a user may operate a mobile application to enter text, such as “I want a drink.” As another example, the mobile application may enable omnichannel support, such as telephony interface to call an interactive voice response (IVR) chatbot. Additionally, different channels of communication can be utilized and orchestrated, enabling different types of user input to be received from the different channels. For example, in a situation where the user enters user input regarding a need to chat with the AI Companion and a request to talk to a human therapist about a trigger to drink after work, the two items may be prioritized and orchestrated separately.

One or more mental health issues associated with the user is determined 704 based on the received user input. Using one or more natural language processing (NLP) models, the one or more mental health issues may be determined 704. In this way, the therapeutic chat interface understands the intent of the user to seek access to mental health care for the determined one or more mental health issues. A series of questions and/or responses related to the one or more health issues are generated 706 using a large language model and/or specialized AI chatbots. For example, additional questions related to a determined mental health issue of “I want a drink” may be retrieved from a data store of questions, such as “Ok, I've got a bet for you” and “I bet I can make you feel better in 1 minutes. Want to give it a try?” as described above. User responses to the series of questions and/or responses are then received 708. One or more incentives for the user to with the therapeutic chat interface are provided 710 using one or more data models. For example, the response to the question regarding “Do you believe me?” regarding the unwanted habit of drinking after work of “Yes” may be linked to a particular incentive or gamification engagement program.

Based on the user responses, an alert may be sent 712 to a clinician device, optionally. As described above, an AI-powered monitoring system may determine, based on the user responses and/or other information accessible to the AI hybrid care management server 120, that the situation should be escalated, causing an alert to be sent 712 to a clinician device. Lastly, based on user activity in response to the provided one or more incentives, one or more additional incentives may be delivered 714 to the user based on one or more data models. For example, as shown in FIG. 6B, the one or more data models may indicate that the user activity in response to the provided one or more incentives may warrant an additional incentive to continue usage of the therapeutic chat interface by the user.

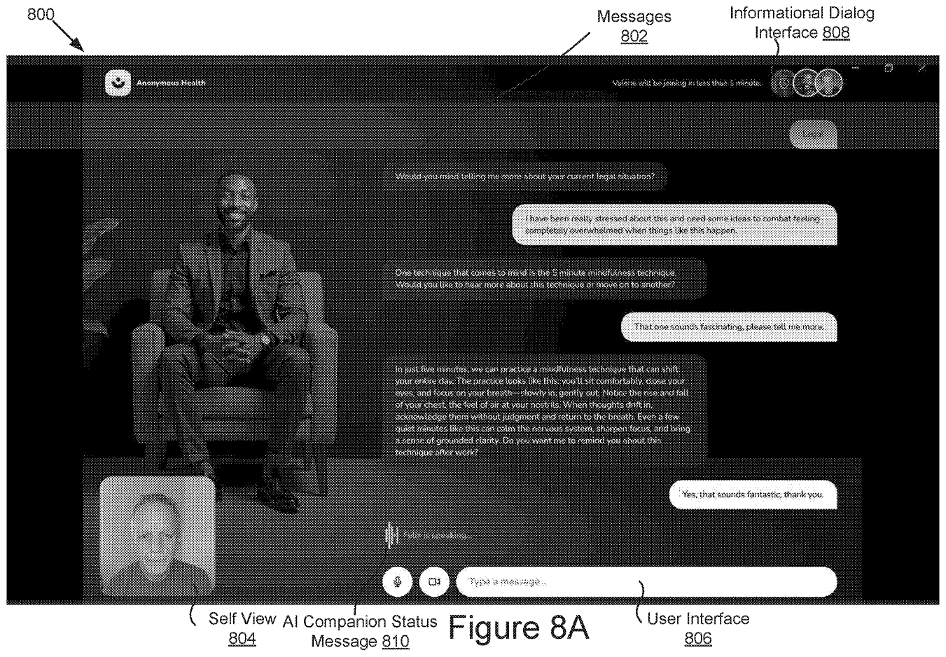

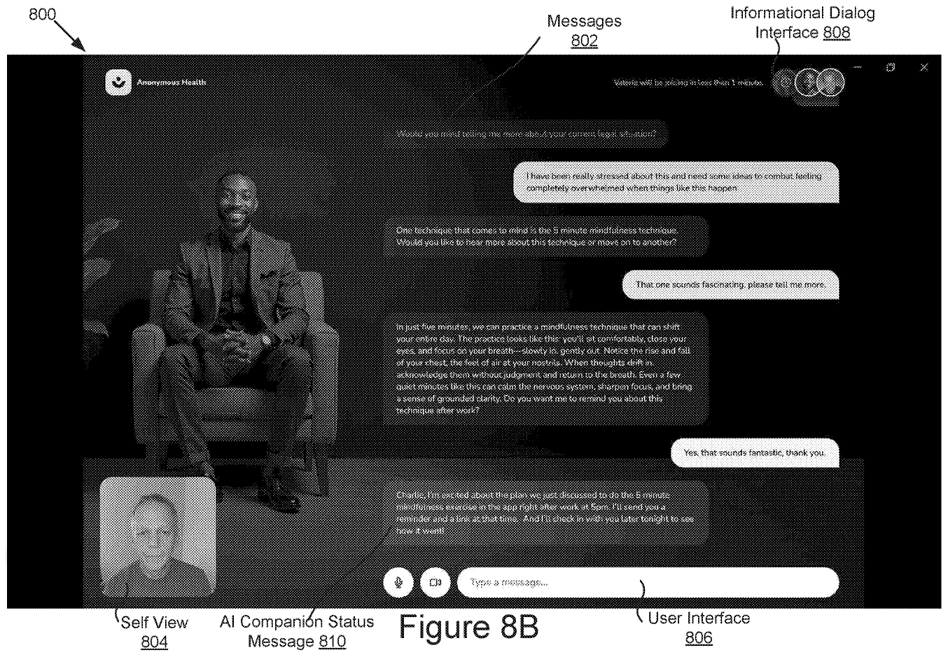

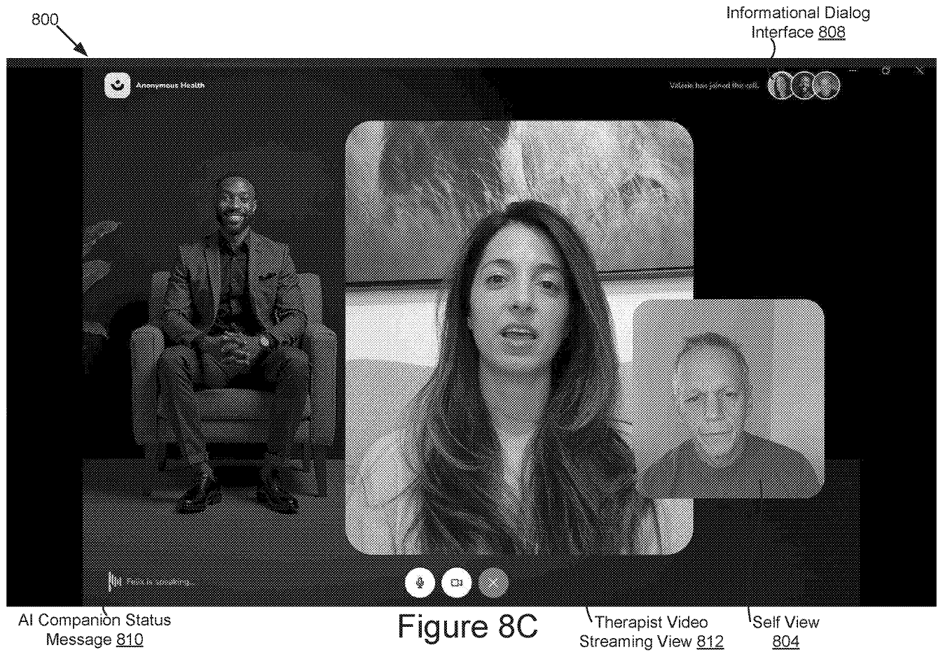

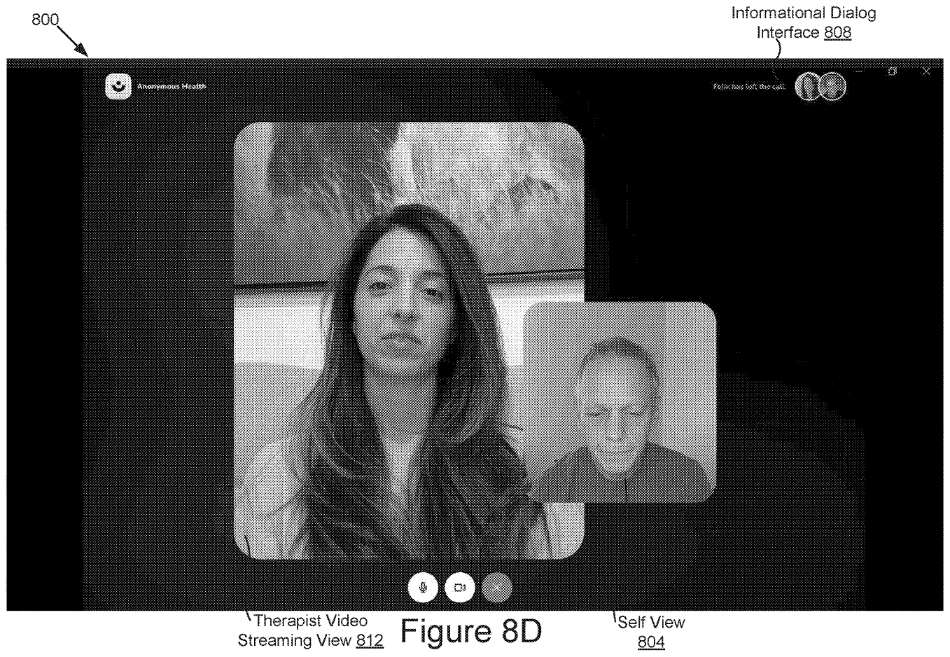

FIGS. 8A-8D are example graphical representations of an example user interface for enabling clinicians and users to interact via an intelligent hybrid therapeutic chat interface. FIG. 8A illustrates an example user interface shown in a screen 800 including an AI Companion, named “Felix” and represented by an avatar on the left hand side of the screen seated on a chair, and a user accessing the therapeutic chat interface for mental health-related issues. Included in the user interface 806 is a series of messages 802 between the AI Companion and the user. In an embodiment, the AI Companion may use text to speech to include an audio message of the same message included in the user interface 800. An informational dialog interface 808 may include information about the clinician, Valerie, joining the call in less than 1 minute. The user interface 806 may further include a self view 804 of the user interacting with the AI Companion. In an embodiment, an AI Companion status message 810 provides information about the AI Companion, such as “Felix is speaking . . . “, indicating that the AI Companion is playing an audio message for the user accessing the therapeutic chat interface. FIG. 8B illustrates screen 800 that depicts an updated user interface 806 that includes a new message by the AI Companion, including a text message that encompasses the AI Companion status message 810.

FIG. 8C depicts screen 800 that now includes a therapist video streaming view 812 as well as an updated informational dialog interface 808 that indicates that” Valerie has joined the call.” Additionally, the AI Companion status message 810 indicates that “Felix is speaking . . . ” to signal the user that an audio message is being played by the AI Companion. For example, the AI Companion may be configured to play an audio message of a summary that has been prepared to inform the human therapist that has recently joined the call about the topics that have been discussed by the user accessing the therapeutic chat interface. FIG. 8D depicts screen 800 that has been updated to remove the avatar of the AI Companion and the informational dialog interface 808 has also been updated to include that “Felix has left the call.”

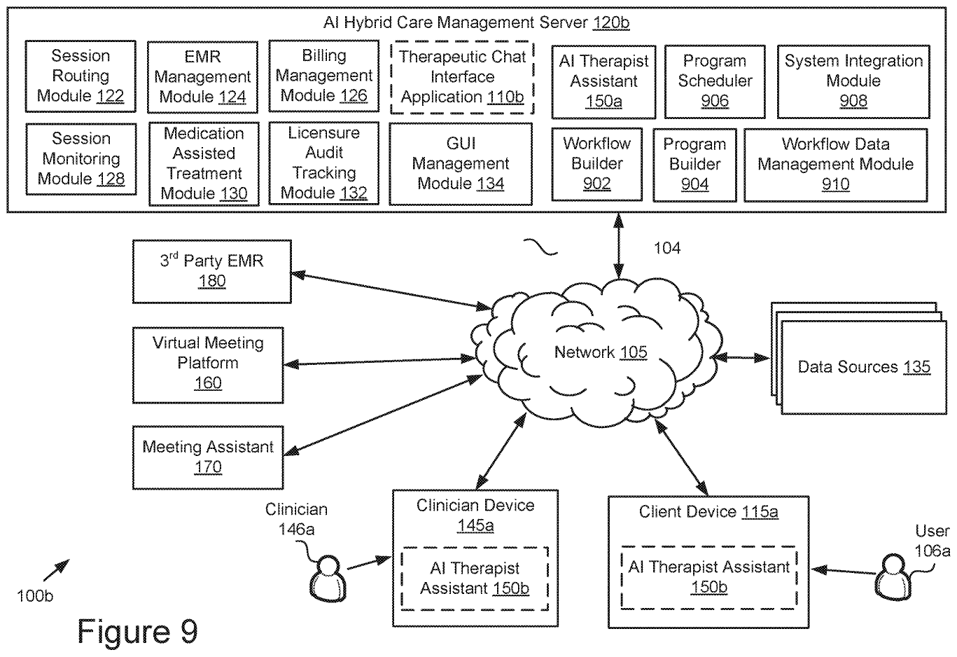

FIG. 9 is a high-level block diagram illustrating a second implementation of an example system for providing users with access to care using an intelligent hybrid therapeutic chat interface using an AI therapist assistant. In this second implementation, the system 100b includes the same components, fewer components, or more components than described above with reference to FIG. 1, but as shown in FIG. 9 has been simplified to show the key elements for an AI therapist assistant 150 with ultra-lightweight EMR integration. This second implementation for the system 100b comprises one or more client devices 115a (even though only one is shown) that can be accessed by users 106, an AI hybrid care management server 120b, a plurality of data sources 135, and one or more clinician devices 145a (even though only one is shown) that are accessed by clinicians 146, all of which are communicatively coupled via a network 105 for interaction and electronic communication with one another. This second implementation for the system 100b also comprises a virtual meeting platform 160 and a meeting assistant 170 coupled to the network 105 for communication with other components of the system 100b.

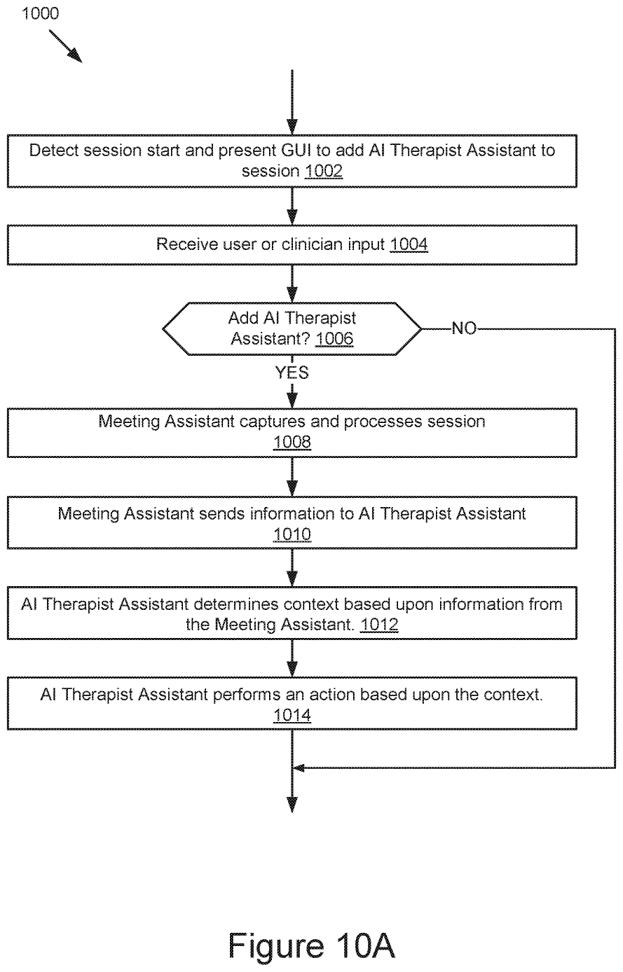

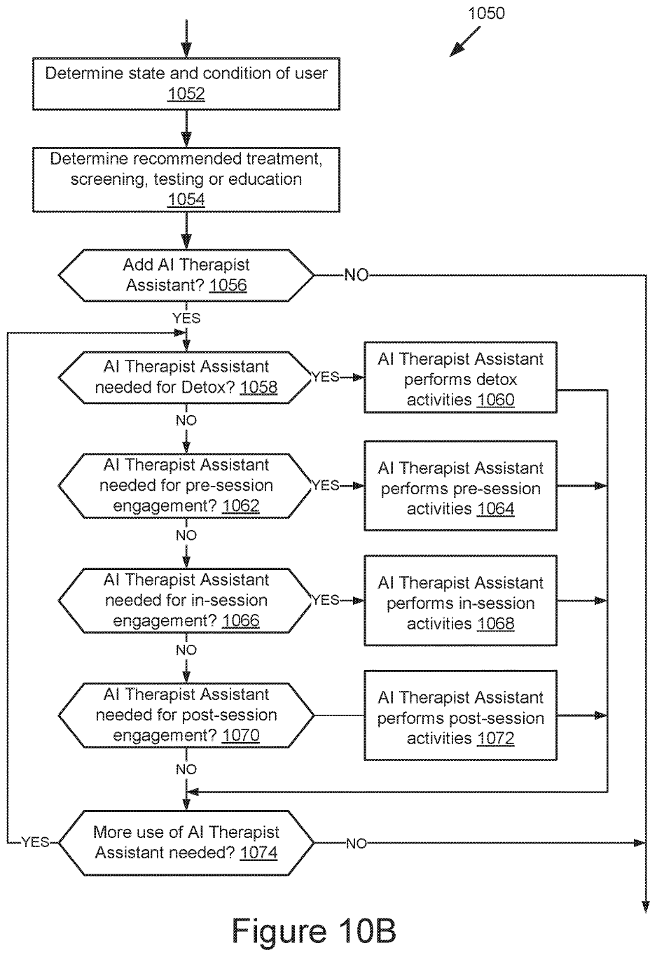

The one or more client devices 115a, the plurality of data sources 135, and one or more clinician devices 145a have the same or similar functionality as has been described above. Differences in the functionality will be described in more detail below in the following paragraphs with reference to FIG. 10. In the system 100b illustrated in FIG. 9 and other figures, a letter after a reference number, e.g., “115a,” represents a reference to the element having that particular reference number. A reference number in the text without a following letter, e.g., “115,” represents a general reference to instances of the element bearing that reference number having a same or similar functionality.

The AI hybrid care management server 120b has the same or similar functionality as has been described above but further comprises the AI therapist assistant 150. In some implementations, the AI hybrid care management server 120b may also include the workflow builder 902, a program builder 904, a program scheduler 906, a system integration module 908, and a workflow data management module 910.

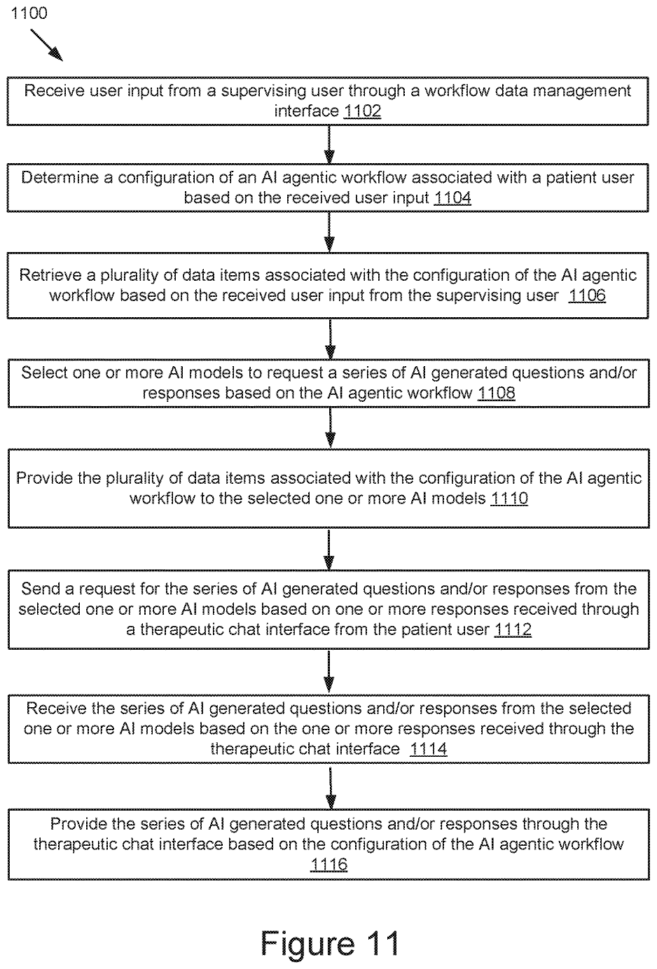

In one implementation, the AI therapist assistant 150a, and its functionality as will be described in more detail below with reference to FIG. 11, is operational on the AI hybrid care management server 120b.

In a second implementation, the AI therapist assistant 150b, and its functionality as will be described in more detail below with reference to FIG. 11, may be operational on the either the clinician device 145a, the client device 115a, or some combination of the clinician device 145a and the client device 115a.

In the third implementation, different components and/or elements of the AI therapist assistant 150 may be operational on the AI hybrid care management server 120b and either the clinician device 145a or the client device 115a. Different configurations of this third implementation may have some components operational on the AI hybrid care management server 120b based upon a number of factors including the computational capability of the AI hybrid care management server 120b, the computational capability of the clinician device 145, the computational capability of the client device 115, available memory or storage at the AI hybrid care management server 120b, the clinician device 145, or the client device 115, and the available bandwidth over the network 105 for communication between these three devices. In one example, where the processing capabilities of the clinician device 145 or the client device 115 are limited, only a few elements of the AI therapist assistant 150b are configured into a thin client that is operated on either of these devices 145, 115, while most of the components are operational on the AI therapist system 150a on the AI hybrid care management server 120b. On the other hand, if the clinician device 145 of the client device 115 is a personal computer with significant processing capabilities and memory, more of the components of the AI therapist assistant 150b may be operational on these devices, particularly when other functionalities such as meetings, AI translation, and form completion are done on the clinician device 145 or the client device 115. Therefore, there may be any number of different configurations which have the different components and/or elements of the AI therapist assistant 150 operational on either the AI hybrid care management server 120b or the clinician device 145a, or the client device 115a.

In a fourth implementation, the AI therapist assistant 150 and its functionality as will be described in more detail below with reference to FIG. 11, may be operational on any third-party server or device, for example, a third-party (3^{rd}) party EMR 180.

Regardless of the implementation, the AI therapist assistant 150 is particularly advantageous because the AI therapist assistant 150 is representation of an actor in the therapy session and the AI therapist assistant 150 joins the session with the therapist and patient and interacts with them. The present disclosure also includes any number of controls of the AI therapist assistant 150 and its operation by the clinician 146 via clinician device 145 or the user 106 via the client device 115. In some implementations, the clinician 146 is blocked from interrupting the AI therapist assistant 150, and the implementation may include web-based controls that the clinician 146 can silently use.

The AI therapist assistant 150 may include software and/or logic to provide functionality for being a digital therapist assistant. The AI therapist assistant 150 performs many of the administrative tasks that would normally be performed by a human therapist. Moreover, the AI therapist assistant 150 can be viewed both by the user 106 and the clinician 146 as an individual that can be added to any therapy session when using the system 100b. This interface by the user 106 and clinician 146 are particularly advantageous because it makes initiation and use of the therapist assistant 150 natural, seamless, and consistent with how an unaided computer therapy sessions would be run. The AI therapist assistant 150 is also coupled for communication and interaction with the virtual meeting platform 160 to control its operation, instruct the virtual meeting platform 160 to perform operations, and send and receive data from the virtual meeting platform 160. Similarly, the AI therapist assistant 150 is also coupled to the meeting assistant 170 for communication, interaction and control of the meeting assistant 170 to perform operations and send and receive data from the meeting assistant 170. The coupling of the AI therapist assistant 150 to the virtual meeting platform 160 and the meeting assistant 170 allows seamless integration between the three components that allows the AI therapist assistant 150 to be added easily and seamlessly to any meeting.

The AI therapist assistant 150 is coupled to the other components of the AI hybrid care management server 120b for communication, interaction, and cooperation with them. For example, the AI therapist assistant 150 may interface with any of these components to perform their respective function. For example, the AI therapist assistant 150 is coupled for interaction with the EMR manager module 124. This allows the AI therapist assistant 150 to initiate any documentation needed or required based on a therapy session. The information from their position is automatically captured, recorded, transcribed and provided by either the virtual meeting platform 160 or the meeting assistant 170 to the AI therapist assistant 150. The AI therapist assistant 150 can then cooperate with the EMR manager module 124 to store that information where required, complete forms as necessary and perform any other documentation activity. Similarly, the AI therapist assistant 150 is coupled for communication with the therapeutic chat interface application 110, in particular, the therapeutic chat interface engine 206. By cooperating with the therapeutic chat interface engine 206, the AI therapist assistant 150 is able to send and receive information to it and thereby determine the context or any therapy session or portion of the session. This determined context can then be used by the AI therapist assistant 150 to initiate or trigger other actions based on that context. The AI therapist assistant 150 can initiate actions by any module of the AI hybrid care management server 120b based on that context. In yet another example, the AI therapist assistant 150 interacts with the artificial intelligence platform 325 (see FIG. 3) of the AI hybrid care management server 120b to perform any variety of AI functions and create specialized bots 335 on the artificial intelligence platform 325 to complete the desired function.

One example implementation of the operation of the AI therapist assistant 150 is described in detail below. It should be noted that other implementations of the AI therapist assistant 150 may include any subset of the steps of operation described below. Moreover, AI therapist assistant 150 may perform operations in addition to those described below. Additionally, in some cases, the operations may be performed in an order different than described herein. In an example implementation of the operation of the AI therapist assistant 150, the AI therapist assistant 150 first joins a 3^{rd} party Virtual Meeting Platform 160. In one implementation, the AI therapist assistant 150 joins a 3^{rd} party Virtual Meeting Platform 160 that is preferably integrated to a 3^{rd} party EMR 180. Then, the AI Therapist Assistant obtains the patient record and other information from the 3^{rd} party EMR 180 to provide context for the AI Therapist Assistant 150. For example, the patient record may be retrieved through an application programming interface (API) or through the 3^{rd} party EMR 180 through a web tool, such as a document object model (DOM). The AI Therapist Assistant 150 retrieves contextual data from the 3^{rd} party EMR 180 and/or the data storage elements of the AI Hybrid Care Management Server 120b, and preferably combines this data with the context and data from the current session interactions, to inform an AI prompt and/or to direct the AI Therapist Assistant 150 on a progression across a defined operational and/or clinical workflow via the Session/Workflow Orchestrator 1408. Contextual data may have been previously defined by an administrative user through a workflow builder prompt, in one implementation.

In an implementation, the AI Therapist Assistant 150 identifies recommendations for the therapist and presents these to the therapist within the session via the GUI Management Module 134, and/or generates Tasks or other follow-up in the 3rd Party EMR 180 and/or other systems used by the parties which may include but are not limited to the therapist, the therapists' supervisors, other members of the patients' clinical care team, operational staff, an insurance company, or a managed services organization (MSO). For example, the AI Therapist Assistant 150 identifies indicators of a potential medical emergency or crisis and activates medical or crisis escalation protocols including potentially activating emergency services such as a 911 or 988 hotline. In an implementation, the AI Therapist Assistant 150 processes the entirety of the session between the therapist, the patient, and the AI Therapist Assistant 150, and uses this data as context for one or more prompts in order to populate one or more forms or clinical documentation. These forms may be stored in the AI Hybrid Care Management Server 120b and/or the 3rd Party EMR 180.

The AI Therapist Assistant assesses eligibility for an insurance claim vis-à-vis criteria stored in the Billing Management Module 126 or in the Billing Management Module of the 3^{rd} party EMR 180 based on data collected from the session which may include but is not limited to time in session; assessments of ICD-10 or other diagnoses codes; assessments of complexity of the patient's condition; the division of time of interactions between the therapist and the patient, and the Therapist Assistant and the patient; or other criteria that may inform billing amounts and/or eligibility.

The AI Therapist Assistant 150 receives an input via the Therapeutic Chat Interface Application 110b from the therapist to redirect the interaction with the patient, which may include but is not limited to: (i) selecting a different workflow or content for the AI Therapist Assistant 150 to focus on with the patient, (ii) to pause or resume engaging with the patient, (iii) to receive additional context from the therapist, (iv) to correct the interactions of the AI Therapist Assistant 150, (v) to capture or record information to a form, (vi) to annotate a portion of the transcript or analysis of the interaction as an attachment to a portion of the medical record such as diagnosis code, (vii) to create a follow-up Task which may include as an attachment or as context a transcription or analysis based on all or part of the interaction, or (viii) evaluation of the interaction of the AI Therapist Assistant 150 for use in reinforcement learning or model training.

The AI therapist assistant 150 is particularly advantageous because it provides ultra-lightweight EMR integration. In one implementation, the AI therapist assistant 150 is required to be integrated into the EMR system to a) perform the read/write EMR operations and b) perform the voice/text operations for the session from the AI Therapist Assistant 150. In some implementations, the AI therapist assistant 150 interacts with the patient and/or therapist by voice within the session itself, and initiates AI agent(s) to scribe data to and otherwise interact with the EMR manager module 124 to ultimately store data in third-party EMR systems. For example, the AI Therapist Assistant 150 interacting with the patient or therapist by voice within the session itself, the best lightweight approach is for the AI Therapist Assistant 150 to be an invitee to the scheduled telehealth appointment (similar to how notetaker apps like ReadAI operate as invitees to Google Meet and other tele meeting platforms). In another approach, in situations in which the bandwidth of the patient or therapist is limited, the patient, therapist, or AI Therapist Assistant 150 may access the session by dialing in via traditional telephone lines rather than VoIP, and thus leverage longstanding infrastructure including audio codecs, packet handling and network protocols, session initiation protocol (SIP), and other infrastructure to support call quality. The AI Therapist Assistant 150 is also able to interact with the 3^{rd} party EMR systems, for instance, to scribe data to forms or take other actions, it is insufficient to be an invitee to the meeting. For this reason, the AI Therapist Assistant 150 also preferably leverages a browser extension. The browser extension performs the functions of scribing information to the 3^{rd} party EMR system and doing other interactions with the EMR system.

The virtual meeting platform 160 is an online video conferencing platforms accessible to the AI therapist assistant 150 over the network 105. The virtual meeting platform 160 may include various functionality including but not limited to high-quality video and audio conferencing that enables real-time face-to-face meetings, often with HD video and noise suppression for clarity; screen sharing that allows presenters or participants to share their entire screen, a specific application, or document to the group, enhancing presentations and discussions; chat and messaging that allows chat features let users send messages, links, or files privately or to the whole group, without disrupting the meeting flow; recording and playback that allow meetings to be recorded and saved for later reference or for those who could not attend live; breakout rooms for larger meetings or webinars can be split into smaller groups for focused discussions or activities, then reconvened to the main session; whiteboard and annotation tools that enable visual collaboration, brainstorming, or annotation of documents during the meeting; file sharing and integration that provide the ability to share files directly within the platform;

transcription and meeting summaries that may use AI to generate live transcripts and meeting summaries for accessibility and documentation; and other accessibility features such as pinning sign language interpreters, enlarging videos, and supporting relay services for participants with disabilities. For example, the virtual meeting platform 160 may be any platform like Google Meet, Zoom, Microsoft Teams, Cisco Webex, Apple Facetime or similar services.

The meeting assistant 170 is an artificial intelligence platform designed to automate the process of capturing, transcribing, summarizing, and analyzing meetings, conversations, and documents across digital and in-person settings. The meeting assistant 170 may include various functionality including but not limited to automatically producing detailed notes, highlights key moments, and generates concise overviews of meetings based on content, engagement, and sentiment; live transcription that provides real-time, highly accurate transcriptions for both virtual and in-person meetings, with unique speaker detection to distinguish between contributors; engagement and analytics that measure engagement, talk time, and provides coaching tips (such as recommendations for pacing and fewer filler words) to help improve meeting effectiveness; cross-platform integration that connects with various platforms, including email, chat, CRM, and workflow tools, enabling unified search and access to insights across all organizational content; and enterprise search that unifies and enables rapid search of insights from meetings, messages, emails, documents, and more-delivering instant, AI-powered answers to user queries, directly within existing workflows; multi-language support that handles over 20 languages and continually adds more for broader accessibility; Optical Character Recognition (OCR) that converts scanned images or photographs of text into editable, searchable formats for easier document management. In some implementations, the meeting assistant 170 may be incorporated or be a part of the virtual meeting platform 160. Examples of the meeting assistant 170 include Read.ai, Fireflies.ai, otter.ai, tl;dv, Avoma, plus many others.

The operation of the workflow builder 902 and its cooperation with the other components of the AI hybrid care management server 120 will be described in more detail below and is particularly advantageous because it allows clinicians and others without technical backgrounds or programming experience to create AI Therapist Assistant content and related workflows including creation of a workflows or a form as discrete, configurable pieces of clinical content. The workflow builder 902 may include software and/or logic to provide functionality for providing a critical part of the backend of the AI hybrid care management server 120 because it allows the clinicians and administrators to customize and optimize the operation of the AI assisted mental health counseling to meet a myriad of different requirements. Using the workflow builder 902, a clinician can create the building blocks for mental health counseling, leveraged those building blocks into workflows, specify how context and other information is passed between nodes in the workflow, define how a workflow will be triggered or conditions that will initiate or trigger a workflow, and define the relationship between AI, human tasks, and custom forms. The workflow builder 902 provides the infrastructure that enables the program builder 904 and the program scheduler 906 to operate to create user-configurable mental health programs from one or more configurable program content blocks, as described herein.

In some implementations, the workflow builder 902 is a visual, node-based editor that enables construction of healthcare workflows from discrete building blocks. For example, the discrete building blocks or nodes include prompt nodes, context nodes, form nodes, condition/event nodes, variable nodes, and interaction nodes. A prompt node defines AI instructions and prompt configuration. A context node manages variables and contextual data across the workflow. The form node generates dynamic forms for patient or clinician input. The condition/event node handles branching logic, trigger transitions, and/or events. The variable node centralizes global state and provides variables that can be used globally across two or more workflows or locally only in one workflow. The interaction node is a node for reading or writing to a 3^{rd} party system. The workflow builder 902 with these nodes advantageously provides a low-code/no-code structure that enables non-technical users to compose complex workflows that blend AI and human tasks. A key element is the inclusion of executable if-then logic that provides explainability for AI-driven decisions. This is particularly important in regulated industries such as healthcare, where auditability and interpretability of LLM actions are critical. The workflow builder 902 is an extensible system for building AI-driven and human-in-the-loop clinical workflows. By treating forms, prompts, and context as composable nodes, and enabling real-time orchestration and collaboration, the platform enables creation of configurable “clinical content workflows” that go beyond static pathways. With event nodes for external triggers and if-then explainability logic, the workflow builder 902 provides both technical power and the transparency necessary for regulated healthcare use cases.

Program builder 904 may include software and/or logic to provide functionality for creating and configuring mental health programs to be delivered by an AI therapist assistant 150a and/or through a therapeutic chat interface application 110b. For example, a program builder 904 may use a workflow builder 902 as described above to enable a user of the AI hybrid care management server 120b to configure AI engagements with patients through a series of user interfaces that enable the creation and configuration of one or more program content blocks that make up a mental health program.

Program scheduler 906 may include software and/or logic to provide functionality for generating a schedule for mental health programs that are delivered through an AI therapist assistant 150a and/or through a therapeutic chat interface application 110b. For example, a program scheduler 906 may use a workflow builder 902 as described above to enable a user of the AI hybrid care management server 120b to retrieve an availability of one or more clinician users for an AI hybrid session with an AI therapist assistant 150a. In some implementations, a recurring appointment is scheduled based on an availability of a selected clinician as well as the preference of the patient user. In other implementations, a program may be scheduled to be triggered from an event happening, such as an admission date into a residential treatment center, a completion of another program delivered through the AI therapist assistant 150a and/or through the therapeutic chat interface application 110b, or a behavior or set of user interactions that have been identified by the AI therapist as indicating that the patient is in crisis or at risk of engaging in unwanted behavior, for example. These custom trigger events may also be created and configured through the program scheduler 906, in various implementations.

System integration module 908 may include software and/or logic to provide functionality for integrating various functionality between the AI hybrid care management server 120b and third-party systems accessible through network 105. For example, a third party system, such as a customer relationship management (CRM) system, may have functionality such as Tasks that are accessible by an application programming interface (API) through network 105 that enable a user to create and modify the functionality, such as creating a task to follow up with a patient to schedule an in-person medical appointment, for example. The system integration module 908 enables the workflow builder 902 to access APIs of third-party systems to enable functions to be triggered based on events that occur within the AI hybrid care management server 120b and/or system 100b.

Workflow data management module 910 may include software and/or logic to provide functionality for enabling a supervising user to configure AI companion(s) through a workflow-based graphical user interface. For example, the workflow data management module 138 may include various modules that enable the supervising user to gather and/or enable additional collection of user information, provide a form to a patient user to execute a questionnaire and/or protocol for various health treatment, enable the supervising user to configure the generative AI model to execute a set of explicit instructions, interpret a set of instructions to be transformed into a conversational style for the AI companion, invoke and/or activate one or more software applications and/or software functionality to provide additional information and/or resources for the patient user, and provide a customized workflow for additional prompts, context, instructions, and/or applications to be provided to the patient user through the AI companion and AI session(s).

FIG. 10A is an example flowchart diagram of a method of operation of an AI therapist assistant automatically scheduling and conducting mental health exercises for a user in accordance with the present disclosure. Referring now to FIG. 10A, one implementation for a method 1000 executed by the AI therapist the system 150 will be described. The method 1000 begins by monitoring for the initiation of a therapy session on a virtual meeting platform 160. Next, the method 1000 detects 1002 the start of the therapy session and presents a user interface to add the AI therapist assistant 150 to the session. In one example, a user interface with a selectable button is presented on the meeting GUI by the virtual meeting platform 160. The button is selectable by either the user 106 or the clinician 146. In another example, an audio message is sent by the AI therapist assistant 150 to the virtual meeting platform 160 to be played in the session with the question of whether either party wants to add the AI therapist assistant 150 to the virtual meeting. The audio message is played by the virtual meeting platform 160 and the virtual meeting platform 160 is instructed to send back to the AI therapist assistant 150 any response from either the user 106 or the clinician 146. The method 1000 continues by receiving 1004 a response/input from either the user or clinician. The method 1000 continues to determine 1006 whether the AI therapist assistant should be added to the virtual meeting. If not, the method 1000 is complete and ends. On the other hand, if either party has requested that the AI therapist assistant be added to the session, the method 1000 proceeds to block 1008. In block 1008, the meeting assistant 170 is instructed to capture and process all communications during the virtual meeting. Next, the meeting assistant 170 sends 1010 the information to the AI therapist assistant 150. The method 1000 continues with the AI therapist assistant 150 determining context 1012 based upon information from the meeting assistant 170 and/or the EMR management module 124 and/or the medication assisted treatment module 130, and/or other sources of context that may be available. Then the AI therapist assistant 150 analyzes the context and information from the meeting assistant 170 and/or other sources and performs 1014 an action based upon the context. For example, AI therapist assistant 150 may determine that information needs to be stored in the user's EHR. In such a case, the AI therapist assistant 150 cooperates with the EHR management module 124 to store the information in the digital file of the user. In another example, the AI therapist assistant 150 determines that the action to be taken is to communicate with the workflow engine and mark a step as completed, modify the workflow process, initiate additional workflows, or take any other workflow action. For example, the AI therapist assistant 150 includes the session/workflow orchestrator to connect, instruct and communicate with a workflow execution engine 1506 and a workflow builder 902. In such a case, the AI therapist assistant 150 determines the workflow action to be taken and cooperates with the workflow execution engine 1506 to complete that action. In yet another example, the AI therapist assistant 150 determines that a form needs to be completed. Based on the information from the virtual meeting platform 160 and the Session/Workflow Orchestrator 1408, the AI therapist assistant 150 determines not only the form to be completed but also populates the form with the information from the virtual meeting. The above are just a few examples of what can be performed with the AI therapist assistant 150.

The understanding of the operation and interaction of the AI therapist assistant 150 can be better understood by walking through an example of the actions performed by the AI therapist assistant 150 during an online therapy session in a virtual meeting. The side steps and sub steps of such an example therapy session are described below. Therapist Valerie (i) adds an AI Therapist Assistant 150 to their existing scheduled meetings in their existing EMR to support the interaction of the AI Therapist Assistant 150 with the patient and the therapist (similar to how a notetaking app works such as Read.ai), and adds a browser extension that enables the AI Therapist Assistant 150 to read and write to the existing EMR (similar to how a scribe app works, such as Eleos). The AI Therapist assistant 150 can engage the patient directly in a voice dialogue, providing additional support to the patient and much-needed respite to the therapist, who is actively listening.

Therapist Valerie starts the meeting with Sarah, the Patient. The AI Therapist Assistant 150 can be given a pseudonym, e.g., Calvin, and automatically joins the meeting at the start of the meeting. Calvin is an AI Agent that automatically takes records of the audio and takes notes which can be written to an EMR. Calvin and other AI agents are listening to the call and may have other criteria based on the discussion that may trigger one or more events which in turn activate system workflows. In the preferred instantiation, the AI Agent is trained or prompted to listen for its name during the discussion between Valerie, the therapist, and Sarah, the patient.

Therapist Valerie starts the session, and says, “Sarah, I'd like to begin by doing an ABC Thought Record exercise. Recall that we went over how this works in our last session. Is that okay with you?” Upon agreement by Sarah, Valerie vocally activates Calvin, the AI Therapist Assistant, to lead Sarah through the ABC Thought Record exercise. e.g., “Calvin, can you please assist me and Sarah with an ABC Thought Record exercise? When finished, can you please work with Sarah to complete the GAD-7 anxiety screener?” Calvin is prompted to listen for an activation of this type and thereby engages vocally with Sarah during the session. Calvin follows the instructions it has been given by Sarah, and this is achieved by executing two workflows in serial order that specify Calvin's engagement with the patient—in this case: (1) the ABC Thought Record workflow and (2) the GAD-7 anxiety screener workflow.

Calvin runs through the content of the two workflows with Sarah. One or more AI Agents are listening to this part of the session. Based on the dialogue, the AI Agents may be triggered to generate events which in turn may activate one or more system actions or workflows. By way of example, the system actions or workflows might include: (1) transcription, (2) creating clinical documentation, (3) triggering a crisis or escalation workflow, (4) triggering a Task to be assigned to one or more persons, roles or groups, (5) scheduling follow-up engagements, (6) triggering a workflow instance of any number of defined workflow templates. The AI Therapist assistant 150, Calvin, transitions back to the client at the completion of the workflows that it had been instructed to complete, plus other workflows that may have been activated in the course of the engagement with Sarah, the patient. The notes from the whole session are summarized by an AI Agent to the EMR, and forms and other documents may be created and stored. Multiplied by 5+appointments per day, the therapist gets a massive, game changing respite every single day and can be more focused during the session by not having to take notes.

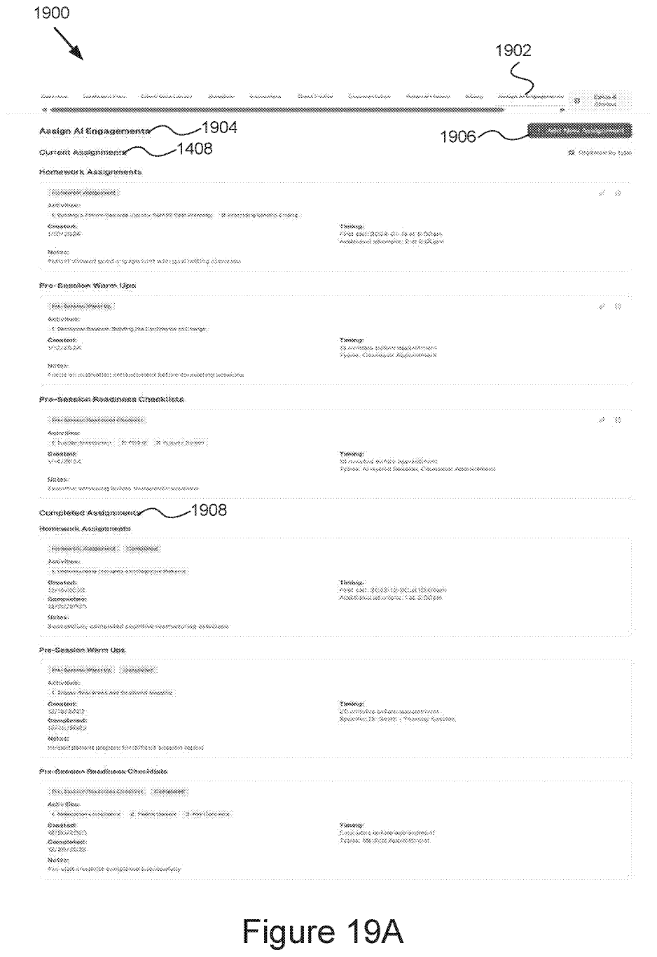

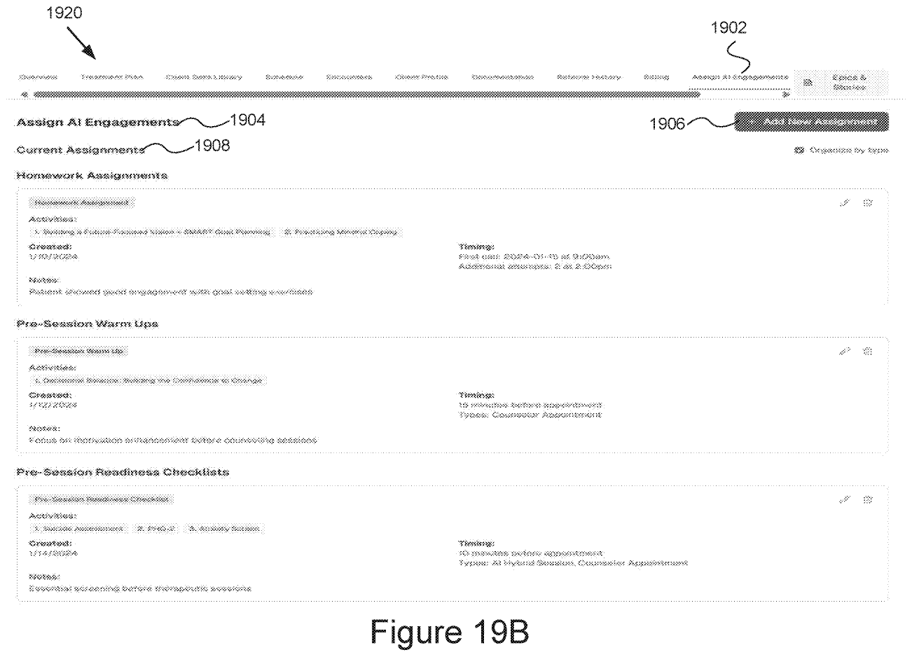

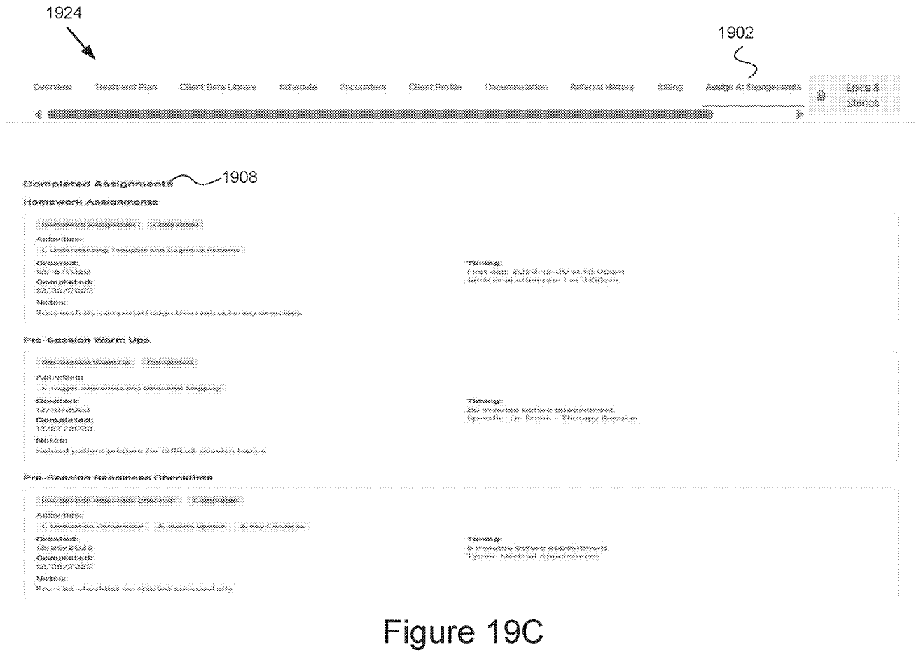

FIG. 10B is an example flowchart diagram of a method of operation of an AI therapist assistant automatically scheduling and conducting mental health exercises for a user in accordance with the present disclosure. Referring now to FIG. 10B, a method 1050 for automatically scheduling and conducting mental health exercises for a user by an AI therapist assistant 150 is described. The method 1050 begins by determining 1052 a state and/or condition of the user 106. As has been described above, this determination may be made automatically by the AI hybrid care management server 120 and may access a variety of resources about the condition of the user 106 including medical records, medications prescribed, therapist records, counseling records, program information, and a variety of other sources that are indicative of the mental state of the user 106. In some implementations, the AI therapist assistant 150 determines the state and/or condition of the user 106 as has been described above. The method 1050 continues by determining 1054 a recommended treatment, screen, testing, or education to be performed based upon the state and/or condition of the user determined in block 1052. The determination in block 1054 may be as simple as determining when the next therapist session is going to occur and identifying one or more tasks that the therapist has specified that the user 106 should perform. One example interface for assigning tasks for the AI therapist assistant 150 to perform and monitoring completion of the assigned tasks will be described in more detail below with reference to FIGS. 19A-19C. In more complex situations, the AI hybrid care management server 120 may perform complex analysis of any number of factors and information described above and prescribed a recommended treatment, screen testing or education that the user should be required to perform. In some implementations, the AI hybrid care management server 120 makes a recommendation which must be approved by the therapist of the user 106 before it is added as a required task that the user must perform. The method 1050 continues by determining 1056 whether the AI therapist assistant 150 should be used to perform any portions of the treatment determined in block 1054. In some implementations, both the user 106 and the therapist must agree to using the AI therapist assistant 150 to perform the portions the mental health interaction and treatment in lieu of the therapist or other human counselor. If it is determined that the AI therapist assistant 150 cannot be used, the method 1050 is complete.

On the other hand, if it is determined that the AI therapist assistant 150 can perform any number of tasks that represent a portion of the therapist's activities, such as through a workflow configured using a workflow builder as described herein, the method 1050 proceeds to block 1058. In block 1058, the method 1050 determines whether the AI therapist assistant 150 should be used to engage the patient with one or more therapeutic exercises or conversational questionnaires to support the clinical engagement. If not, the method 1050 proceeds to block 1062 and tests for other conditions as will be described below. However, if the method 1050 determines that the AI therapist assistant 150 should be used to perform one or more activities related to the user 106 that support the clinical engagement, the method 1050 proceeds to block 1060. In block 1060, the AI therapist assistant performs one or more activities to support clinical engagement. For example, the AI therapist assistant 150 may interact with users that are in detox to keep them engaged and reduce the likelihood that they will leave detox. In this block, the AI therapist assistant 150 is used to fight boredom and drive clinical progress. Such activities that the AI therapist assistant 150 may perform include, but are not limited to, playing interactive games with the user 106, sending text messages to the user 106, showing videos or other content that will engage the user 106 and distract the user from the pain of the intoxication, activities to prevent boredom, activities to support recovery, activities to distract from the pain of detox, motivational interviewing, presentation of decision balance content, journaling exercises, guided meditations, breathing exercises, Q&A about books the user is reading, poetry cultivation, beat and loop tools for musical users any variety of Cognitive Behavioral Therapy (CBT) exercises, or any other activity that will reduce the likelihood that a user 106 will leave a supervised residential rehabilitation stay or be discharged against medical advice (“AMA”). In some implementations, information about the withdrawal management (i.e. “detox”) activities that are being performed by the AI therapist assistant 150 may be sent to others in addition to the clinician 146. The clinician 146 may use the assignment, tracking, and completion of activities for safety assessment while in detox. However, during any detox session (or other supervised residential rehabilitation stay), the assignment, tracking, and completion tasks by the user 106 may also be to medical personnel for evaluation or specific handling of a medical condition. For example, if the patient/user 106 is in withdrawal they may need to seek medical immediately and other AI agents or bots can send messages to medical personnel or a primary care provider or call 911 depending on the medical condition, e.g., confusion, hallucinations, seizures, and autonomic hyperactivity occurring in the detox process.

In some implementations, if the user 106 is not responsive to queries or interactions by the AI therapist assistant 150 while in detox, the AI therapist assistant 150 and automatically perform follow-up interactions, send a message to counselor, the facility, or their therapist based on any number of predefined protocols, analysis of user condition, or other factors. In some implementations, a user 106 in detox may be assigned a given workflow with many different steps where content or pieces are assigned incrementally over time. For such implementations, the AI therapist assistant 150 monitors the workflow and the progression of the user along the workflow, and the AI therapist assistant 150 can unlock different content, features, or functionality based upon the position in the workflow of the user. More specifically, the detox workflow may have an initial phase of the first 24 hours where new content is released every two hours, and the second phase after the first day where additional activities and content are unlocked every four hours or some other time different than the first phase. This prevents the user in detox from engaging with the AI therapist assistant 150 nonstop in completing the entire workflow in less than an optimum amount of time that allows the user 106 to complete detox. After block 1060, the method 1050 proceeds to block 1074 as will be described below.

In block 1062, the method 1050 determines whether the AI therapist assistant 150 should perform pre-session engagement. If not, the method 1050 proceeds to block 1066 and tests for other conditions as will be described below. On the other hand, if the method 1050 determines that the AI therapist assistant 150 should perform pre-session engagement activities with the user 106, the method 1050 proceeds to block 1064. In block 1064, the AI therapist assistant 150 performs pre-session activities. For example, pre-session activities include, but are not limited to, pre-session questionnaires, screening questions, the session checklist, running through questionnaires with the user 106 about their condition, anxiety, depression, medication usage, etc., performing warm up interaction with the user 106. Specific examples and timing for questions by the AI therapist assistant 150 are listed herein. First, on a pre-session day before, client/patient is given a screener of questions, such as Patient Health Questionnaire-2 (PHQ-2) that asks about the frequency of depressed mood and anhedonia over the past two weeks, if they feel like harming themselves, or if they are anxious with a Generalized Anxiety Disorder 2-item (GAD-2) initial screening tool. For example, two questions may be asked, such as “Over the last 2 weeks, how often have you been bothered by the following problems? A) Feeling nervous, anxious, or on edge B) Not being able to stop or control worrying” with options for response as “Not at all” (0 points), “Several days” (1 point), “More than half the days” (2 points) and “Nearly every day” (3 points). The GAD-2 score is obtained by adding score for each question (total points). An interpretation of a score of 3 points is the preferred cut-off for identifying possible cases and in which further diagnostic evaluation for generalized anxiety disorder is warranted. Using a cut-off of 3, the GAD-2 has a sensitivity of 86% and specificity of 83% for diagnosis of generalized anxiety disorder. Other types of screening questionnaires and/or tools to help diagnose various mental health issues may be implemented in a workflow to be executed by the AI therapist assistant 150.

Another example and timing for questions by the AI therapist assistant 150 includes performing a check of lists of substances/behaviors used including vape/tobacco, if they are taking medicines as prescribed, if they have fallen, and any areas of concern they want to talk about today this could be from drop down menu or free text. Yet another example includes, on the pre session day of, providing a user interface where the patient sees a menu of CBT exercises they have completed (locked), others that they have yet to complete (unlocked), asterisk next to the one or two chosen by therapist week before to do, they can open one of their choosing. Once finished the AI therapist assistant 150 thanks them for their work and states their therapist 146 will be with them shortly. A further example illustrates options on the therapist side. For example, the clinician 146 will get an alert as soon as the pre session day before is done and see results. Finally, on the day of the session, the clinician 146 will have an opportunity to read the script that the patient/user 106 just finished with the AI therapist assistant 150 and be able to focus attention on what was just mentioned.

More particularly, the pre-session engagements that the AI therapist assistant 150 may perform include a T−24 hours checklist for suicidality, PHQ-2, anxiety screen, meds refill check, substances used, key concerns, etc., or, at T−15 minutes: warm-pp exercises for CBT exercises they haven't done before, identification of areas of concern, and notification that clinician will be in shortly. In some implementations, where the user 106 is not responsive to queries or interactions by the AI therapist assistant 150, the AI therapist assistant 150 automatically perform follow-up request, for example, 4 days before the session, 3 days before the session, 2 days before the session, 1 day before the session, 12 hours before the session, etc. In some implementations, the time when the AI therapist assistant 150 should initiate recession activities is predefined or set by the therapist. For example, for a warm-up session, the therapist may prescribe that that warm up session began 15 minutes or some other predefined time before the session with the therapist begins. Also, at any point in time, the AI therapist assistant 150 may send messages to others indicating that the user 106 is not responsive and may even reschedule the session if the user is not responsive. After block 1064, the method 1050 proceeds to block 1074 as will be described below.

One particular pre-session engagement that the AI therapist assistant 150 may perform is a warmup visit. In many instances, a significant part of a therapist session with a patient is the warm-up portion wherein the user is flustered and don't immediately open up. The AI therapist assistant 150 can warm-up the patient with CBT exercises such as in this example:
Monday evening-prior to appointment
   AI companion connects with a patient via telephone or app.
   AI companion completes a pre-visit checklist (every session).
   Checklist items: suicidality, PHQ-2, anxiety screen, meds good and need for refill, tobacco, fallen, substance used, behavior engaged, key concerns
Tuesday-day of appointment
   11:00-11:10-Patient warm up with AI companion.
      Pick CBT exercise that patient hasn't done yet
      AI asks about any areas of concern
      AI notifies a patient that therapist will be with you shortly
   11:00-11:10-Therapist notified of key issues
      AI notifies if patient didn't do their CBT exercise.
      AI highlights key issues.
      (Younger generation loves this type of engagement model)
   11:10-11:45-Therapist session and the patient has been warmed up and is willing to share concerns with Therapist.
   11:45-11:50-Therapist assigns CBT homework exercises from library
      Pick two to work on this week.
      Clinicians use expertise to select type and timing of CBT exercise (therapist can control the lock and unlock CBT exercises based on their expertise and interaction.)

In block 1066, the method 1050 determines whether the AI therapist assistant 150 should perform in-session engagement activities. If not, the method 1050 proceeds to block 1070 and tests for another condition as will be described below. On the other hand, if the method 1050 determines that the AI therapist assistant 150 should perform in-session engagement activities with the user 106, the method 1050 proceeds to block 1068. In block 1068, the AI therapist assistant 150 performs the in-session activities. Example in-session activities that may be performed by the AI therapist assistant 150 have been described above so that description will not be repeated here. At any point in time, the AI therapist assistant 150 may send messages to the therapist or others indicating that the user 106 is not responsive. After block 1068, the method 1050 proceeds to block 1074 as will be described below.

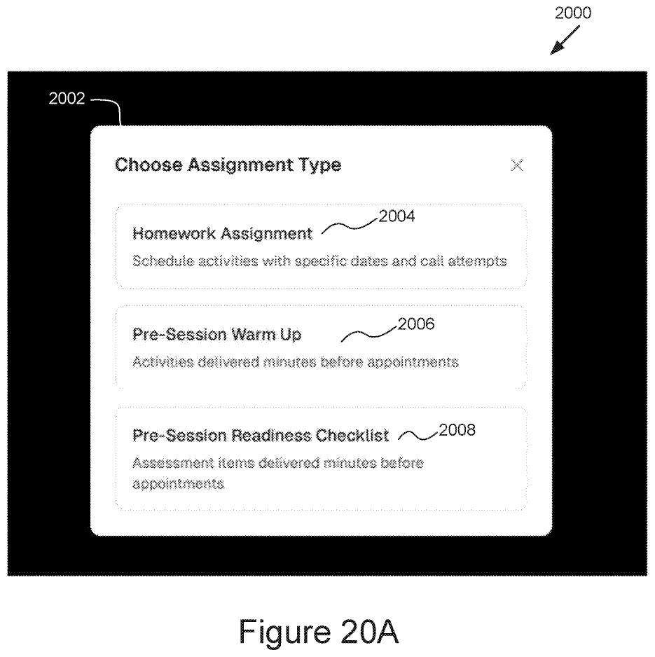

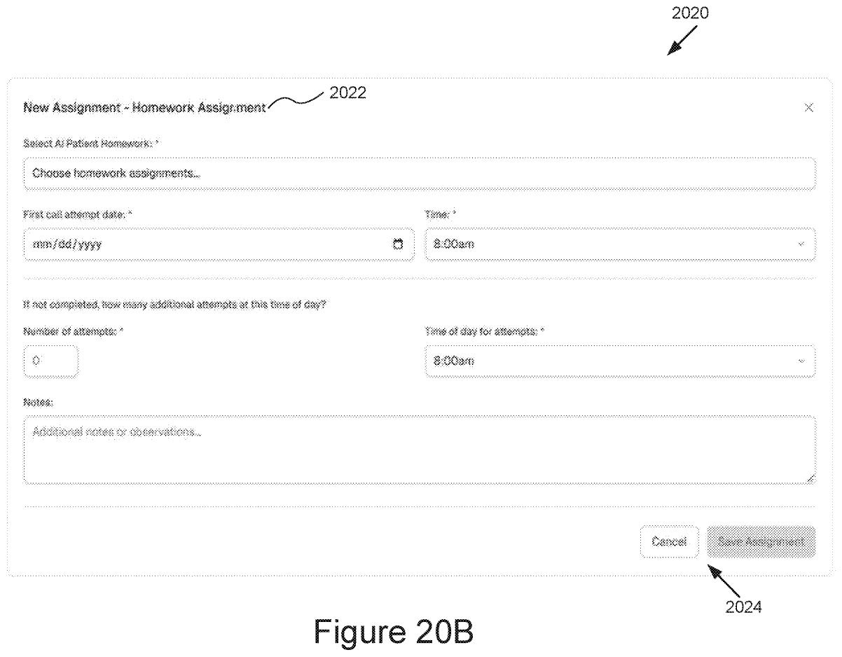

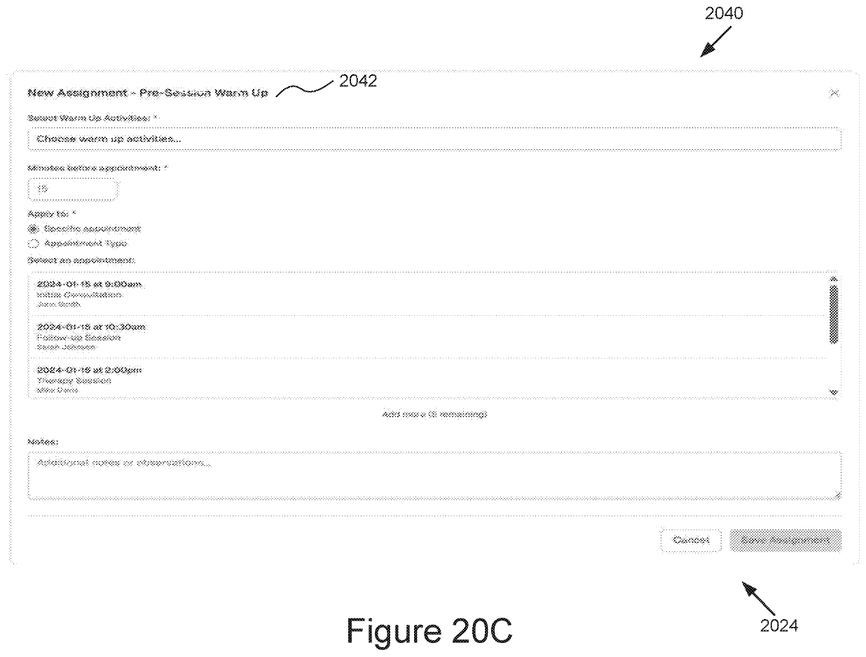

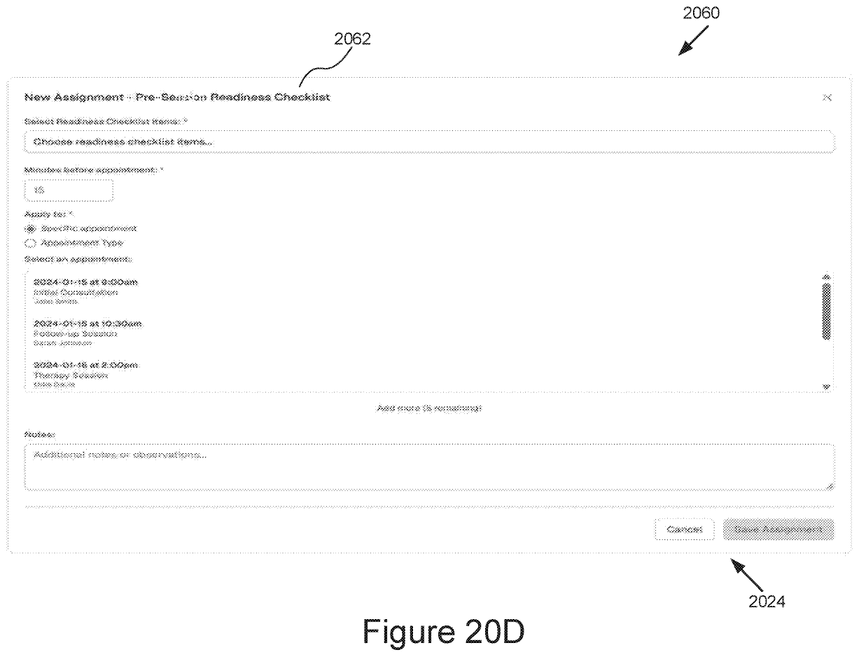

In block 1070, the method 1050 determines whether the AI therapist assistant 150 should perform post-session engagement activities. If not, the method 1050 proceeds to block 1074 as will be described below. On the other hand, if the method 1050 determines that the AI therapist assistant 150 should perform post-session engagement activities with the user 106, the method 1050 proceeds to block 1072. In block 1072, the AI therapist assistant 150 performs the post-session activities. Example post-session activities include, but are not limited to, assigning homework, performing post-session exercises with the user, follow up on the completion of homework, monitoring and repeated prompting if homework is not complete, follow-up connections with the user at specified times or intervals. In some implementations, near the end of a session, as the therapist/clinician 146 is wrapping up, they can choose another CBT exercise, repeat one that was done, ask if the client wishes to have other exercises re-opened, ask how patient/user 106 felt about their AI session, ask what alerts does the patient/user 106 wants as reminders from the AI to support positive changes, and as if there needs to be linkage to medical provider to do so promptly. The therapist/clinician 146 can also use the GUI described below with reference to FIGS. 19A-19C and 20A-20D to create new assignments for the AI therapist assistant 150 to perform based on this end of session discussion with the patient/user 106.

At any point in time, the AI therapist assistant 150 may send messages to the user 106, their therapist, or others indicating that the user 106 is not responsive. After block 1072, the method 1050 proceeds to block 1074.

In block 1074, the method 1050 determines whether there is additional need or use or the AI therapist assistant 150. If not, the method 1050 is complete. On the other hand, if there are additional tacit the AI therapist assistant 150 can perform, the method 1050 returns to block 1058 and proceeds through blocks 1062, 1066, 1070 to determine other tasks that the AI therapist assistant may perform. Thus, the method 1050 allows the AI therapist assistant 150 to be active in the background and selectively be reinitiated to perform any groups of tasks whether they be during detox, pre-session, in-session, or post-session.

In some implementations, the method 1050 described above with reference to FIG. 10B may also include additional tracking and analysis steps not shown. For example, execution of individual assignments by the AI therapist assistant 150 can be tracked for completion, as well as evaluated for performance of the patient/user 106 in completing the task. This information can be provided to the AI hybrid care management server 120 to modify workflows or change the tasking framework. For example, based upon the patient/user 106 answers and completion of the assignments by the AI therapist assistant 150, the AI hybrid care management server 120 may notify staff within an EHR for the user on the AI hybrid care management server 120 and show the GUI of assigned and completed tasks in FIG. 19A, or connect the assignments to communication (SMS, email or AI phone calls) that would alert the clinical staff if there needs to be immediate follow-up to a user 106 based on those responses. For example, if a client had a relapse, was suicidal or needed to have a medication refilled immediately—the clinician 146 would be alerted in the EHR, SMS, email or an AI based phone call that could summarize the issue for the clinician 146 so he or she could take the appropriate action.

In some implementations, the method 1050 described above with reference to FIG. 10B may also be modified to provide user interfaces for creating programs where a “program” is a set of predefined set assignments and/or scheduled engagements organized in oriented at longitudinal care format. The above principles of the present disclosure may be modified to show, schedule and assign any of the elements of that longitudinal care format. For example, a virtual “Guide” or “Facilitator” could be created that cooperates with the AI therapist assistant 150 (e.g., is a separate bot or agent) or is incorporated into the AI therapist assistant 150 that will guide the user through the “Program”-enable the ability to save configurable, scheduled interventions, and therefore the “Program” may be defined and configured. The AI hybrid care management server 120 may be adapted with the ability to save multiple “Programs” to choose from or customize one.

The execution of the method 1050 of the present disclosure provides various types of interactions and engagements to produce different features of the AI engagements, described herein as user stories. Three example engagements are described below by way of example. A first example describes the management of artificial intelligence engagements (“AI Engagement Assignment Management”). Healthcare providers are enabled to create, manage, and track AI-powered patient engagement assignments through the execution of method 1050, including homework, pre-session warm-ups, and readiness checklists with comprehensive lifecycle management. An example user story may include an assignment type selection interface that enables a healthcare provider, to choose between different assignment types so that they can create the appropriate engagement for their patient's specific therapeutic needs. Some features may include enabling a provider to select from three assignment types: Homework Assignment, Pre-Session Warm Up, or Pre-Session Readiness Checklist. A selection interface appears as modal when clicking ‘Add New Assignment’ button. Each assignment type has distinct form fields, validation rules, and content libraries. Assignment type selection determines available therapeutic modules and timing options. Interface provides clear visual distinction between assignment types. In this example, a selection can be cancelled without creating assignment.

Another example user story may include a homework assignment creation workflow. For example, a healthcare provider may want to create comprehensive homework assignments for patients so that they can engage with therapeutic content between sessions with proper scheduling and follow-up. Features may be included, such as the ability for a provider to select multiple homework modules from therapeutic content library using searchable modal, ensuring that a provider must set first call attempt date using calendar date picker, must set call attempt time using dropdown time picker defaulting to 8:00 am, must specify number of additional attempts (1-99 numeric input), and must set specific time for additional attempts using time picker, in an implementation. A provider may also add optional notes in text area field. In one implementation, all required fields must be completed before saving (marked with red asterisks). A save button is visually disabled and non-clickable until all required fields are filled. Form validation prevents submission of incomplete assignments. Successfully saved assignments appear in Current Assignments section, in an implementation.

Another example user story is pre-session warm up assignment creation. For example, a healthcare provider may want to create pre-session warm-ups so that patients are therapeutically prepared before their scheduled appointments. Example features may include enabling a provider to select multiple warm-up modules from therapeutic content library, requiring that a provider specify a number of minutes before appointment (e.g., 1-999 numeric input), enabling a provider to choose application scope: Specific appointment or Appointment Type, and for specific appointments: enabling a provider to view and select from next 5 upcoming appointments with ‘Add more’ functionality, for appointment types: enabling a provider to select multiple appointment types from comprehensive list. An appointment type selection includes ‘All appointment types’ option that disables individual selections, in an implementation. Additionally, a provider may add optional notes. One feature may ensure that all required fields must be completed before saving. A form may be provided that shows appropriate validation and visual feedback, in an implementation.

Yet another example user story that may be implemented to add additional features through a workflow includes pre-session readiness checklist management. For example, a healthcare provider may want to create readiness checklists so that patients complete essential clinical screenings and assessments before appointments. Example features may include enabling providers to select from specific readiness checklist items: Suicide Assessment, PHQ-2, Anxiety Screen, Medication Compliance, Medication Refill, Habits Update, Behavior, Key Concerns. A provider may be required to specify a number of minutes before appointment (e.g., 1-999) that the checklist should be presented to a patient, for example. In an implementation, providers can apply a particular pre-session readiness checklist to specific appointments from upcoming appointments list, to multiple selected appointment types, and a provider can select an ‘All appointment types’ option. In one implementation, a provider can add optional notes for clinical context. In an example implementation, all required fields must be completed before saving and readiness checklist assignments may be provided for display with distinct green badge styling.

Another example user story that may include additional features that are configurable through the workflow builder is a dynamic content selection modal system. For example, a healthcare provider, may want to search, select, and organize therapeutic content so that they can customize assignments precisely for patient needs with proper content management. A modal opens when selecting assignment content with dynamic title based on assignment type, in an implementation. Then, the modal displays ‘Select AI Patient Homework’, ‘Select Pre-Session Warm Up’, or ‘Select Readiness Checklist Items’. A provider can search through available assignments using real-time search functionality. Additionally, a provider can select multiple assignments using checkbox interface, can reorder selected assignments via drag and drop with visual feedback, and can see previously used assignments with ‘Previously Used-[date]’ badges. Selected assignments appear in right panel with numbered order indicators, in an implementation. A provider can remove selected assignments from selection. The user interface may include an “OK” button that confirms selection and closes modal as well as a “Cancel” button discards changes, in an implementation. The modal is responsive and handles large content libraries efficiently.

Another example user story is assignment lifecycle and state management in which a healthcare provider may want to edit, delete, and track assignment status so that they can adjust treatment plans dynamically as patient needs evolve. A provider can edit existing assignments using Edit button with pencil icon. Edit form pre-populates with all existing data including selected content, timing, and notes. Save button changes to ‘Update Assignment’ when in edit mode. A provider can delete assignments with confirmation dialog to prevent accidental deletion. A confirmation dialog shows assignment details and requires explicit confirmation. Edited assignments show ‘Updated’ timestamp alongside creation date. Deleted assignments are immediately removed from current assignments list. Assignment state changes are reflected in real-time across the interface. In this way, edit and delete operations maintain data integrity.

Another example user story for AI Engagement management includes an assignment organization and display system. A healthcare provider may want to view current and completed assignments in an organized, filterable manner so that they can efficiently track patient progress and assignment history. Features include having current assignments that are displayed with creation date, timing details, and activity lists, completed assignments that are shown in a separate section with completion dates, an organization toggle that allows switching between mixed and type-organized views and a toggle that is checked by default to organize assignments by type. When organized by type, assignments are grouped under H2 headers: ‘Homework Assignments’, ‘Pre-Session Warm Ups’, ‘Pre-Session Readiness Checklists’, in an implementation. Each assignment shows appropriate type badge with distinct colors (blue for homework, purple for warm-up, green for readiness). Assignment cards display comprehensive details: activities, timing, appointment types, and notes. In one implementation, bold headers for ‘Created’ and ‘Timing’ sections are provided to improve readability and empty states are handled gracefully when no assignments exist.

Another user story for AI Engagements include an assignment form that includes user experience optimization. A healthcare provider may want an intuitive assignment creation experience so that they can efficiently create assignments without workflow interruptions. In an implementation, an assignment form appears at top of page when adding new assignment for immediate visibility. The form is contained in highlighted gray background container for visual distinction. The form includes cancel functionality to close without saving changes. The form title dynamically changes based on assignment type being created. The form validation provides real-time feedback on required field completion. Time pickers default to 8:00 am and open positioned at 8:00 am to minimize scrolling. All form interactions provide appropriate visual feedback and hover states. Additionally, the form layout is responsive and maintains usability across different screen sizes.

An additional user story includes seed data and testing infrastructure. For example, a system user may want realistic test data available so that they can evaluate system functionality and see examples of all assignment types in various states. In an implementation, a system includes sample current assignments for all three assignment types, sample completed assignments showing assignment lifecycle, and test data includes realistic therapeutic content, dates, and clinical notes. Additionally, previously used assignment detection works with sample data. Sample appointments are available for specific appointment selection and test data demonstrates all major features and edge cases. In this way, data is clinically realistic and appropriate for healthcare context.

A second example engagement includes healthcare navigation and system integration. A comprehensive, secure navigation system may be provided, in an implementation, for healthcare providers to access different aspects of patient care management with proper authentication and system organization. User Stories may include AI engagements navigation integration. For example, a healthcare provider may want to access AI engagement assignment functionality through intuitive navigation so that it integrates seamlessly with their clinical workflow. Features include having navigation that includes ‘Assign AI Engagements’ tab positioned between Billing and AI Conversations, a tab that maintains consistent styling with other navigation items using system design tokens, an active state that is clearly indicated with blue underline and proper contrast, tab text that fits properly within navigation bar without wrapping or truncation, navigation that is responsive and works across different screen sizes, and where tab switching preserves application state appropriately.

Another user story includes system authentication and security where a system administrator wants to protect the healthcare system with secure authentication so that only authorized users can access sensitive patient data and clinical tools. Example features include a login screen that appears before accessing healthcare navigation with professional styling, a password field that accepts ‘anonymous’ as valid credential for demo purposes, a launch button that submits authentication with proper form handling, an enter key that triggers authentication for improved user experience, a successful login that reveals complete healthcare navigation interface, failed login attempts show appropriate error messaging, and an authentication state that is maintained throughout session.

Another example user story includes a comprehensive navigation system where a healthcare provider wants access to all clinical tools through a unified navigation system so that they can efficiently manage patient care across different domains. Features of the comprehensive navigation system include, in one implementation, navigation that includes all clinical tabs: Overview, Treatment Plan, Client Data Library, Schedule, Encounters, Client Profile, Documentation, Referral History, Billing, Assign AI Engagements, AI Conversations, navigation that maintains consistent visual hierarchy and spacing, an active tab state that is clearly communicated with visual indicators, navigation that is keyboard accessible for users with disabilities, tab switching that is smooth and maintains application performance, and navigation that works consistently across different browsers and devices.

A third example engagement includes user experience and system optimization. The overall user experience is enhanced through intuitive form controls, comprehensive validation, visual feedback systems, and complete project documentation for stakeholders and development teams. User stories include an advanced time selection interface where a healthcare provider user is provided with an intuitive time picker so that they can quickly select appointment times without excessive scrolling or workflow interruption. The features include, in an implementation, a time picker that defaults to 8:00 am for all time fields across the application, a dropdown that opens positioned at 8:00 am in the list for immediate access to common times, times that are displayed in 12-hour format with am/pm indicators for clarity, and where 15-minute increments are available from 12:00 am to 11:45 μm for comprehensive coverage. Additionally, users can scroll up for earlier times, down for later times with smooth scrolling, a time picker maintains consistent styling with other form controls, a selected time is clearly displayed in the input field, and the time picker works consistently across all assignment types.

Another user story includes a comprehensive form with validation and visual feedback. A healthcare provider may want clear, immediate visual feedback on form requirements so that they understand exactly what needs to be completed before saving assignments. Features include having required fields that are marked with red asterisks (*) for immediate identification, a save button that is visually disabled with gray styling when required fields are missing, a disabled save button that shows not-allowed cursor to indicate non-interactive state, a save button that enables with proper styling only when all required fields are completed, a notes field that remains optional for all assignment types with clear visual indication, a form validation that works consistently across all three assignment types, error states are handled gracefully with appropriate messaging, and the form provides positive feedback when validation requirements are met.

Further, yet another user story includes project documentation and development support. For example, a project stakeholder and development team member may want comprehensive, accessible project documentation so that they can understand system capabilities, development progress, and implementation requirements. Features include, in one implementation, “Epics” & “Stories” button(s) that are available in navigation bar for easy access, a modal that displays comprehensive project documentation with professional formatting, documentation that includes detailed epics with complete user stories covering all functionality, where each story includes specific, testable acceptance criteria for engineering implementation. Additionally, stories, in an implementation, may show completion status with clear visual indicators (completed/in-progress), documentation covers all Assign AI Engagements functionality comprehensively, epic descriptions provide clear business context and value proposition, documentation is organized logically for both technical and non-technical stakeholders, the modal is responsive and provides good reading experience, and documentation can be easily updated as new features are added.

Another user story includes assignment type badge and visual hierarchy. A healthcare provider may want clear visual distinction between assignment types so that they can quickly identify and organize different kinds of patient engagements. Example features include, in an implementation, homework assignments that display with blue badges labeled ‘Homework Assignment’, pre-session warm ups that display with purple badges labeled ‘Pre-Session Warm Up’, readiness checklists that display with green badges labeled ‘Pre-Session Readiness Checklist’, badge colors that maintain sufficient contrast for accessibility compliance, badge styling that is consistent across current and completed assignment sections, visual hierarchy that clearly distinguishes between assignment types when organized, and badge text that is concise but descriptive for immediate recognition.

Yet another user story includes default organization and user preferences. A healthcare provider may want assignments organized by type by default so that they can efficiently review different categories of patient engagements without additional configuration, in an implementation. Features may include an organization toggle that is checked by default when accessing “Assign AI Engagements” interface. Additionally, assignments may be automatically grouped by type: Homework, Pre-Session Warm Up, Readiness Checklist. Users can still toggle to mixed view if preferred for their workflow, in an implementation. An organization preference may affect both current and completed assignments sections, in an implementation. Additionally, a toggle state that provides clear visual feedback about current organization mode is provided in an implementation. In this way, a default organization improves clinical workflow efficiency.

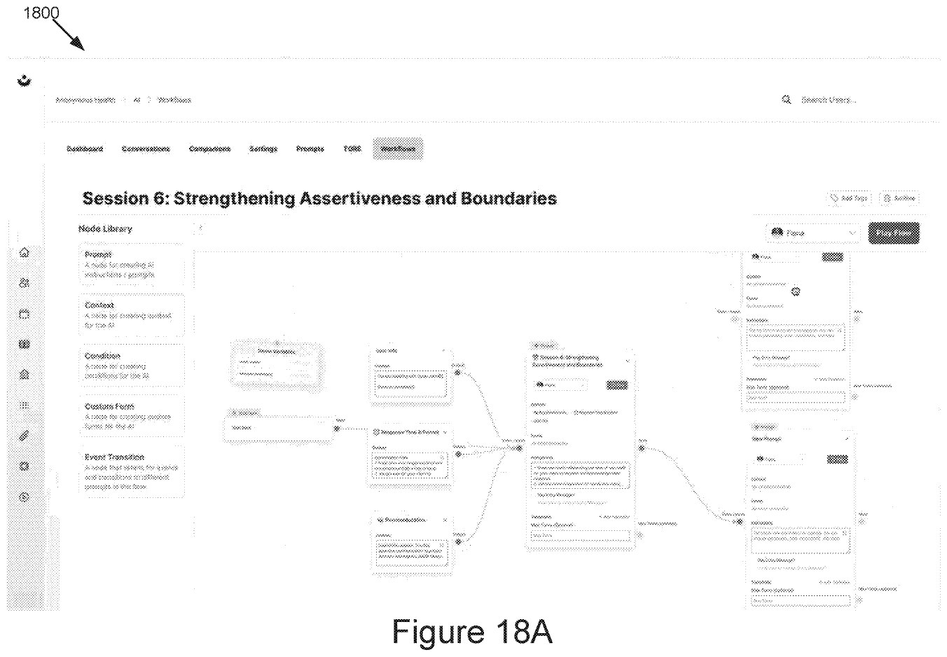

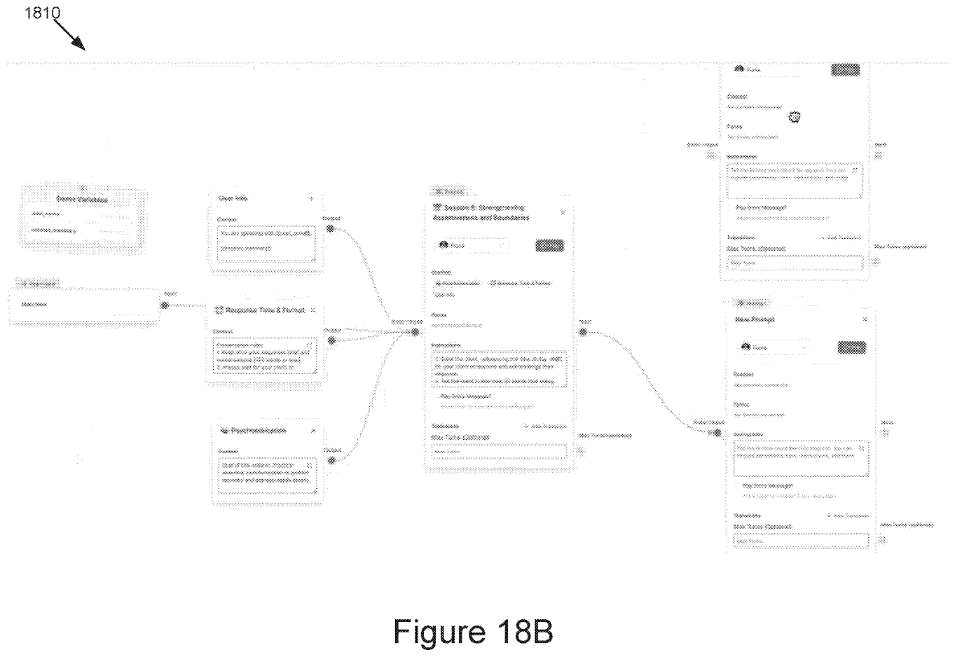

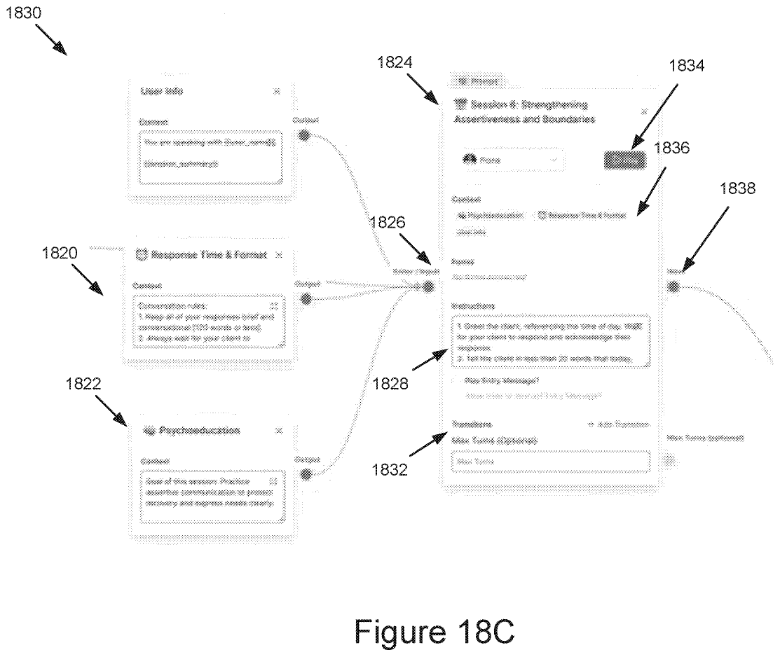

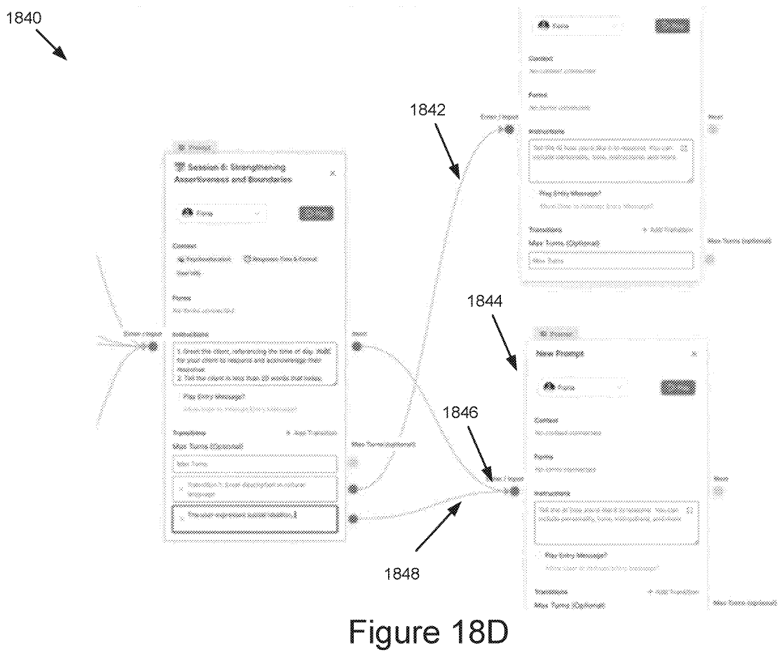

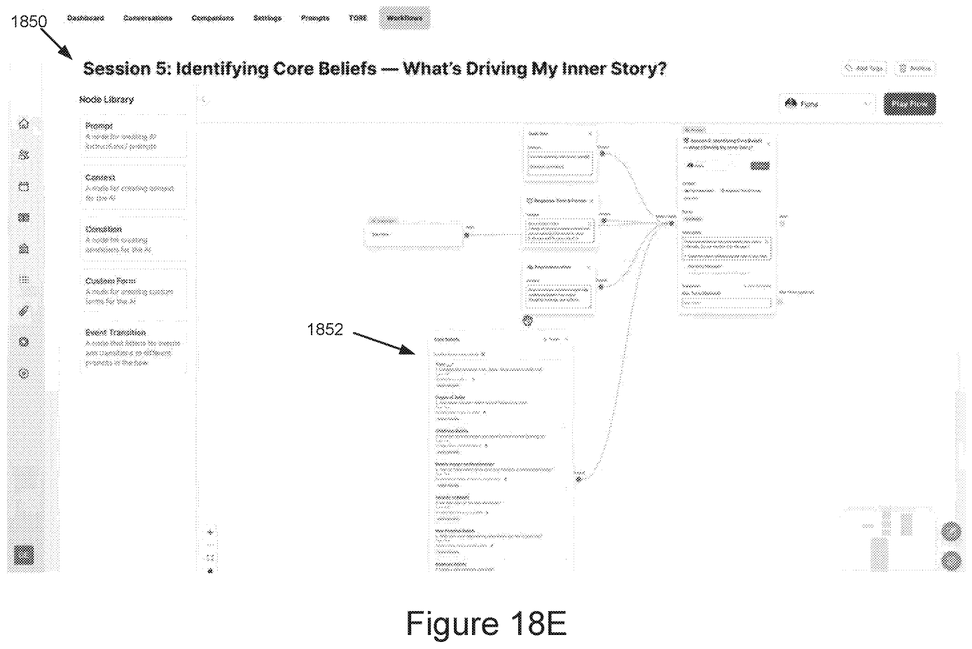

FIG. 11 is an example flowchart diagram of a method of providing workflow-based configuration of agentic AI sessions in mental health treatment. The AI Therapist Assistant is enabled by the workflow-based configuration of agentic AI sessions as described through method 1100. Method 1100 may begin with user input being received 1102 from a supervising user through a workflow data management interface. For example, a supervising user may operate a software application to generate a workflow-based configuration of an AI session using a graphical user interface as shown in the example screens of FIGS. 18A-18E.

A configuration of an AI agentic workflow associated with a patient user is determined 1104 based on the received user input.

A plurality of data items associated with the configuration of the AI agentic workflow are retrieved 1106 based on the received user input from the supervising user.

One or more AI models are then selected 1108 to request a series of AI generated questions and/or responses based on the AI agentic workflow.

The plurality of data items associated with the configuration of the AI agentic workflow is provided 1110 to the selected one or more AI models.

A request for the series of AI generated questions and/or responses from the selected one or more AI models is sent 1112 based on one or more responses received through a therapeutic chat interface from the patient user.

Based on the one or more responses received through the therapeutic chat interface, the series of AI generated questions and/or responses are received 1114 from the selected one or more AI models.

Then, based on the configuration of the AI agentic workflow, the series of AI generated questions and/or responses are provided 1116 through the therapeutic chat interface.

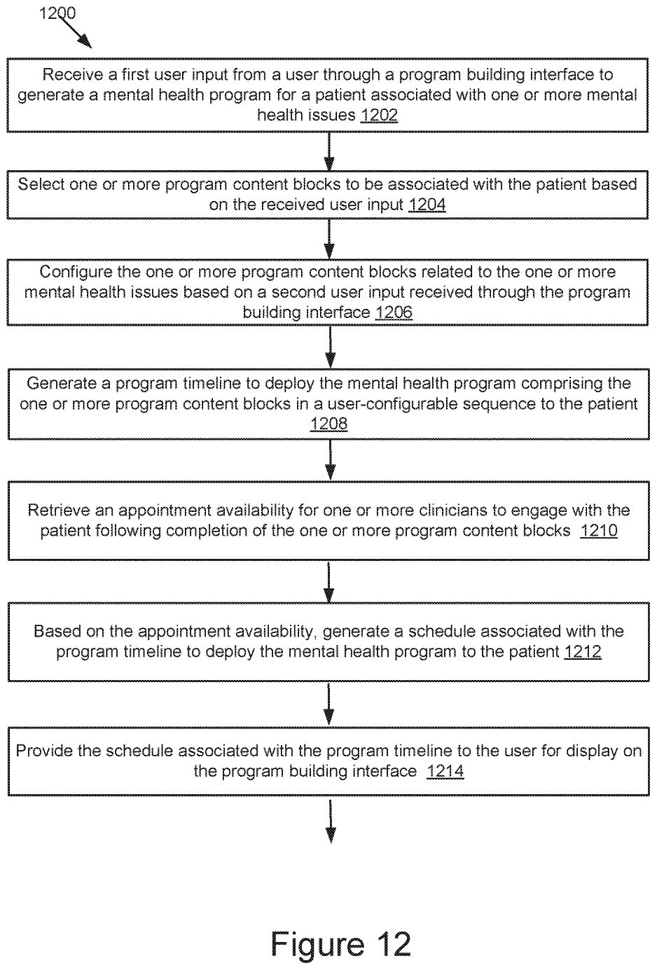

FIG. 12 is an example flowchart diagram of a method of operation of building a program in accordance with the present disclosure. Referring now to FIG. 12, one implementation for a method 1200 executed by the AI Hybrid Care Management Server 120b and/or system 100b will be described. The method 1200 begins by receiving a first user input from a user through a program building interface to generate a mental health program for a patient associated with one or more mental health issues at step 1202. Next, the method 1200 selects 1204 one or more program content blocks to be associated with the patient based on the received user input. In one example, program content blocks available for selection may be presented in a user interface (e.g., the program building interface) to the user. In another example, the user may create a new program content block through the user interface in coordination with a workflow builder. In yet another example, the user may select a previously configured program that comprises one or more program blocks to be associated with the patient at step 1204. The method 1200 continues at step 1206 where the one or more program content blocks related to the one or more mental health issues are configured based on a second user input received through the program building interface. For example, the one or more program content blocks may be configured to be deployed in a certain ordered sequence, where a first program content block relates to decisional balance and building the confidence to change, followed by a second program content block relating to enhancing self-efficacy with strengths-based reflection, then a third program content block relating to setting intentions and following through. Through the second user input, the one or more program content blocks may be reordered, new program content blocks may be added and/or created, some program content blocks may be removed, a cadence of the program may be configured, such as starting the program 15 minutes after the program start date/time, configuring a number of additional attempts if the client patient does not respond, modifying the program name, program description, program tags, program status, and default schedule, such as triggering the program from a residential admission date, as well as adding additional AI engagements that encompass other programs such as a pre-session readiness checklist, a pre-session warm up, homework assignment(s), post-session activities, customized programs, and so forth.

Method 1200 continues at step 1208 where a program timeline is generated to deploy the mental health program comprising the one or more program content blocks in a user-configurable sequence to the patient. The AI therapist is configured to present the mental health program to the patient through a software application or a phone call, in some implementations. As an example, a program timeline may include a recurring appointment with the AI therapist where the program is deployed. The program timeline may be generated 1208 through determining a calendar of dates and times when the AI therapist will engage with the patient through a mobile device or computer in a software application in a therapeutic chat interface and/or through a phone call or video call. In some implementations, a program may be referred to an AI engagement because the AI therapist and/or AI companion is used to deliver the program content. At step 1210, an appointment availability for one or more clinicians is retrieved to engage with the patient following completion of the one or more program content blocks. Because a session may be hybrid, meaning that an AI therapist and a human therapist may both be involved in the session, the availability of the human therapist is necessary to schedule the session, in some implementations. Based on the appointment availability, a schedule associated with the program timeline is generated 1212 to deploy the mental health program to the patient. Finally, the method 1200 provides 1214 the schedule associated with the program timeline to the user for display on the program building interface. In some embodiments, the AI therapist assistant 150 may complete some or all of the steps of method 1200. The above are just a few examples of what can be performed with program builder 904 and/or the AI therapist assistant 150.

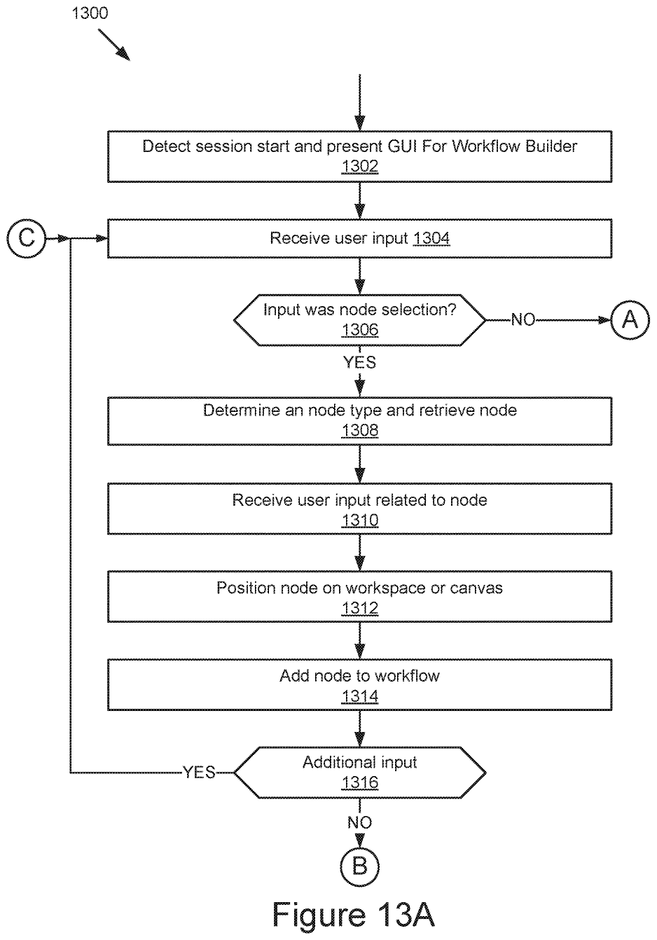

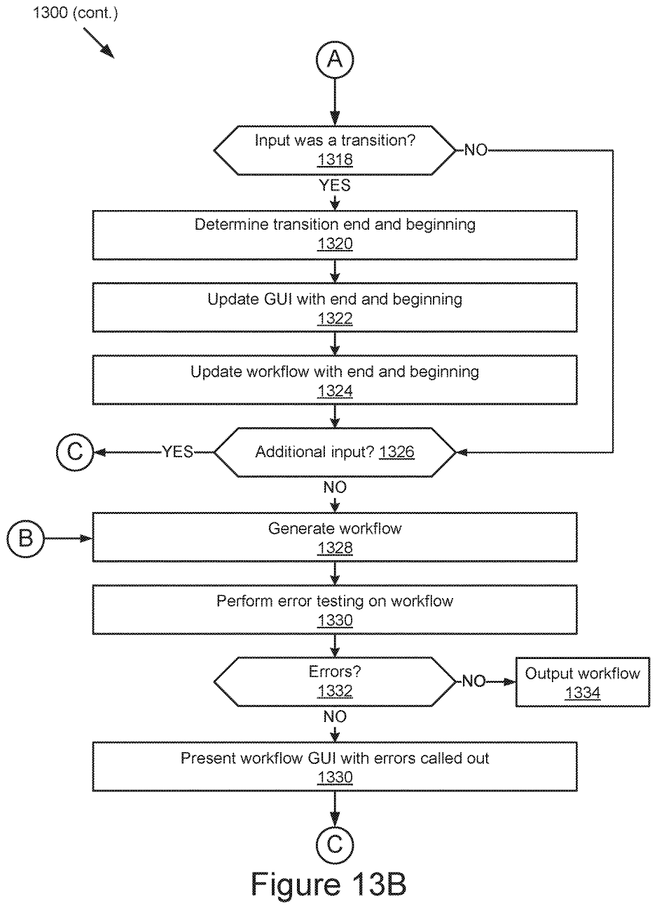

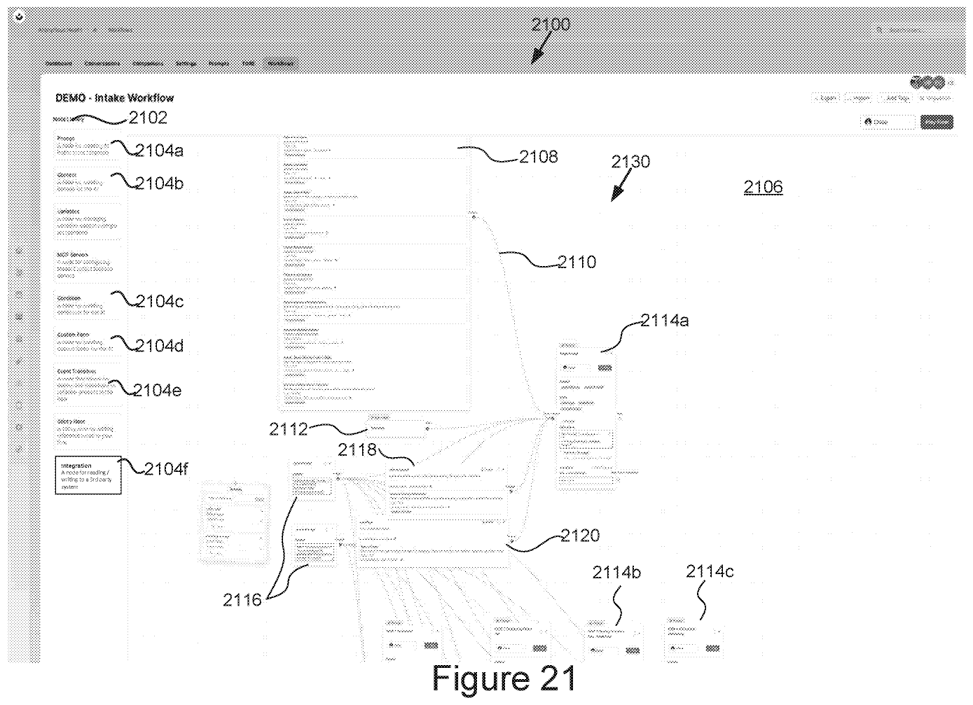

FIGS. 13A-13B are an example flowchart diagram of a method of operation of workflow builder in accordance with the present disclosure. Referring now to FIGS. 13A and 13B, the method 1300 of operation of workflow builder 902 will be described. The method 1300 begins by detecting 1302 a session start and presenting a user interface for the workflow builder 902. Such an example user interface will be described in more detail below with reference to FIG. 21. Next, the method 1300 receives 1304 user input. The user input can be creation of any one of the nodes described above, the addition of the transition, input variable the references, or input of other information only to workflow. The method 1300 continues by determining whether the input was a node selection. If not, the method 1300 transitions to block 1318 as will be described below. On the other hand, if the method 1300 determines in block 1306 that the input was the selection of the node, the method 1300 continues in block 1308. In block 1308, the method 1300 determines a node type and retrieves that type of node. For example, the node can be retrieved from the workflow library 1518. In an implementation, the workflow library 1518 includes any number of workflow templates which may include one or more prompt nodes, tool calls, forms, if/then conditions as well as accompanying scenarios and/or graders. These are all designed to deliver an interactive conversational AI experience to the patient aligned to the parameters of intended clinical interactions and/or goals. Next, the method 1300 receives 1310 user input related to the node identified in block 1308. The method 1300 continues by positioning 1312 the node on the workspace or canvas 1506. Next, the method 1300 adds 1314 the node to the workflow. In some implementations, the updated version of workflows is also stored in the workflow library 1518. This essentially completes the creation or updating of a node in the workflow. Next, the method 1300 determines 1316 whether there is additional from the user. If so, the method 1300 returns to block 1304 to receive the additional user input. If not, the method 1300 transitions from block 1316 of FIG. 13A to block 1328 of FIG. 13B.

Referring now to FIG. 13B, the method 1300 continues to determine 1318 whether the input from block 1304 was a transition between two nodes. If not, the method 1300 proceeds to block 1326 as will be described below. However, if the input from block 1304 was a transition between nodes, the method 1300 determines a beginning node and an ending node for the input transition. The method 1300 continues by updating the user interface with a line connecting the beginning node and the ending node determined in block 1320. Then the method 1300 updates 1324 workflow to add a transition between the two nodes. In some implementations, the updated version of workflows is also stored in the workflow library 1518. The method 1300 continues to determine 1326 whether there is additional input from the user. If so, the method 1300 proceeds to block 1304 of FIG. 13A. If not, the method 1300 continues by generating 1328 the workflow including its nodes, variables, transitions, and other information. Then the method 1300 performs 1330 error testing on the workflow. In block 1332, the method 1300 determines whether any areas are detected in the execution of the workflow. If not, the workflow is output 1334 or used in the AI hybrid care management server 120. On the other hand, if errors were detected in block 1332, the method 1300 proceeds to present the workflow in the user interface with the errors called out. After block 1330, the method transitions back to block 1304 to receive additional input to correct the errors. FIGS. 13A and 13B are merely one example of the implementation of the method of the present disclosure. Other implementations may modify the order in which the steps of the processing are performed, add additional steps, or eliminate steps, all of which are included within the scope of the present disclosure.

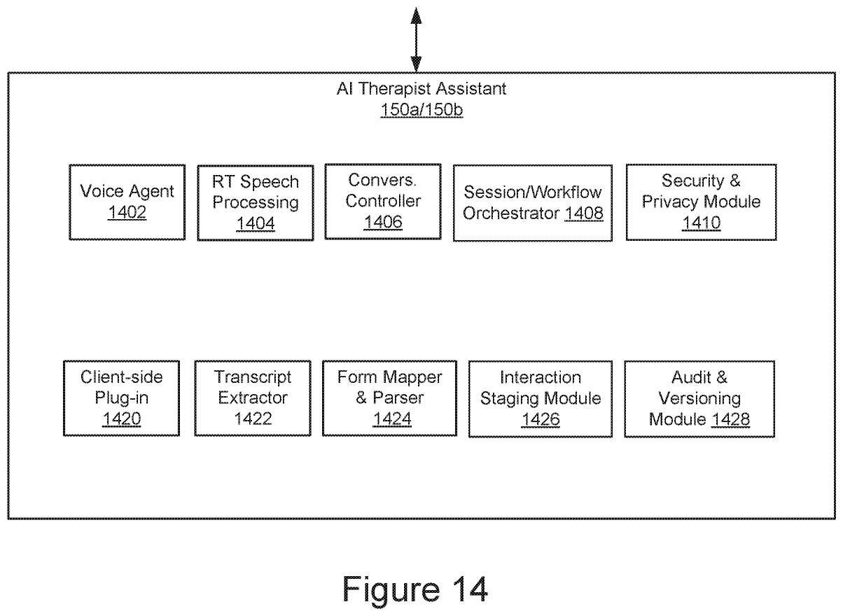

FIG. 14 is a block diagram illustrating an implementation of the AI therapist assistant in more detail. Referring now to FIG. 14, the AI therapist assistant 150 according to some implementations will be described. In some implementations, the AI therapist assistant 150 comprises a voice agent 1402, a real-time speech processing module 1404, a conversation controller 1406, a session/workflow orchestrator 1408, a security and privacy module 1410, a client-side plug-in 1420, a transcript extractor 1422, a form mapper and parser 1424, an interaction staging module 1426, and an audit and versioning module 1428. These components of the AI therapist assistant 150 are coupled for communication, interaction, and cooperation with each other and with the other components of the AI hybrid care management server 120b.

As noted above, different configurations of these components and/or elements of the AI therapist assistant 150 may be operational on the AI hybrid care management server 120b alone, the clinician device 145a alone, the client device 115a alone, or any combination of different component on either the AI hybrid care management server 120b, the clinician device 145 or the client device 115. In one preferred example implementation, the AI therapist assistant 150 is a lightweight integration of the AI therapist assistant 150 to a 3^{rd} party EMR 180: In such an implementation, the AI therapist assistant 150(1) gets context from and read/write to the EMR/virtual meeting platform 170, and (2) the AI Therapist 150 engages the patient/therapist in the session. FIG. 14 is a block diagram illustrating an implementation of various implementations of read-write to the EMR for purposes of (1) providing context to the AI Therapist Assistant 150, and/or (2) recording important information to the medical record (Session Monitoring Module 128), and/or (3) triggering a crisis or escalation workflow, and/or (4) triggering a Task to be assigned to one or more persons, roles or groups, and/or (5) scheduling follow-up engagements, and/or (6) triggering a workflow instance of any number of defined workflow templates. A browser plug in which reads the Document Object Model (DOM) or otherwise interprets a 3^{rd} party EMR web interface and reads and writes to the 3^{rd} party EMR. Additionally, there exists an API integration of the AI Hybrid Care Management Server 120b with a 3^{rd} party EMR Management Module 180.

Various implementations of enabling an AI Therapist Assistant are also supported on one platform via an AI Hybrid Care Management Service 120b to engage via voice or text with a therapist and/or patient via a separate Virtual Meeting Platform 160. For example, the AI Hybrid Care Management Service 120b reads the schedule and dial-in information for appointments via the read-write methods to the 3^{rd} party EMR 180 and/or the virtual meeting platform 160 and/or another source of information on (i) the appointment start and end times, (ii) the patient and therapist identification information, and (iii) the information needed to access and authenticate into the appointment (e.g., via dial-in or web VoIP engagement), and then uses this information to have the AI Therapist Assistant 150 join the session as a participant either via web VOIP engagement or via phone dial-in. The therapist enters the web address and/or phone number and/or appointment start time and/or patient identifying information of an appointment to the Therapeutic Chat Interface Application 110b and thereby causes the AI Therapist Assistant to dial in or join the session via the Session Routing Module 122.

The voice agent 1402 may include software and/or logic to provide functionality for generating a software agent that acts like a participant of any session or virtual meeting between a clinician 146 using the clinician device 145 and a user 106 using the client device 115. Once a session is started on the virtual meeting platform 160, the voice agent 1402 joins the session just as any participant would. The voice agent 1402 connects as a pseudo-participant at the scheduled telehealth start time of a session. The voice agent 1402 is able to access any capability of the virtual meeting platform 160 and/or the meeting assistant 170 associated with the session. In some implementations, the connection by the voice agent 1402 is established via a WebRTC-based voice channel or Session Initiation Protocol (SIP) through telephony. The voice agent 1402 maintains full-duplex audio capability and receives voice and/or video input from both clinician 146 and the user 106. Responsive to communication with the other components of the AI therapist assistant 150, the voice agent 1402 can also access any functionality of the virtual meeting platform 160 and/or the meeting assistant 170. For example, the voice agent 1402 can record any part of the session, transcribe any part of the session, or send instructions to the virtual meeting platform 160 or the meeting assistant 170 to perform such actions enabled by them. In some implementations, the voice agent 1402 provides raw audio or video signals to the other components of the AI therapist assistant 150 for processing in lieu of the virtual meeting platform 160 or the meeting the system 170. The voice agent 1402 is coupled for communication and interaction with the other components of the virtual meeting platform 150 and other elements of the system 100.

The real-time (RT) speech processing module 1404 may include software and/or logic to provide functionality for receiving audio and/or video signals and converting them to text. In some implementations, the real-time speech processing module 1404 runs a speech-to-text pipeline with voice activity detection (VAD) to capture utterances. In some implementations, the real-time speech processing module 1404 sends transcribed segments to a language model (not shown) which processes conversational context and therapeutic intent. In some implementations, the real-time speech processing module 1404 outputs are converted to synthetic speech and played back with low latency (<300 ms) to maintain fluid interaction. The real-time speech processing module 1404 is coupled to receive raw audio signals from the voice agent 1402. The real-time processing module 1404 is coupled for communication and interaction with the other components of the virtual meeting platform 150 and other elements of the system 100.

The conversation controller 1406 may include software and/or logic to provide functionality for controlling when the AI therapist the system 150 provides output in the virtual meeting. In some implementations, the conversation controller 1406 includes a voice activity detection (VAD) that ensures proper turn-taking in conversation. The VAD detects end-of-speech to avoid overlap or interruption. In some implementations, the conversation controller 1406 also dynamically adjusts voice tone, pacing, and content based on session progress and therapeutic goals. The conversation controller 1406 is coupled to the voice agent 1402 to provide output which the voice agent 1402 in turn interjects into the virtual meeting or session. The conversation controller 1406 is also coupled for communication and interaction with the other components of the virtual meeting platform 150 and other elements of the system 100.

The session/workflow orchestrator 1408 may include software and/or logic to provide functionality for control, organization and flow of a therapy session. The session/workflow orchestrator 1408 is a manager component that initializes the audio pipelines, links them to the language model, and monitors session timing, control signals, and progress of the therapy session. The session/workflow orchestrator 1408 is configured to control other elements of the AI therapist assistant 150 including the voice agent 1402, the real-time speech processing module 1404, and the conversation controller 1406. In some implementations, the session/workflow orchestrator 1408 is also coupled for communication and interaction with the other components of the virtual meeting platform 150 and other elements of the system 100. In some implementations, the mechanism for defining workflows in the session/workflow orchestrator 1408 includes the ability for an operational or clinical expert to organize software-defined modular nodes into executable workflows, wherein the types of nodes may include but are not limited to: (i) prompts, (2) context that may be retrieved from one or more systems and fed to one or more nodes or to the overall workflow context, (3) conditions which define if/then rules for progressing through the workflow, (4) custom forms which may define information to be read from a data source and/or written to a data source, (5) tasks which may be assigned to one or more individuals or groups of individuals, including based on role definitions of the individuals, and which can be assigned and instantiated within one or more systems, and/or (6) transitions which are nodes that may be managed by either deterministic software examining progress through a workflow state machine and outputting events, and/or an AI agent examining this data at specified times or throughout a workflow and outputting events, wherein the events may be used to activate other parts of the workflow. One example of a workflow is shown in FIG. 21.

The security and privacy module 1410 may include software and/or logic to provide functionality for protecting and securing the data received and sent by the AI therapist assistant 150. The security and privacy module 1410 cooperates with the other components of the virtual meeting platform 150 to encrypt the audio/text data flows into and out of the AI therapist the system 150. For example, the security and privacy module 1410 cooperates with the voice agent 1402 to encrypt any transmissions to or from it and the virtual meeting platform 160. Similarly, the security and privacy module 1410 encrypt transmissions between the AI therapist assistant 150 and other systems, for example, EMR storage systems. In some implementations, the security and privacy module 1410 may also optionally log call recording and transcriptions per HIPAA-level compliance for audit and review. The security and privacy module 1410 is also coupled for communication and interaction with the other components of the virtual meeting platform 150 and other elements of the system 100 to ensure privacy and security.

The client-side plug-in 1420 may include software and/or logic to provide functionality for making any portion of the AI therapist assistant 150 operational on a device other than the AI hybrid care management server 120. For example, the client-side plug-in 1420 makes the AI therapist assistant 150 operational on either the client device 115 or the clinician device 145 as shown above in FIG. 9. In one implementation, the client-side plug-in 1420 is a browser extension that activates when the EMR interface is loaded in a session browser tab based on URL or DOM patterns. In a second implementation, client-side plug-in 1420 is platform-specific OS application. In this second implementation, the application runs within the native OS docking system and would manage the browser with browser development tools protocol. The third implementation, the client-side plug-in 1420 is an application with binary code of a browser to perform the browser functions and interact with the other components of the AI therapist assistant 150. For example, this implementation could be an application instance including chromium open-source code from Google. The client-side plug-in 1420 is also coupled for communication and interaction with the other components of the virtual meeting platform 150 and other elements of the system 100.

The transcript-driven extractor 1422 may include software and/or logic to provide functionality for generating text from any session in which the AI therapist the system 150 participates. In some implementations, transcript-driven extractor 1422 continuously fetches real-time transcribed text from the voice session (exposed via secure local API). The transcript-driven extractor 1422 may be implemented as a browser extension or an independent application. The transcript-driven extractor 1422 is coupled to the voice agent 1402 to receive raw audio signals for transcription. The transcript-driven extractor 1422 is also coupled for communication and interaction with the other components of the virtual meeting platform 150 and other elements of the system 100.

The form mapper and parser 1424 may include software and/or logic to provide functionality for automatically completing and submitting any form associated with a therapy session. In some implementations, the form mapper and parser 1424 uses a natural language processor (NLP) to parse transcript and extract relevant data fields (e.g., symptoms, medications, recommendations) from a therapy session. The form mapper and parser 1424 accesses a database (not shown) of forms associated with different conditions or circumstances that may occur in a therapy session for a patient. The form mapper and parser 1424 monitors the therapy session and cooperates with the real-time speech processing module 1404 to secure any data needed. In some implementations, the form mapper and parser 1424 includes a mapping layer that correlates parsed data with specific EMR form elements (e.g., notes fields, dropdowns, checkboxes). The form mapper and parser 1424 determines the form needed for completion, receives confirmation that the form should be completed from the interaction staging module 1426, then processes the text data from the session and populates it into the identified form and submits the form to a system outside of the AI therapist assistant 150. The form mapper and parser 1424 is coupled for communication and interaction with the other components of the virtual meeting platform 150 and other elements of the system 100 for performing its operations.

The interaction staging module 1426 may include software and/or logic to provide functionality for generating and presenting in the user interface suggestions of operations or steps, and confirmation of their execution. In some implementations, the interaction staging module 1426 suggests entries that appear in an extension UI overlay. The therapist reviews and approves the suggested entries before changes are executed. In one example, interaction staging module 1426 interacts with a browser extension that manipulates the EMR page DOM or API endpoints to fill in fields or trigger checkboxes. The interaction staging module 1426 is also coupled for communication and interaction with the other components of the virtual meeting platform 150 and other elements of the system 100.

The audit and versioning module 1428 may include software and/or logic to provide functionality for creating audit records of sessions in which the AI therapist assistant 150 participates. For sessions where the AI therapist assistant 150 is activated, the actions suggested and executed by the AI therapist assistant 150 are logged, time stamp, and a source transcript is generated. In some implementations, the audit and versioning module 1428 also determines associated agent confidence levels and records therapist approval. In some implementations, the audit and versioning module 1428 also archives logs as part of the AI therapist assistant 150, the AI hybrid care management server 120 or other destinations. The logs can be archived per session for compliance and dispute resolution. The audit and versioning module 1428 is also coupled for communication and interaction with the other components of the virtual meeting platform 150 and other elements of the system 100 or these auditing and versioning purposes.

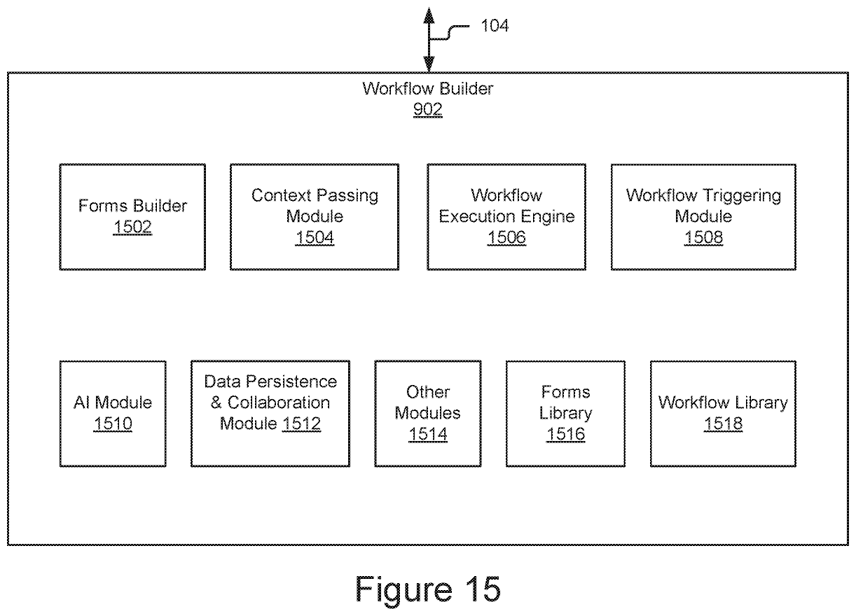

Referring now to FIG. 15, the workflow builder 902 according to some implementations is described. In some implementations, the workflow builder 902 comprises a forms builder 1502, a context passing module 1504, a workflow execution engine 1506, a workflow triggering module 1508, an artificial intelligence (AI) module 1510, a data persistence and collaboration module 1512, other modules 1514, a forms library 1516, and a workflow library 1518. These components of the forms builder 902 are coupled for communication, interaction, and cooperation with each other and with the other components of the AI hybrid care management server 120 and the data sources 135.

The operation of the workflow builder 902 and its cooperation with the other components of the AI hybrid care management server 120 will be described in more detail below and is particularly advantageous because it allows clinicians and others without technical backgrounds or programming experience to create AI Therapist Assistant content and related workflows including creation of a workflows or a form as discrete, configurable pieces of clinical content. The workflow builder 902 is a critical part of the backend of the AI hybrid care management server 120 because it allows the clinicians and administrators to customize and optimize the operation of the AI assisted mental health counseling to meet a myriad of different requirements. Using the workflow builder 902, a clinician can create the building blocks for mental health counseling, leveraged those building blocks into workflows, specify how context and other information is passed between nodes in the workflow, define how a workflow will be triggered or conditions that will initiate or trigger a workflow, and define the relationship between AI, human tasks, and custom forms.

In some implementations, the workflow builder 902 is a visual, node-based editor that enables construction of healthcare workflows from discrete building blocks. For example, the discrete building blocks or nodes include prompt nodes, context nodes, form nodes, condition/event nodes, variable nodes, and/or interaction nodes. A prompt node defines AI instructions and prompt configuration. A context node manages variables and contextual data relative to the workflow and/or a node. The form node generates dynamic forms for the provision of context or for input to be collected by an omni-channel AI and/or a human. The condition/event node handles branching logic, trigger transitions, and/or events. The variable node centralizes global state and provides variables that can be used globally across two or more workflows or locally only in one workflow. The interaction node is a node for reading or writing to a 3^{rd} party system. The workflow builder 902 with these nodes advantageously provides a low-code/no-code structure that enables non-technical users to compose complex workflows that blend AI and human tasks. A key element is the inclusion of executable if-then logic that provides explainability for AI-driven decisions. This is particularly important in regulated industries such as healthcare, where auditability and interpretability of LLM actions are critical. The workflow builder 902 is an extensible system for building AI-driven and human-in-the-loop clinical workflows. By treating forms, prompts, and context as composable nodes, and enabling real-time orchestration and collaboration, the platform enables creation of configurable “clinical content workflows” that go beyond static pathways. With event nodes for external triggers and if-then explainability logic, the workflow builder 902 provides both technical power and the transparency necessary for regulated healthcare use cases.

The forms builder 1502 may include software and/or logic to provide functionality for defining and creating a form, storing performing forms library 1516, and using a form as part of the workflow. For example, the forms builder 1502 produces dynamic forms that are tightly integrated into workflows. The forms builder 1502 produces dynamic forms that are a reusable, validated data collection tool that can be attached to AI or Human Task nodes, or used by the AI Therapist Assistant 150 to gather structured patient input (e.g., PHQ-2, custom proprietary assessments), and can be saved directly to AI hybrid care management server 120, the forms library 1516, or and published as real-time events for downstream workflow logic. This combination makes forms not just static data collectors, but executable workflow elements. Once created, a form can then be attached to a Human Task Block or an AI Task Block for completion. The AI Task Block may do its best to complete the form automatically based on available context, or its Task may be to engage a human to complete the form (e.g. Task assigned to AI Therapist Assistant to engage a patient to complete a PHQ-2 or a Custom Form created by a third party that may be proprietary to a third party). The forms builder 1502 is coupled for communication and interaction with the other components 1504, 1506, 1508, 1510, 1512, 1514, 1516, and 1518.

The context passing module 1504 may include software and/or logic to provide functionality for passing context between elements or node in a workflow. For example, the workflow builder 902 includes several mechanisms for passing context which are handled by the current processing module 1504. The context passing module 1504 supports edge-based connections (data flowing along visual links); centralized variable system (global state accessible anywhere in the workflow); session-based context updates (real-time updates, ensuring AI and human participants have the latest state). The context passing module 1504 may further receive context from data in forms as they are loaded in the context of workflow execution or as the forms are completed by a human and/or AI, including via a Human Task Block or an AI Task Block. This is particularly advantageous because it allows downstream nodes including AI prompts and forms to dynamically adapt based on prior patient input, clinician activity, or AI analysis. The context passing module 1504 is coupled for communication and interaction with the other components 1502, 1506, 1508, 1510, 1512, 1514, 1516, and 1518.

The workflow execution engine 1506 may include software and/or logic to provide functionality for executing state machines and orchestrating for cooperation. The workflow execution engine 1506 advantageously blends state machines execution with workflow orchestration. In some implementations, the workflow execution engine 1506 runs a companion workflow and node-level state orchestration on an open-source durable execution platform, thereby delivering durable, reliable execution even across infrastructure failures or downtime. The workflow execution engine 1506 is responsible for initialization that sets up the workflow context; managing execution and transitions between State-style actors; publishing updates are published in real-time for visibility and collaboration; and integration with AI using prompt nodes that call into AI models (e.g., OpenAI, Gemini, etc.). The result is a resilient, auditable workflow that supports both real-time therapy sessions and asynchronous care journeys. The workflow execution engine 1506 is coupled for communication and interaction with the other components 1502, 1504, 1508, 1510, 1512, 1514, 1516, and 1518.

The workflow triggering module 1508 may include software and/or logic to provide functionality for detecting a trigger, determining its condition and initiating a corresponding action in a workflow depending upon the determined condition of the trigger. The workflow triggering module 1508 is particularly advantageous because it allows workflows to trigger other workflows. For example, a given workflow can include event-based triggers (both internal and external); and triggers can dispatch new workflows based on system events or human/AI input; trigger actions outside the system using Webhook integration; direct workflow-to-workflow signals; and call LLM tools. More specifically, event nodes are being designed to trigger external events (e.g., EHR updates, billing systems) and to dispatch downstream workflows. This expands workflows from being a closed systems into orchestrators across enterprise and clinical environments. The workflow triggering module 1508 is coupled for communication and interaction with the other components 1502, 1504, 1506, 1510, 1512, 1514, 1516, and 1518.

The artificial intelligence (AI) module 1510 may include software and/or logic to provide functionality for providing access to artificial intelligence models and tools to any component of a workflow. The workflow builder 902 advantageously fluidly combines AI and human activity into any workflow. Since a given workflow may have prompt nodes and form nodes, the workflow builder 902 seamlessly ensures their cooperation and operation together in the execution of the workflow. The AI module 1310 ensures the seamless integration of AI task in a workflow. The workflow builder 902 may include AI tasks: conversational steps, automatic form completion, contextual reasoning; Human tasks: structured form entry, approvals, or clinician interventions; and/or Transition logic: workflows determine when to advance states based on AI interpretation of patient conversations and inputs. The AI module 1510 enables the inclusion of executable if-then logic at transition points allows us to make explicit why a given AI-to-human or AI-to-next-step transition occurs. This improves explainability and supports regulatory compliance. The AI module 1510 is coupled for communication and interaction with the other components 1502, 1504, 1506, 1508, 1512, 1514, 1516, and 1518.

The data persistence and collaboration module 1512 may include software and/or logic to provide functionality for monitoring the operation of the workflow builder 902 and storing workflow information and form information in the workflow library 1518 and the forms library 1516, respectively. The data persistence and collaboration module 1512 may store any number of interim versions of the workflow during this creation with different states, nodes, transition, variables, or other information that has been added for a given workflow. In some implementations, versions of the workflow may be stored upon request by the user, or automatically by the system periodically, or at defined points within workflow creation and testing. In some implementations, workflows are versioned, stored as JSON, and support Snapshots for history and rollback; Real-time collaborative editing (multiple designers working together) and Tag-based organization and se arch. This makes them living, shareable clinical assets. The data persistence and collaboration module 1512 is coupled for communication and interaction with the other components 1502, 1504, 1506, 1508, 1510, 1514, 1516, and 1518.

The other modules 1514 may include software and/or logic to provide functionality for performing other operations related to workflows, nodes, transitions and data. For example, The other modules 1514 may include plugin systems for external AI tools and models; new node types (e.g., integration nodes for HL7/FHIR, billing nodes, etc.); enterprise-level controls (role-based access, multi-tenant sharing), and expanded explainability tooling, leveraging the if-then execution model to provide transparency around AI behavior. The other modules 1514 are coupled for communication and interaction with the other components 1502, 1504, 1506, 1508, 1510, 1512, 1516, and 1518.

The forms library 1516 may include software and/or logic to provide functionality for storing and retrieving forms from temporary or permanent storage. The forms library 1516 stores a collection of pre-written or partially written forms. In some implementations, the forms library 1516 may be a database stored in any one of the data sources 135 or locally as part of the AI hybrid care management server 120. The forms library 1516 is coupled for communication and interaction with the other components 1502, 1504, 1506, 1508, 1510, 1512, 1514, and 1518.

The workflow library 1518 may include software and/or logic to provide functionality for storing and retrieving workflows from temporary or permanent storage. The workflow library 1518 stores a collection of pre-written or partially written workflows. For example, in an implementation, the workflow library 1518 includes any number of workflow templates which may include one or more prompt nodes, tool calls, forms, if/then conditions as well as accompanying scenarios and/or graders. These are all designed to deliver an interactive conversational AI experience to the patient aligned to the parameters of intended clinical interactions and/or goals. In some implementations, the workflow library 1518 may be a database stored in any one of the data sources 135 or locally as part of the AI hybrid care management server 120. The workflow library 1518 is coupled for communication and interaction with the other components 1502, 1504, 1506, 1508, 1510, 1512, 1514, and 1516.

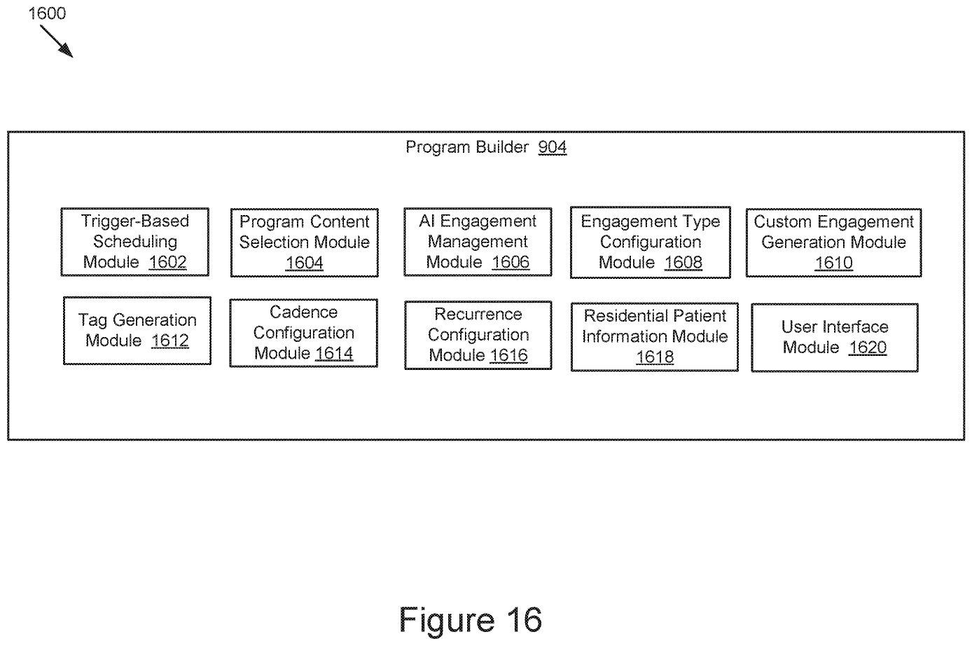

FIG. 16 is a block diagram illustrating an implementation of a program builder in more detail. Referring now to FIG. 16, the program builder 904 according to some implementations will be described. In some implementations, the program builder 904 comprises a trigger-based scheduling module 1602, a program content selection module 1604, an AI engagement management module 1606, an engagement type configuration module 1608, a custom engagement generation module 1610, a tag generation module 1612, a cadence configuration module 1614, a recurrence configuration module 1616, a residential patient information module 1618, and a user interface module 1620. These components of the program builder 904 are coupled for communication, interaction, and cooperation with each other and with the other components of the AI hybrid care management server 120b.

As noted above, different configurations of these components and/or elements of the program builder 904 may be operational on the AI hybrid care management server 120b alone, the clinician device 145a alone, the client device 115a alone, or any combination of different component on either the AI hybrid care management server 120b, the clinician device 145 or the client device 115. In one preferred example implementation, the program builder 904 enables lightweight integration of the AI therapist assistant 150 to a 3^{rd} party EMR 180: In such an implementation, the AI therapist assistant 150(1) gets context from and read/write to the EMR/virtual meeting platform 170, and (2) the AI Therapist 150 engages the patient/therapist in the session. Various implementations of read-write to the EMR for purposes of (1) providing context to the AI Therapist Assistant 150, and/or (2) recording important information to the medical record (Session Monitoring Module 128), and/or (3) triggering a crisis or escalation workflow, and/or (4) triggering a Task to be assigned to one or more persons, roles or groups, and/or (5) scheduling follow-up engagements, and/or (6) triggering a workflow instance of any number of defined workflow templates. Browser plug in which reads the Document Object Model (DOM) or otherwise interprets a 3^{rd} party EMR web interface and reads and writes to it. API integration of the AI Hybrid Care Management Server 120b with a 3^{rd} party EMR Management Module 180. Various implementations of enabling an AI Therapist Assistant, supported on one platform via an AI Hybrid Care Management Service 120b, to engage via voice or text with a therapist and/or patient via a separate Virtual Meeting Platform 160. The AI Hybrid Care Management Service 120b reads the schedule and dial-in information for appointments via the read-write methods to the 3^{rd} party EMR 180 and/or the virtual meeting platform 160 and/or another source of information on (i) the appointment start and end times, (ii) the patient and therapist identification information, and (iii) the information needed to access and authenticate into the appointment (e.g., via dial-in or web VoIP engagement), and then uses this information to have the AI Therapist Assistant 150 join the session as a participant either via web VoIP engagement or via phone dial-in. The therapist enters the web address and/or phone number and/or appointment start time and/or patient identifying information of an appointment to the Therapeutic Chat Interface Application 110b and thereby causes the AI Therapist Assistant to dial in or join the session via the Session Routing Module 122.

The trigger-based scheduling module 1602 may include software and/or logic to provide functionality for enabling trigger-based scheduling for one or more program content blocks and/or other AI engagements as part of building a program. For example, the trigger-based scheduling module 1602 enables a program content block to be executed upon completion of an event, upon a triggering event, or upon detection of a customized trigger. An event may include a scheduled admission into a residential treatment facility, for example. Other events may include completion of an AI hybrid session, the scheduling of an appointment with a therapist, and any number of customizable events. The trigger-based scheduling module 1602 may rely on the workflow builder 902 to enable trigger-based scheduling of program content. The trigger-based scheduling module 1602 is coupled for communication and interaction with the other components 1604, 1606, 1608, 1610, 1612, 1614, 1616, 1618, and 1620.

The program content selection module 1604 may include software and/or logic to provide functionality for enabling program content selection of a program that is being built by a user. For example, program content blocks may include various types of program content, including various mental health exercises, sequences of questions that enable the patient to explore various issues related to their mental health, cognitive behavioral therapy exercises that include various protocols to enable the patient to address cognitive patterns, custom proprietary assessments, and so on. The program content blocks may be configured, in an implementation, through the workflow builder 902 to include the questions and various responses to patient answers. In some implementations, the program content blocks may be created through the workflow builder 902 while selecting program content through the program content selection module 1604. The program content selection module 1104 is coupled for communication and interaction with the other components 1602, 1606, 1608, 1610, 1612, 1614, 1616, 1618, and 1620.

The AI engagement management module 1606 may include software and/or logic to provide functionality for managing the different AI engagement programs that are created, configured, and stored for future use through the program builder 904. For example, a program that is delivered through an AI therapist and/or AI therapist assistant may be an AI engagement that has a status (e.g., active, draft, pending approval, inactive), a date created, a date updated, an identified user that created the program, and tags that are associated with the program, including tags that identify a mental health issue, tags that identify different stages of treatment (e.g., intake, detox, check-in, check-out, etc.) and tags that are associated with the AI therapist assistant, AI therapist, and/or human therapist(s). Through the AI engagement management module 1606, a user may create a new program, view and/or modify existing programs, create engagements within a program, select a schedule for the program, add and remove tags for the program, view and edit a program description and name, and assign a specific program to a patient, for example. The AI engagement management module 1606 is coupled for communication and interaction with the other components 1602, 1604, 1608, 1610, 1612, 1614, 1616, 1618, and 1620.

The engagement type configuration module 1608 may include software and/or logic to provide functionality for configuring engagement types for an AI engagement that is being created. For example, certain engagement types may be standard for interacting with patients in a mental health program, such as a pre-session warm up engagement type, a pre-session readiness checklist engagement type, and a homework assignment engagement type to completed after a session. By configuring an engagement type for the new AI engagement that is being created, a user may quickly re-use existing program content blocks, including exercises and programs that have been previously completed by the patient, for example, or the user may customize a specific engagement that includes a specified ordering of one or more program content blocks. In an implementation, the engagement type configuration module 1608 uses the workflow builder 902 to generate the logic and/or create new program blocks while configuring an engagement, for example. The engagement type configuration module 1608 is coupled for communication and interaction with the other components 1602, 1604, 1606, 1610, 1612, 1614, 1616, 1618, and 1620.

The custom engagement generation module 1610 may include software and/or logic to provide functionality for enabling a user to generate a custom engagement with a patient through the AI therapist and/or AI therapist assistant. For example, a custom engagement may include a new engagement type, such as an engagement that occurs based on the AI therapist detecting that the patient is experiencing a crisis. The custom engagement may be generated and stored with a specific name so that it may be used again. Another custom engagement that may be generated includes a specific checklist required to be completed by a particular residential treatment facility. Because different residential treatment facilities have different onboarding procedures, each with different mental health assessments and/or questionnaires, a custom engagement may be generated to capture a customized procedure for the particular residential treatment facility. In other implementations, custom engagements may be generated based on user input from clinicians that use proprietary assessments, exercises, and questionnaires, for example. In yet further implementations, medication assisted treatment may require a protocol to be completed by the patient that can be administered by the AI therapist through a customized engagement, for example. The custom engagement generation module 1610 is coupled for communication and interaction with the other components 1602, 1604, 1606, 1608, 1612, 1614, 1616, 1618, and 1620.

The tag generation module 1612 may include software and/or logic to provide functionality for enabling tags to be generated in association with programs and/or other AI engagements. Through tag-based organization, users of the program builder 904 may search for and discover different program content blocks that have been created for inclusion in their AI engagements. For example, a clinician may tag a program as being related to “anxiety” where the program includes a customized exercise that takes the patient through a calming and grounding exercise, delivered through the AI therapist assistant. In this example, the clinician creates and customizes the program and generates a tag of “anxiety” to associate the program with the mental health condition of “anxiety.” Thus, another user searching for program content related to “anxiety” may discover the customized engagement and incorporate the program content in their own customized program. The tag generation module 1612 is coupled for communication and interaction with the other components 1602, 1604, 1606, 1608, 1610, 1614, 1616, 1618, and 1620.

The cadence configuration module 1614 may include software and/or logic to provide functionality for enabling configuration of a cadence for engaging a patient with the selected one or more program content blocks. A cadence may include a specified amount of time when the program content is attempted to be delivered to a patient. For example, a user may configure the program to be deployed at a cadence of 15 minutes after the program start date/time. In other implementations, the cadence may be any number of minutes before or after the program start date/time. In an implementation, a number of attempts may be made to engage with the patient, and this number may be configured through the cadence configuration module 1614, for example. The cadence configuration module 1614 is coupled for communication and interaction with the other components 1602, 1604, 1606, 1608, 1610, 1612, 1616, 1618, and 1620.

The recurrence configuration module 1616 may include software and/or logic to provide functionality for enabling users to configure a recurrence of an AI engagement with a patient. For example, a clinician may configure an AI engagement that includes one or more program content blocks to be delivered on a recurring basis, such as every week at a certain date and time. The recurrence configuration module 1616 may rely on the workflow builder 902 to generate the recurrence functionality, in an implementation. In another example, upon onboarding a new client, a clinician user may configure a recurring counseling appointment with the patient based on their availability and may modify one or more appointments that have schedule conflicts through the recurrence configuration module 1616. The recurrence configuration module 1616 is coupled for communication and interaction with the other components 1602, 1604, 1606, 1608, 1610, 1612, 1614, 1618, and 1620.

The residential patient information module 1618 may include software and/or logic to provide functionality for retrieving residential patient information from third party systems connected to the AI hybrid care management server 120b and/or system 100b through network 105. Residential patient information may include an admission date associated with the patient. Other residential patient information may include a list of medications that have been prescribed, a history of appointments with clinicians, medical assessments, peer support counseling events, as well as specific information about the patient, such as location, phone number, date of birth, social security number, insurance carrier, habits and/or substance use disorders (SUDs), mental health issues, and referral reason(s). This information may be gathered by the AI therapist and/or AI therapist assistance, in some implementations. This information may also be retrieved from electronic medical records, in various implementations, from data sources 135 connected to the AI hybrid care management server 120b. The residential patient information module 1618 is coupled for communication and interaction with the other components 1602, 1604, 1606, 1608, 1610, 1612, 1614, 1616, and 1620.

The user interface module 1620 may include software and/or logic to provide functionality for enabling users to create and configure programs through one or more user interfaces. For example, a user may create a program and/or AI engagement through a graphical user interface that enables the user to specify various portions of the program, including specifying the engagement type of the new AI engagement and/or mental health program, creating a custom engagement type, selecting a program that already exists, modifying a program to include additional program content blocks, modifying the status of a program, including new program tags, modifying the default schedule of the program, and setting a cadence of deployment of the program. Additionally, because a program may include multiple AI engagements, each AI engagement in the program may be created and/or configured through the same user interface, in an implementation. The user interface module 1620 may rely on the workflow builder 902 to perform the functionality described herein. The user interface module 1620 is coupled for communication and interaction with the other components 1602, 1604, 1606, 1608, 1610, 1612, 1614, 1616, and 1618.

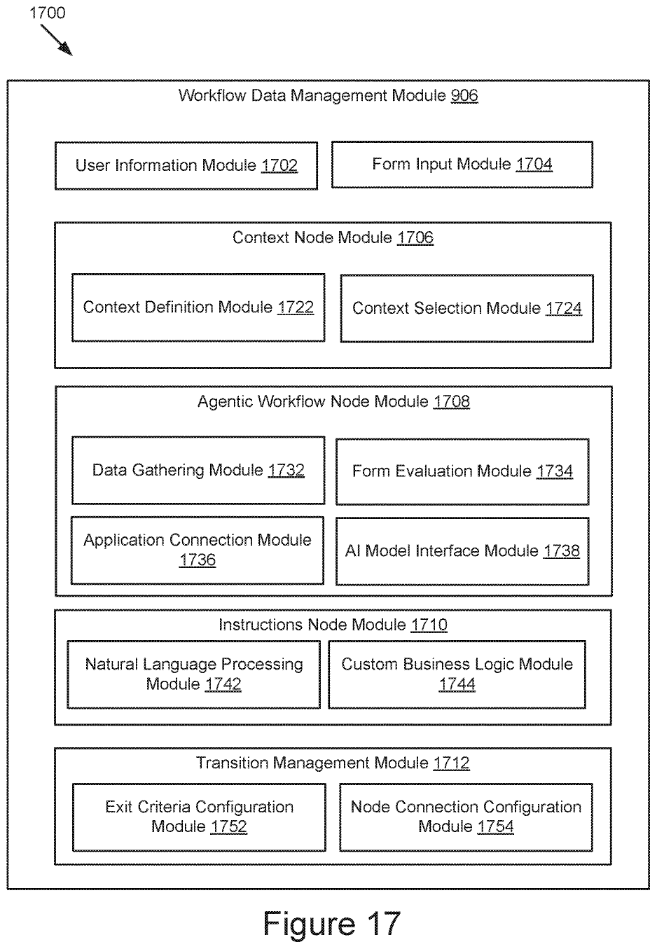

FIG. 17 is a high-level block diagram illustrating an example module for workflow data management according to an embodiment. For example, FIG. 17 illustrates an example workflow data management module 910 that includes a user information module 1702, a form input module 1704, a context node module 1706 that includes a context definition module 1722 and a context selection module 1724, an agentic workflow node module 1708 that includes a data gathering module 1732, an application connection module 1736, a form evaluation module 1734, and an AI model interface module 1738, an instructions node module 1710 that includes a natural language processing module 1742 and a custom business logic module 1744, and a transition management module 1712 that includes an exit criteria configuration module 1752 and a node connection configuration module 1754.

The user information module 1702 may include software and/or logic to provide functionality for retrieving user information for an AI session in mental health treatment. For example, a patient user may have demographic data, electronic medical record data, and historical treatment data stored in a data store accessible by the AI hybrid care management server 120. Other information about the patient user may be retrieved and/or gathered by the user information 1702, such as through third party services on third party servers 140 and/or data sources 135 accessible through the network 105. In other embodiments, the user information module 1702 may retrieve user information stored in a user profile associated with the patient user. In a further embodiment, the user information module 1702 includes other data synthesized and/or summarized from past AI sessions with the patient user.

The form input module 1704 may include software and/or logic to provide functionality for enabling a supervising user to input a form to be executed by the AI companion in the AI session. For example, a supervising user, such as an administrator or therapist, may input a form to be executed in an AI session. The form may include a protocol that must be executed to access medication treatment, in an embodiment. Other forms may include a questionnaire to check for understanding in a recently deployed education module as presented to the patient user. For example, a set of educational sessions on examining core beliefs may be presented through a series of AI sessions. A form may be presented by the form input module 1704 to a patient user after the series of educational sessions to check for understanding and retention of the educational material. Additionally, the form input module 1704 may be used to go through various assessments for intake, behavioral exercises, and other forms that may have already been generated or is standard for filling out by a clinician, for example.

The context node module 1706 may include software and/or logic to provide functionality for enabling a supervising user to add and/or define a context for a particular AI session. For example, a particular AI session may be geared towards psychoeducation. As another example, a response time and format context may be defined with a set of rules in how the AI companion should engage with the patient user, such as keeping responses brief and conversational (e.g., under 120 words) as well as always waiting for a patient user to finish speaking before responding. Different contexts may be defined and stored by the context node module 1706 in a data store accessible by the AI hybrid care management server 120 for future configurations of AI sessions.

The context definition module 1722 may include software and/or logic to provide functionality for defining a context node in a workflow-based configuration of AI sessions in mental health treatment. For example, a context may be defined in plain language to help define the context of an AI session. As mentioned above, some AI sessions are psychoeducational, while others are psychotherapeutic. Any number of context definitions may be created by the context definition module 1722. In one implementation, a workflow may include different phases of intake, admission, and program treatment, for example, that describe different contexts. For example, a context for intake may be an information gathering phase, a pre-session warmup phase, a post-session reflection phase, and so forth. The context definition module 1722 enables a user to define any number of contexts that are useful and relevant for the AI sessions in mental health treatment.

The context selection module 1724 may include software and/or logic to provide functionality for selecting a context node for a particular workflow-based configuration of an AI session. For example, as contexts are defined for AI sessions by the context definition module 1722, a supervising user may select a particular context node for a particular workflow-based configuration of an AI session from previously created context nodes. Alternatively, a new context node may be selected through the context selection module 1724, where the new context node is defined through the context definition module 1722.

The agentic workflow node module 1708 may include software and/or logic to provide functionality for enabling a supervising user to configure inputs and outputs of one or more AI models used to generate a series of questions and/or responses provided by an AI companion in an AI session for mental health treatment. For example, the agentic workflow node module 1708 may enable a supervising user to generate a new “prompt” node that may include one or more context nodes, be connected to user information as gathered and retrieved by the user information module 1702. The agentic workflow node module 1708 may also enable the supervising user to enable one or more forms that have been inputted to be executed in the AI session. For example, a form that includes a series of questions for executing a protocol to prepare a nurse practitioner or medical professional to administer a new medication may be inputted through the form input module 1704 by a supervising user. The agentic workflow node module 1708 may enable a different supervising user to select the same form to configure an AI session related to a patient user potentially deciding to start a new medication. The agentic workflow node module 1708 may further enable a supervising user to provide instructions to the agentic AI in plain language on how to conduct the AI session. The instructions may be received through the graphical user interface in the workflow-based configuration of the AI session and may be executed by the instructions node module 1710, as described below. Further, the agentic workflow node module 1708 may enable a supervising user to select which inputs and outputs are connected to the “prompt” node and establish exit criteria for transitions to additional workflow nodes. The supervising user may select their desired inputs through the graphical user interface by connecting nodes for input, such as a user information node, one or more context nodes, and/or results of applications to the prompt node on the left hand side, as an example. The supervising user may similarly select their desired outputs and/or transitions through the graphical user interface by connecting the prompt node to other subsequent nodes on the right hand side of the graphical user interface, for example. The agentic workflow node module 1708 is configured to connect the input data to one or more AI models based on the instructions and configuration of the workflow nodes to generate the output data desired by the supervising user configuring the prompt node. For example, an AI model may use the user information and context to generate text for the AI companion to “speak” during the AI session, such as generating short responses under 140 words and generate the responses according to a timing defined in the connected context node. As another example, a different AI model may be instantiated by the agentic workflow node module 366 bas 1708ed on an event listening model in which any speech or utterances by the patient user in the AI session that may indicate the patient user is experiencing a crisis, such as suicidal ideation, threats of harm to themself or others, may be configured through the prompt node in the instructions input field. Other AI models and agents may be connected to the prompt node to utilize an event driven data architecture as configured using the workflow based graphical user interface.

The data gathering module 1732 may include software and/or logic to provide functionality for gathering data to configure the AI session in the workflow-based graphical user interface. As part of the agentic workflow node module 1708, the data gathering module 1732 enables a supervising user to configure various nodes to gather data, such as user information, form responses, and applications that execute psychoeducation, for example.

The application connection module 1736 may include software and/or logic to provide functionality for connecting one or more applications to an AI session through the workflow-based graphical user interface. As part of the agentic workflow node module 1708, the application connection module 1736 enables a supervising user to configure which applications may be connected to the AI session. For example, an application that provides a patient user with a game that incorporates a test of knowledge on substance use disorder may be configured to be connected to an AI session in the event of a particular utterance or other criteria defined by the supervising user. Other types of applications may be connected to the AI session through the application connection module 1736, such as through an application programming interface (API) or other means of connection.

The form evaluation module 1734 may include software and/or logic to provide functionality for enabling the evaluation of a form by a supervising user. For example, a form may include a series of questions about a patient user's past behaviors and responses to various events or triggers. The form evaluation module 1734 is configured to evaluate the responses of the patient user and enable the supervising user to determine next steps based on the evaluation. For example, the responses of the patient user may be rated according to a scale from one to five of severity of response to each question of the form. A question may ask, “Do you ever have thought of inflicting harm to your self or others?” and the response from the patient may be evaluated according to the scale based on a data model or AI model, in one implementation. Based on the patient user's responses to the questions in the form, the form evaluation module 1734 may determine, based on the configuration of the agentic workflow node module 1708 by the supervising user, that the patient user be connected to a human therapist immediately and/or dispatching emergency response services. In an embodiment, a threshold criteria on whether to dispatch emergency response services may be defined by a supervising user in the agentic workflow node module 1708. For example, a certain threshold score that may include a number of points based on the response, in addition to a history of responses to similar questions and/or patient history around self-harm, may be used to determine that the threshold criteria are satisfied. The form evaluation module 1734 is configured to determine whether the threshold criteria are satisfied based on the workflow that has been configured in the workflow builder, in an implementation.

The AI model interface module 1738 may include software and/or logic to provide functionality for configuring one or more AI models that are employed in the AI session. For example, the AI model interface module 1738 may enable a supervising user to invoke one or more AI models through a set of instructions written in plain language. In another embodiment, a graphical user interface may be used to enable a supervising user to configure one or more AI models used in the AI session. For example, an AI model may be generated to analyze the responses received from the patient user in an AI session with an AI companion to generate a summary of the responses for review by a human therapist. That AI model may have parameters that can be modified, such as adjusting the level of specificity of responses generated by the AI companion in response to the patient user in replying to a particular comment made by the patient user, for example. Other types of configurations of an AI model may be generated through the AI model interface module 1738, in an implementation.

The instructions node module 1710 may include software and/or logic to provide functionality for enabling a supervising user to provide instructions on how the AI session should be conducted and what data should be used as input and what data should be outputted, in an implementation. The instructions received may be in plain language that is interpreted by the natural language processing module 1742 to determine an intent of the instructions from the supervising user. Other types of instructions may also be inputted, such as invoking and instantiating an application, making modifications to a standard configuration of one or more AI models, and setting an agenda for the AI session with the patient user of topics and activities to cover in the session.

The natural language processing module 1742 may include software and/or logic to provide functionality for processing plain language received in workflow nodes for configuring the AI session. For example, context may be defined in a context node with plain language that is processed by the natural language processing module 1742 to determine a context of the AI session. As another example, instructions may be received through the prompt node with plain language that may be similarly processed. Any number of internal and/or external natural language processing (NLP) applications and/or software services may be used to power the natural language processing module 1742.

The custom business logic module 1744 may include software and/or logic to provide functionality for defining and executing custom business logic in the workflow-based configuration of AI sessions. For example, custom business logic may be described in plain language as a series of instructions inputted in the graphical user interface for the prompt node. The custom business logic module 1744 may be used to interpret these instructions and transform them into programmatic code and/or actions to be taken with data, AI models, and/or internal software applications that power the AI Companion in the AI sessions.

The transition management module 1712 may include software and/or logic to provide functionality for managing the transitions between different “prompt” nodes. In the workflow-based configuration of the AI session, different prompt nodes may be configured to enable the supervising user to instruct the AI Companion to respond to or handle different situations that may arise in the AI session. For example, the transition management module 1712 may direct the AI Companion to execute a different prompt node based on various exit criteria, such as any indication of a crisis based on the responses in the AI session. Additionally, a maximum number of “turns” may be configured that limit the number of responses generated for a particular prompt node, in an implementation.

The exit criteria configuration module 1752 may include software and/or logic to provide functionality for enabling a supervising user to configure exit criteria for a prompt node in a workflow-based configuration of an AI session. For example, exit criteria may be received as instructions in plain language by the supervising user. The exit criteria configuration module 1752 would transform the instructions into programmatic code, in one implementation, to enable the AI session to “exit” or otherwise transition to the next node in the workflow.

The node connection configuration module 1754 may include software and/or logic to provide functionality for configuring how nodes in the workflow-based configuration of AI sessions may be connected to other nodes. As described above, the nodes are represented as blocks on the graphical user interface and connected by the supervising user on the left hand side of the node to configure inputs as well the right hand side of the node to configure outputs.

FIGS. 18A-18E are graphical representations of example user interfaces of a supervising user accessing workflow-based configuration of agentic AI sessions in mental health treatment. For example, FIG. 18A illustrates an example screen 1800 showing an example graphical user interface used in workflow-based configuration of agentic AI sessions in mental health treatment. Different types of nodes are listed in the node library on the left hand side of the screen 1800, including a prompt node for creating AI instructions/prompts, a context node for creating context for the AI, a condition node for creating conditions for the AI, a custom form node for creating custom forms for the AI, and an event transition node that listens for events and transitions to different prompts in the flow. As illustrated in FIG. 18A, a start node begins the workflow on the left side of screen 1800. A response time & format context node is connected to the start node, with a context defined in a text box in the graphical user interface element for the response time & format context node. A user info node appears above the response time & format context node, enabling the supervising user to retrieve and/or gather information about the patient user to provide additional context to the agentic AI, such as the patient user's name and a summary of one or more past sessions. A psychoeducation context node appears below the response time & format context node that enables the supervising user to define the context of the psychoeducation, including a goal of the session: “practice assertive communication to protect recovery and express needs clearly.” The user info node, response time & format context node, and psychoeducation context node are connected to the “Session 6” prompt node as input. The prompt node enables the supervising user to select which AI companion will be used in the AI session as well as instructions for the AI companion. For example, the instructions include greeting the client and explaining in 20 words or less the goal of the AI session. Finally, a supervising user may “drag and drop” a new node, such as a prompt node, or otherwise modify the workflow-based configuration of the AI session by managing the transitions after the “Session 6” prompt node.

FIG. 18B illustrates a canvas 1810 portion of the workflow-based configuration of the AI session depicted in FIG. 18A.

FIG. 18C illustrates an example screen 1830 that includes the response time & format context node 1820 and psychoeducation context node 1822 as described above. The “Session 6” prompt node 1824 is also shown, in which the output of the user info node, response time & format context node 1820, and psychoeducation context node 1822 are shown as an input at a connection point 1826 on the left hand side of the prompt node 1824. Additionally, instructions graphical user interface element 1828 is shown as part of the prompt node 1824, including the instructions in plain language as a numbered list. Transitions 1832 may be added and configured in the prompt node 1824. A sample of the AI companion's voice may be played in response to the supervising user selecting a button 1834 to play the AI companion's voice sample. As shown in the context portion 1836 of the prompt node 1824, the psychoeducation, response time & format, and user info context nodes have been connected. Lastly, the output of the prompt node may be connected at a connection point 438 on the right hand side of the prompt node 1824.

FIG. 18D illustrates an example screen 1840 that includes the workflow-based graphical user interface of FIGS. 18A-18C. Here, the supervising user has entered new transitions for the “Session 6” prompt node and connected each of them to specific prompt nodes through a connection user interface elements 1842 and 1848, respectively. Prompt node 1844 also includes the output of the instructions inputted at the “Session 6” prompt node at connection point 1846. Thus, the transition management from the “Session 6” prompt node is highly configurable through the workflow-based graphical user interface.

FIG. 18E is an example graphical representation of an example user interface of a supervising user accessing workflow-based configuration of agentic AI sessions in mental health treatment. As shown in screen 1850, a simplified workflow is shown with a form 1852 inputted that executes a psychoeducation protocol around “Core beliefs.” Through the form 1852, the supervising user may provide instructions in plain language that instruct the AI to execute the protocol using certain language, for example.

FIGS. 19A-19C are example graphical representations of an example user interface 1900, 1920, 1940 for assigning the AI therapist assistant 150 to schedule and conduct mental health exercises in accordance with the present disclosure.

FIG. 19A shows an example user interface 1900 that can be used to assign tasks for the AI therapist assistant 150 to perform and the status of those tasks. As shown, the user interface 1900 is one of several tabs 1902. The user interface 1900 has a first section 1904 for assigning tasks for the AI therapist assistant 150 to complete. In some implementations, this first section 1904 includes a selectable button 1906 that allows the therapist to add additional tasks for the AI therapist assistant 150 to complete. In response to the selection of the “+Add New Assignment” button 1906 by the clinician 146, the user interfaces 2000, 2020, 2040, and 2060 as will be described below with reference to FIGS. 20A-20D are presented to the clinician 146 to enable them to create new assignments for the AI therapist assistant 150 and associated parameters for performance of those assignments. The user interface 1900 also includes a second section of current assignments that have been assigned by the therapist and information about that task in terms of what type of task it is, the time it was assigned, and whether additional follow-up attempts were made by the AI therapist assistant 150. In this example, the user interface 1900 has three different tasks that have been assigned that are grouped by different categories. In this example, there are three different types of tasks: 1) homework assignments, 2) pre-session warm-ups, and 3) pre-session readiness checklist. Even though only three types of task are illustrated in the section, this section may include any number of different types of tasks and categories as described above with reference to FIG. 10B. A second section 1910 shows the completed tasks. The second section 1910 also shows similar information to the current assignments section 1908 for each task including its time, assignment/creation date, timing, and notes.

FIG. 19B shows an example user interface 1920 that can be used to assign tasks for the AI therapist assistant 150 to perform. In this example, the user interface 1920 has the same header and is one tab of a number of tabs. The user interface 1920 also has the selectable button 1906 for adding new tasks and listing the current assignments of uncompleted tasks. In this implementation, the user interface 1920 does not show the completed tasks or the history of what the AI therapist assistant 150 has performed.

FIG. 19C shows an example user interface 1940 that can be used to show the history of tasks the AI therapist assistant 150 has performed. In this example, the user interface 1940 has the same header and is one tab of a number of tabs. The user interface 1940 does not provide ability to assign new tests and only shows the historical tasks that have been completed. FIGS. 19B and 19C are provided to illustrate how different components of the user interface 1900 of FIG. 19A may be segregated out individually or be used in combination with other user interfaces.

FIGS. 20A-20D show example graphical representations of user interfaces 2000, 2020, 2040, and 2060 for creating new assignments for the AI therapist assistant 150 to perform.

FIG. 20A shows an example graphical representations of user interface 2000 that may be presented to the clinician 146 once they have selected the add assignment button 1906. FIG. 20A is merely one example that includes a window 2002 that allows the clinician 146 to select an assignment type to create. In this example, there are three types of assignments and an associated button 2004, 2006, and 2008 to create each type of assignment. A first button 2004 allows the clinician 146 to create a “homework assignment.” Selection of the first button 2004 causes the user interface to transition from the window 2002 to the user interface 2020 shown in FIG. 20B. A second button 2006 allows the clinician 146 to create a “pre-session warm-up” assignment. Selection of the second button 2006 causes the user interface to transition from the window 2002 to the user interface 2040 shown in FIG. 20C. A third button 2008 allows the clinician 146 to create a “pre-session readiness checklist” assignment. Selection of the third button 2008 causes the user interface to transition from the window 2002 to the user interface 2060 shown in FIG. 20C. As noted above, the user interface of FIG. 20A is merely one example of a user interface 2000 for creating pass by a clinician. Other implementations of the user interface may have other types of assignments, fewer assignment buttons or more assignment buttons.

FIG. 20B shows an example graphical representations of user interface 2020 that may be presented to the clinician 146 to allow them to input or provide additional requirements or parameters or creation of a “homework assignment.” As shown, the user interface 2020 includes a label 2022 identifying that the window is for use in creating a new assignment and that the assignment type is a homework assignment. The user interface 2020 includes a variety of parameter labels and fields for defining the parameters associated with the assignment including: a specific homework assignment to identify the homework assignment, a date and time for the first attempt to contact the user 106, a number of times to attempt to contact the user 106 to complete the assignments, a preferred time of day to contact the user, a notes field, and selectable buttons 2024 to either create the assignment or cancel this creation of the assignment.

FIG. 20C shows an example graphical representations of user interface 2040 that may be presented to the clinician 146 to allow them to input or provide additional requirements or parameters or creation of a “Pre-Session Warm Up” assignment. As shown, the user interface 2040 includes a label 1642 identifying that the window is for use in creating a new assignment and that the assignment type is a “Pre-Session Warm Up” assignment. The user interface 2040 includes a variety of parameter labels and fields for defining the parameters associated with the assignment including: a field for the clinician 146 to select the warm up activity, a field for the amount of time before the appointment at which to begin the warm up activity, a field to select the appointment associated with the warm up activity, a notes field, and selectable buttons 2024 to either create the assignment or cancel this creation of the assignment.

FIG. 20D shows an example graphical representations of user interface 2060 that may be presented to the clinician 146 to allow them to input or provide additional requirements or parameters or creation of a “Pre-Session Readiness Checklist” assignment. As shown, the user interface 2060 includes a label 2062 identifying that the window is for use in creating a new assignment and that the assignment type is a “Pre-Session Readiness Checklist” assignment. The user interface 2060 includes a variety of parameter labels and fields for defining the parameters associated with the assignment including: a field for the clinician 146 to select the readiness checklist items, a field for the amount of time before the appointment at which to begin completing the readiness checklist, a field to select the appointment associated with the readiness checklist, a notes field, and selectable buttons 2024 to either create the assignment or cancel this creation of the assignment.

Referring now to FIG. 21, an example user interface 2100 for creating a workflow using the workflow builder 902 will be described. FIG. 21 shows the example user interface 2100 having a window including a header, a first sidebar, a series of pulldown menus, the second sidebar presenting a node library 2102, and a canvas or workspace 2106. The user interface 2100 is particularly advantageous because it allows a non-technical person to create a workflow using drag-and-drop functionality. Any of the node buttons 2104 in the note library 2102 may be selected and dragged to the canvas 2106 to add a note to the workflow 2130. For example, buttons are provided for the creation of specific nodes including a button 2104a for a prompt node, a button 2104b for a context node, a button 2104c or a condition node, a button 2104d for a form node, a button 2104e for a events node, and a button 2104f for an integration. As shown, the node library 2102 may include any number of additional buttons or other items including variables, sticky notes, MCP servers, etc. FIG. 21 also shows an example workflow 2130 that has been created by the user. The workflow 2130 includes a node 2108 for variable information, a plurality of transitions 2110 connecting nodes, a start node 2112, a plurality of prompt nodes 2114a-2114c, nodes for a system prompt and a session prompt 2116, a node for a referral contact 2118, and a note for a caller type 2120. The workflow 2130 illustrates how the user can combine any number of different types of nodes of any number of different transitions. The canvas 2106 also includes a sticky note proximate the bottom left corner. The nodes are easy to connect with transitions, and thus, easy for a nonprogrammer to create workflows. Moreover, the user interface 2100 also illustrates how the user can take an existing workflow that they are satisfied with and make several modifications to nodes and transitions to perfect or enhance an existing workflow to suit their needs. The user interface 2100 is merely one example of how the workflow builder 902 may be used to present its constituent components or creation and modification by the user.

A system and method for providing patients with access to care for appropriate healthcare services using an intelligent hybrid therapeutic chat interface has been described, specifically coordinating one or more of therapy, medication assisted treatment, and other clinical or administrative engagements with the support of an AI Assistant that collaborates with the clinician, the patient, and/or administrative staff. In the above description, for purposes of explanation, numerous specific details are set forth in order to provide a thorough understanding of the techniques introduced above. It will be apparent, however, to one skilled in the art that the techniques can be practiced without these specific details. In other instances, structures and devices are shown in block diagram form in order to avoid obscuring the description and for ease of understanding. For example, the techniques are described in one implementation above primarily with reference to software and particular hardware. However, the present invention applies to any type of computing system that can receive data and commands, and present information as part of any peripheral devices providing services.

Reference in the specification to “one implementation” or “an implementation” means that a particular feature, structure, or characteristic described in connection with the implementation is included in at least one implementation. The appearances of the phrase “in one implementation” in various places in the specification are not necessarily all referring to the same implementation.

Some portions of the detailed descriptions described above are presented in terms of algorithms and symbolic representations of operations on data bits within a computer memory. These algorithmic descriptions and representations are, in some circumstances, used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing”, “computing”, “calculating”, “determining”, “displaying”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques also relate to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, and magnetic disks, read-only memories (ROMs), random access memories (RAMs), EPROMS, EEPROMs, magnetic or optical cards, flash memories including USB keys with non-volatile memory or any type of media suitable for storing electronic instructions, each coupled to a computer system bus.

The technology described herein can take the form of a hardware implementation, a software implementation, or implementations containing both hardware and software elements.

For instance, the technology may be implemented in software, which includes but is not limited to firmware, resident software, microcode, etc. Furthermore, the technology can take the form of a computer program product accessible from a computer-usable or computer-readable medium providing program code for use by or in connection with a computer or any instruction execution system. For the purposes of this description, a computer-usable or computer readable medium can be any non-transitory storage apparatus that can contain, store, communicate, propagate, or transport the program for use by or in connection with the instruction execution system, apparatus, or device.

A data processing system suitable for storing and/or executing program code may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems, storage devices, remote printers, etc., through intervening private and/or public networks. Wireless (e.g., Wi-Fi™) transceivers, Ethernet adapters, and modems, are just a few examples of network adapters. The private and public networks may have any number of configurations and/or topologies. Data may be transmitted between these devices via the networks using a variety of different communication protocols including, for example, various Internet layer, transport layer, or application layer protocols. For example, data may be transmitted via the networks using transmission control protocol/Internet protocol (TCP/IP), user datagram protocol (UDP), transmission control protocol (TCP), hypertext transfer protocol (HTTP), secure hypertext transfer protocol (HTTPS), dynamic adaptive streaming over HTTP (DASH), real-time streaming protocol (RTSP), real-time transport protocol (RTP) and the real-time transport control protocol (RTCP), voice over Internet protocol (VOIP), file transfer protocol (FTP), WebSocket (WS), wireless access protocol (WAP), various messaging protocols (SMS, MMS, XMS, IMAP, SMTP, POP, WebDAV, etc.), or other known protocols.

Finally, the structure, algorithms, and/or interfaces presented herein are not inherently related to any particular computer or other apparatus. Various general-purpose systems may be used with programs in accordance with the teachings herein, or it may prove convenient to construct more specialized apparatus to perform the required method blocks. The required structure for a variety of these systems will appear from the description above. In addition, the specification is not described with reference to any particular programming language. It will be appreciated that a variety of programming languages may be used to implement the teachings of the specification as described herein.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects are not mandatory or significant, and the mechanisms that implement the specification or its features may have different names, divisions and/or formats.

Furthermore, the modules, routines, features, attributes, methodologies, engines, and other aspects of the disclosure can be implemented as software, hardware, firmware, or any combination of the foregoing. Also, wherever an element, an example of which is a module, of the specification is implemented as software, the element can be implemented as a standalone program, as part of a larger program, as a plurality of separate programs, as a statically or dynamically linked library, as a kernel loadable module, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A computer-implemented method, comprising:
receiving input to begin a hybrid therapeutic session, wherein a therapeutic chat interface application comprises an artificial intelligence agent configured to manage the hybrid therapeutic session;
executing, by the artificial intelligence agent, a plurality of workflows applicable to the user based on processing the received user input using a machine learning model comprising a natural language understanding model, the plurality of workflows including at least one evidence-based exercise associated with a plurality of health conditions including at least one mental health condition;
generating one or more responses based on the at least one evidence-based exercise associated with the plurality of health conditions, the one or more responses generated using a generative artificial intelligence model by the artificial intelligence agent;
presenting the one or more responses to a user through the therapeutic chat interface application through the artificial intelligence agent;
receiving one or more first user interactions to the one or more responses through the therapeutic chat interface application;
sending a first notification to a first clinician user to join the hybrid therapeutic session, the first clinician user tasked with addressing the at least one mental health condition;
providing a first transition within the therapeutic chat interface application by the artificial intelligence agent, the transition including a first plurality of generated statements summarizing the one or more first user interactions to introduce the first clinician user to the user within the hybrid therapeutic session; and
generating a summary of the hybrid therapeutic session associated with the user of the therapeutic chat interface application, the summary of the hybrid therapeutic session generated by the artificial intelligence agent.

2. The method of claim 1, further comprising:
providing the summary of the hybrid therapeutic session associated with the user to a second clinician user through the therapeutic chat interface application; and
enabling the second clinician user to generate a prescription associated with the medication assisted treatment through the therapeutic chat interface application after reviewing the summary of the hybrid therapeutic session with the user.

3. The method of claim 1, further comprising:
responsive to the second clinician user asynchronously requesting additional information from the user, generating a series of prompts based on the additional information requested from the user of the therapeutic chat interface application, the series of prompts generated using the generative artificial intelligence model;
providing the series of prompts to the user through the therapeutic chat interface application; and
sending one or more user responses to the series of prompts to the second clinician user through the therapeutic chat interface application.

4. The method of claim 1, further comprising:
responsive to the second clinician user requesting to schedule an appointment with the user through the therapeutic chat interface application, sending an invitation to schedule the appointment from the second clinician user to the user in the hybrid therapeutic session through the therapeutic chat interface application.

5. The method of claim 1, further comprising:
generating a summary of user activity associated with the user of the therapeutic chat interface application, the summary of user activity generated using a generative artificial intelligence model;
providing the summary of user activity associated with the user to a third clinician user through the therapeutic chat interface application; and
providing a third transition within the therapeutic chat interface application by the artificial intelligence agent, the transition including a third plurality of generated statements to introduce the third clinician user to the user within the hybrid therapeutic session through the therapeutic chat interface application.

6. The method of claim 5, further comprising:
based on clinician user activity, the clinician user activity including clinician user interactions received from at least one of the first clinician user, the second clinician user, and the third clinician user, delivering a protocol associated with the medication assisted treatment to the user;
based on the delivered protocol associated with the medication assisted treatment, generating a medical billing record associated with the user; and
sending the medical billing record to the second clinician user associated with the medication assisted treatment.

7. The method of claim 1, further comprising:
generating an audit trail associated with licensed treatment provided to the user, the audit trail comprising a log of user interactions in the hybrid therapeutic session received through the therapeutic chat interface application;
determining one or more prompts associated with the licensed treatment;
presenting the one or more prompts associated with the licensed treatment through the therapeutic chat interface application to the first clinician user and the second clinician user;
receiving from at least one of the first clinician user and the second clinician user a plurality of user responses to the one or more prompts associated with the licensed treatment through the therapeutic chat interface application; and
storing the plurality of user responses from the at least one of the first clinician user and the second clinician user in a data store.

8. The method of claim 1, further comprising:
determining an indication to coordinate review of user activity associated with the user based on the received one or more user interactions to the one or more responses through the therapeutic chat interface application;
generating a plurality of prompts associated with the review of user activity;
presenting the plurality of prompts associated with the review of user activity through the therapeutic chat interface application to a plurality of clinician users;
receiving a plurality of clinician user responses to the plurality of prompts associated the review of user activity through the therapeutic chat interface application; and
storing the plurality of clinician user responses in a data store.

9. The method of claim 1, further comprising:
receiving one or more second user interactions to one or more transcribed statements made by the first clinician user in the hybrid therapeutic session through the therapeutic chat interface application; and
determining a prompt to suggest a statement to be made by the first clinician user through the therapeutic chat interface application, the prompt generated by a triggered workflow of the plurality of workflows, the triggered workflow addressing the at least one mental health condition.

10. A computer-implemented method, comprising:
providing a workflow builder interface application to configure an artificial intelligence therapist assistant that orchestrates a hybrid therapeutic session through a therapeutic chat interface application, the workflow builder interface application comprising a user interface and a plurality of nodes associated with a plurality of functions in the therapeutic chat interface application;
receiving user input from an administrative user through the workflow builder interface application to configure the artificial intelligence therapist assistant based on one or more nodes of the plurality of nodes placed on the user interface to form a series of workflows, the artificial intelligence therapist assistant configured to manage the hybrid therapeutic session based on the series of workflows generated by the user input received through the workflow builder interface application;
retrieving, by the artificial intelligence therapist assistant, an evidence-based protocol from a data store applicable to the hybrid therapeutic session based on processing the received user input using a machine learning model comprising a natural language understanding model, the evidence-based protocol including a series of exercises and engagements having a clinical therapeutic effect on a patient user;
generating one or more responses associated with the evidence-based protocol, the one or more responses generated using a generative artificial intelligence model by the artificial intelligence therapist assistant;
presenting the one or more responses to the patient user through the therapeutic chat interface application through the artificial intelligence therapist assistant;
receiving one or more user interactions to the one or more responses through the therapeutic chat interface application;
sending a notification to a first clinician user to join the hybrid therapeutic session; and
providing a transition within the therapeutic chat interface application by the artificial intelligence therapist assistant, the transition including a plurality of generated statements to introduce the first clinician user to the patient user within the hybrid therapeutic session.

11. The computer-implemented method of claim 10, further comprising:
receiving one or more second user interactions to one or more transcribed statements made by the first clinician user in the hybrid therapeutic session through the therapeutic chat interface application; and
specifying a prompt to suggest a statement to be made by the first clinician user through the therapeutic chat interface application, the prompt configured by a triggered workflow of the series of workflows, the triggered workflow associated with the clinical therapeutic effect on the patient user.

12. The computer-implemented method of claim 10, further comprising:
determining an indication to generate a mental health program for the patient user;
selecting one or more program content blocks based on the indication to generate the mental health program for the patient user;
configuring the one or more program content blocks related to the patient user based on user input received through a program building interface provided through the workflow builder interface application;
generating a program timeline to deploy the mental health program comprising the one or more program content blocks in a user-configurable sequence to the patient user within the therapeutic chat interface application by the artificial intelligence therapist assistant;
retrieving an appointment availability for the first clinician user to engage with the patient user following completion of the one or more program content blocks;
based on the appointment availability, generating a schedule associated with the program timeline to deploy the mental health program to the patient user; and
providing the schedule associated with the program timeline for display on the program building interface through the workflow builder interface application.

13. The computer-implemented method of claim 10, further comprising:
determining an indication to support clinical engagement with the patient user based on the received one or more user interactions; and
generating one or more activities to support clinical engagement with the patient user within the therapeutic chat interface application by the artificial intelligence therapist assistant.

14. The computer-implemented method of claim 10, further comprising:
determining an indication to perform one or more pre-session activities with the patient user based on the received one or more user interactions; and
generating the one or more pre-session activities to engage with the patient user within the therapeutic chat interface application by the artificial intelligence therapist assistant.

15. The computer-implemented method of claim 10, further comprising:
determining an indication to perform one or more in-session activities with the patient user based on the received one or more user interactions; and
generating the one or more in-session activities to engage with the patient user within the therapeutic chat interface application by the artificial intelligence therapist assistant.

16. The computer-implemented method of claim 10, further comprising:
determining an indication to perform one or more post-session activities with the patient user based on the received one or more user interactions; and
generating the one or more post-session activities to engage with the patient user within the therapeutic chat interface application by the artificial intelligence therapist assistant.

17. The computer-implemented method of claim 10, further comprising:
determining an indication to perform one or more additional exercises with the patient user based on the received one or more user interactions; and
generating the one or more additional exercises based on the retrieved evidence-based protocol to engage with the patient user within the therapeutic chat interface application by the artificial intelligence therapist assistant.

18. A computer-implemented method, comprising:
providing a workflow builder interface application to configure an artificial intelligence therapist assistant that orchestrates a hybrid therapeutic session through a therapeutic chat interface application, the workflow builder interface application comprising a graphical user interface and a plurality of nodes associated with a plurality of functions in the therapeutic chat interface application;
receiving user input from an administrative user through the workflow builder interface application to configure the artificial intelligence therapist assistant that orchestrates the hybrid therapeutic session through the therapeutic chat interface application, the artificial intelligence therapist assistant based on one or more nodes of the plurality of nodes placed on the graphical user interface to form a series of workflows, the artificial intelligence therapist assistant configured to manage the hybrid therapeutic session based on the series of workflows generated by the user input received through the workflow builder interface application;
processing the one or more nodes of the plurality of nodes placed on the graphical user interface to form the series of workflows, the one or more nodes interpreted by an artificial intelligence agent to retrieve a workflow based on a published protocol for the artificial intelligence therapist assistant to execute within the hybrid therapeutic session;
specifying a series of prompts based on the processed one or more nodes interpreted by the artificial intelligence agent, the series of prompts customized to a patient user based on a history of transcribed conversations, profile information about the patient user, and the retrieved workflow based on the published protocol;
storing, by the artificial intelligence therapist assistant, the series of prompts as a new custom protocol at a data store, the new custom protocol including the published protocol customized to the patient user;
generating one or more responses associated with the new custom protocol, the one or more responses generated using a generative artificial intelligence model by the artificial intelligence therapist assistant;
presenting the one or more responses to the patient user through the therapeutic chat interface application through the artificial intelligence therapist assistant;
receiving one or more user interactions to the one or more responses through the therapeutic chat interface application;
sending a notification to a first clinician user to join the hybrid therapeutic session; and
providing a transition within the therapeutic chat interface application by the artificial intelligence therapist assistant, the transition including a plurality of generated statements to introduce the first clinician user to the patient user within the hybrid therapeutic session.

19. The computer-implemented method of claim 18, further comprising:
receiving one or more second user interactions to one or more transcribed statements made by the first clinician user in the hybrid therapeutic session through the therapeutic chat interface application;
determining a suggested statement for the first clinician user through the therapeutic chat interface application, the suggested generated by a triggered workflow of the series of workflows based on the one or more second user interactions received, the triggered workflow associated with the retrieved workflow based on the new custom protocol; and
providing the suggested statement to the first clinician user through the therapeutic chat interface application.

20. The computer-implemented method of claim 18, further comprising:
determining an indication to perform one or more additional engagements with the patient user based on the received one or more user interactions; and
generating the one or more additional engagements by another artificial intelligence agent based on the generative artificial intelligence model associated with the new custom protocol to engage with the patient user within the therapeutic chat interface application by the artificial intelligence therapist assistant.
